SEQUENCE LISTING (3 sequences)

SEQ ID NO: 1 - protein, 393 aa, Artificial Sequence
  Synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSENLYFQGMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 2 - protein, 104 aa, Artificial Sequence
  Synthetic construct
MDYKDDDDKYVKLISSDGHEFIVKREHALTSGTIKAMLSGPGQFAENETNEVNFREIPSHVLSKVCMYFTYKVRYTNSSTEIPEFPIAPEIALELLMAANFLDC

SEQ ID NO: 3 - protein, 146 aa, Artificial Sequence
  Synthetic construct
MWSHPQFEKGGGSGGGSGGGSWSHPQFEKDVFLMIRRHKTTIFTDAKESSTVFELKRIVEGILKRPPDEQRLYKDDQLLDDGKTLGECGFTSQTARPQAPATVGLAFRADDTFEALCIEPFSSPPELPDVMKPQDSGSSANEQAVQ

(19) United States
(12) Patent Application Publication
(10) Pub. No.: US 2024/0002458 A1
(43) Pub. Date: Jan. 4, 2024

(54) NOVEL VHL SMALL MOLECULE PROBES

(71) Applicant: St. Jude Children's Research Hospital, Memphis, TN (US)

(72) Inventors: Taosheng Chen, Germantown, TN (US); Wenwei Lin, Memphis, TN (US); Yongtao Li, Memphis, TN (US)

(21) Appl. No.: 18/035,502

(22) PCT Filed: Nov. 8, 2021
(86) PCT No.: PCT/US2021/058486

§ 371 Date: May 4, 2023

Related U.S. Application Data
(60) Provisional application No. 63/111,407, filed on Nov. 9, 2020.

(51) Int. Cl. C07K 14/47; G01N 33/58; G01N 33/542

(52) U.S. Cl. CPC C07K 14/4705; G01N 33/582; G01N 33/542; C07K 2319/60; G01N 2500/00

(57) ABSTRACT

The present disclosure relates to compositions and methods for use in modulating von Hippel-Lindau protein (pVHL) and in identifying pVHL ligands, which can be useful in, for example, treating anti-chronic anemia and anti-chronic ischemia, and also as proteolysis targeting chimeras (PROTACS) to degrade proteins for various therapeutic applications. This abstract is intended as a scanning tool for purposes of searching in the particular art and is not intended to be limiting of the present invention.

### CROSS-REFERENCE TO RELATED APPLICATIONS

This application claims the benefit of U.S. Application No. 63/111,407, filed on Nov. 9, 2020, the contents of which are hereby incorporated by reference in their entirety.

### REFERENCE TO SEQUENCE LISTING

The Sequence Listing submitted Nov. 8, 2021 as a text file named “19116_0047P1_ST25.txt,” created on Nov. 4, 2021, and having a size of 8,192 bytes is hereby incorporated by reference pursuant to 37 C.F.R. § 1.52(e)(5).

### BACKGROUND

The von Hippel-Lindau protein (pVHL) associates with transcription factors elongin C and elongin B to form the VCB protein complex (Kibel, et al. (1995). Binding of the von Hippel-Lindau tumor suppressor protein to Elongin B and C. Science 269, 1444-1446; Stebbins, et al. (1999) Structure of the VHL-ElonginC-ElonginB complex: implications for VHL tumor suppressor function. Science 284, 455-461), which is critical for the function of pVHL, such as degrading its substrates hypoxia-inducible factors (HIFs) (Maxwell, et al. (1999) The tumour suppressor protein VHL targets hypoxia-inducible factors for oxygen-dependent proteolysis. Nature 399, 271-275). Ligands of VHL have been used to modulate VHL-HIF1α interaction (Buckley, et al. (2012) Targeting the von Hippel-Lindau E3 ubiquitin ligase using small molecules to disrupt the VHL/HIF-1α interaction. J Am Chem Soc 134, 4465-4468; Buckley, et al. (2012) Small-molecule inhibitors of the interaction between the E3 ligase VHL and HIF1α. Angew Chem Int Ed Engl 51, 11463-11467; Van Molle, et al. (2012) Dissecting fragment-based lead discovery at the von Hippel-Lindau protein:hypoxia inducible factor 1α protein-protein interface. Chem Biol 19, 1300-1312; Galdeano, C., et al. (2014) Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities. J Med Chem 57, 8657-8663; Frost, J, et al. (2016) Potent and selective chemical probe of hypoxic signalling downstream of HIF-α hydroxylation via VHL inhibition. Nat Commun 7, 13312) and regulate hypoxic signaling with potential applications as anti-chronic anemia or anti-ischemia. These VHL ligands, represented by VH032 (1) and VH298 (2), are hydroxyproline (Hyp) derivatives, and Hyp564 of HIF-1α is known to be critical for its interaction with VHL (Min, J, et al. (2002) Structure of an HIF-1alpha-pVHL complex: hydroxyproline recognition in signaling. Science 296, 1886-1889; Hon, et al. (2002) Structural basis for the recognition of hydroxyproline in HIF-1 alpha by pVHL. Nature 417, 975-978). Recently, VHL ligands have also been widely used to generate bivalent molecules as proteolysis targeting chimeras (PROTACs) to degrade proteins for potential therapeutic applications (Wang, Y., et al. (2020) Degradation of proteins by PROTACs and other strategies. Acta Pharm Sin B 10, 207-238). MZ1 (3) is one of such PROTACs (Gadd, et al. (2017) Structural basis of PROTAC cooperative recognition for selective protein degradation. Nat Chem Biol 13, 514-521), which joined a Bromodomain and Extra-Terminal Domain (BET) inhibitor (+)-JQ1 (4) (Filippakopoulos, et al. (2010) Selective inhibition of BET bromodomains. Nature 468, 1067-1073) with the VHL ligand VH032 (1) by a poly(ethylene glycol) (PEG) linker.

To develop ligands for VHL, sensitive and selective assays that measure the binding affinity of ligand to pVHL are critical. Several assays have been reported, including direct binding assays of isothermal titration calorimetry (ITC) (Buckley, et al. (2012) Targeting the von Hippel-Lindau E3 ubiquitin ligase using small molecules to disrupt the VHL/HIF-1α interaction. J Am Chem Soc 134, 4465-4468; Van Molle, I, et al. (2012) Dissecting fragment-based lead discovery at the von Hippel-Lindau protein:hypoxia inducible factor 1α protein-protein interface. Chem Biol 19, 1300-1312; Galdeano, et al. (2014) Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities. J Med Chem 57, 8657-8663; Frost, et al. (2016) Potent and selective chemical probe of hypoxic signalling downstream of HIF-α hydroxylation via VHL inhibition. Nat Commun 7, 13312; Gadd, et al. (2017) Structural basis of PROTAC cooperative recognition for selective protein degradation. Nat Chem Biol 13, 514-521; Soares, et al. (2018) Group-Based Optimization of Potent and Cell-Active Inhibitors of the von Hippel-Lindau (VHL) E3 Ubiquitin Ligase: Structure-Activity Relationships Leading to the Chemical Probe (2S,4R)-1-((S)-2-(1-Cyanocyclopropanecarboxamido)-3,3-dimethylbutanoyl)-4-hydroxy-N-(4-(4-methylthiazol-5-yl)benzyl)pyrrolidine-2-carboxamide (VH298). J Med Chem 61, 599-618; Testa, et al. (2018) 3-Fluoro-4-hydroxyprolines: Synthesis, Conformational Analysis, and Stereoselective Recognition by the VHL E3 Ubiquitin Ligase for Targeted Protein Degradation. J Am Chem Soc 140, 9299-9313), and surface plasmon resonance (SPR) (Frost, J, et al. (2016) Potent and selective chemical probe of hypoxic signalling downstream of HIF-α hydroxylation via VHL inhibition. Nat Commun 7, 13312; Soares, et al. (2018) Group-Based Optimization of Potent and Cell-Active Inhibitors of the von Hippel-Lindau (VHL) E3 Ubiquitin Ligase: Structure-Activity Relationships Leading to the Chemical Probe (2S,4R)-1-4S)-2-(1-Cyanocyclopropanecarboxamido)-3,3-dimethylbutanoyl)-4-hydroxy-N-(4-(4-methylthiazol-5-yl)benzyl)pyrrolidine-2-carboxamide (VH298). J Med Chem 61, 599-618), and competitive fluorescence polarization (FP) assays which use fluorescently labeled HIF-1α peptides (FAM-DEALAHyp-YIPD, 10-mer, MW: 1477.48 Da; and FAM-DEALAHyp-YIPMDDDFQLRSF, 19-mer, M+H: 2617.167 Da) as the fluorescent binding partners (Buckley, et al. (2012) Targeting the von Hippel-Lindau E3 ubiquitin ligase using small molecules to disrupt the VHL/HIF-1α interaction. J Am Chem Soc 134, 4465-4468; Buckley, et al. (2012) Small-molecule inhibitors of the interaction between the E3 ligase VHL and HIF1α. Angew Chem Int Ed Engl 51, 11463-11467; Van Molle, et al. (2012) Dissecting fragment-based lead discovery at the von Hippel-Lindau protein:hypoxia inducible factor 1α protein-protein interface. Chem Biol 19, 1300-1312; Frost, et al. (2016) Potent and selective chemical probe of hypoxic signalling downstream of HIF-α hydroxylation via VHL inhibition. Nat Commun 7, 13312; Soares, et al. (2018) Group-Based Optimization of Potent and Cell-Active Inhibitors of the von Hippel-Lindau (VHL) E3 Ubiquitin Ligase: Structure-Activity Relationships Leading to the Chemical Probe (2S,4R)-1-4S)-2-(1-Cyanocyclopropanecarboxamido)-3,3-dimethylbutanoyl)-4-hydroxy-N-(4-(4-methylthiazol-5-yl)benzyl)pyrrolidine-2-carboxamide (VH298). J Med Chem 61, 599-618; Crew, et al. (2018) Identification and Characterization of Von Hippel-Lindau-Recruiting Proteolysis Targeting Chimeras (PROTACs) of TANK-Binding Kinase 1. J Med Chem 61, 583-598; Zoppi, V, et al. (2019) Iterative Design and Optimization of Initially Inactive Proteolysis Targeting Chimeras (PROTACs) Identify VZ185 as a Potent, Fast, and Selective von Hippel-Lindau (VHL) Based Dual Degrader Probe of BRD9 and BRD7. J Med Chem 62, 699-726). In all the reported ITC, SPR, and FP assays, the VCB complex was the protein of choice for the optimal interaction. ITC assays typically use high VCB protein concentrations (such as 100 μM) (Buckley, et al. (2012) Targeting the von Hippel-Lindau E3 ubiquitin ligase using small molecules to disrupt the VHL/HIF-1α interaction. J Am Chem Soc 134, 4465-4468), whereas FP assays can utilize VBC protein at concentration as high as 1 μM (Crew, et al. (2018) Identification and Characterization of Von Hippel-Lindau-Recruiting Proteolysis Targeting Chimeras (PROTACs) of TANK-Binding Kinase 1. J Med Chem 61, 583-598) or as low as 15 nM (Frost, et al. (2016) Potent and selective chemical probe of hypoxic signalling downstream of HIF-α hydroxylation via VHL inhibition. Nat Commun 7, 13312). On the other hand, SPR assay requires the immobilization of protein which introduces variations and is not suitable for testing a large number of ligands (Frost, et al. (2016) Potent and selective chemical probe of hypoxic signalling downstream of HIF-α hydroxylation via VHL inhibition. Nat Commun 7, 13312; Soares, et al. (2018) Group-Based Optimization of Potent and Cell-Active Inhibitors of the von Hippel-Lindau (VHL) E3 Ubiquitin Ligase: Structure-Activity Relationships Leading to the Chemical Probe (2S,4R)-1-4S)-2-(1-Cyanocyclopropanecarboxamido)-3,3-dimethylbutanoyl)-4-hydroxy-N-(4-(4-methylthiazol-5-yl)benzyl)pyrrolidine-2-carboxamide (VH298). J Med Chem 61, 599-618).

FP assay can be high throughput, but it has drawbacks. It typically requires higher concentration of target protein than the labeled probe (Buckley, et al. (2012) Targeting the von Hippel-Lindau E3 ubiquitin ligase using small molecules to disrupt the VHL/HIF-1α interaction. J Am Chem Soc 134, 4465-4468; Buckley, et al. (2012) Small-molecule inhibitors of the interaction between the E3 ligase VHL and HIF1α. Angew Chem Int Ed Engl 51, 11463-11467; Van Molle, et al. (2012) Dissecting fragment-based lead discovery at the von Hippel-Lindau protein:hypoxia inducible factor 1α protein-protein interface. Chem Biol 19, 1300-1312; Frost, et al. (2016) Potent and selective chemical probe of hypoxic signalling downstream of HIF-α hydroxylation via VHL inhibition. Nat Commun 7, 13312; Soares, et al. (2018) Group-Based Optimization of Potent and Cell-Active Inhibitors of the von Hippel-Lindau (VHL) E3 Ubiquitin Ligase: Structure-Activity Relationships Leading to the Chemical Probe (2S,4R)-1-4S)-2-(1-Cyanocyclopropanecarboxamido)-3,3-dimethylbutanoyl)-4-hydroxy-N-(4-(4-methylthiazol-5-yl)benzyl)pyrrolidine-2-carboxamide (VH298). J Med Chem 61, 599-618; Crew, et al. (2018) Identification and Characterization of Von Hippel-Lindau-Recruiting Proteolysis Targeting Chimeras (PROTACs) of TANK-Binding Kinase 1. J Med Chem 61, 583-598; Zoppi, et al. (2019) Iterative Design and Optimization of Initially Inactive Proteolysis Targeting Chimeras (PROTACs) Identify VZ185 as a Potent, Fast, and Selective von Hippel-Lindau (VHL) Based Dual Degrader Probe of BRD9 and BRD7. J Med Chem 62, 699-726), which may increase the demand of protein when a large number of ligands need to be tested (Lea, W. A., and Simeonov, A. (2011) Fluorescence polarization assays in small molecule screening. Expert Opin Drug Discov 6, 17-32; Parker, et al. (2000) Development of high throughput screening assays using fluorescence polarization: nuclear receptor-ligand-binding and kinase/phosphatase assays. J Biomol Screen 5, 77-88). Furthermore, FP assays are typically more susceptible to assay interference than TR-FRET assays are (Du, et al. (2011) A dual-readout F² assay that combines fluorescence resonance energy transfer and fluorescence polarization for monitoring bimolecular interactions. Assay Drug Dev Technol 9, 382-393), causing a higher rate of detecting false positives and false negatives (Moerke, N. J. (2009) Fluorescence Polarization (FP) Assays for Monitoring Peptide-Protein or Nucleic Acid-Protein Binding. Curr Protoc Chem Biol 1, 1-15). In contrast, TR-FRET assays usually employ a lot less proteins than the labeled probes and typically utilize low nanomolar proteins (Lin, W., and Chen, T. (2018) Using TR-FRET to Investigate Protein-Protein Interactions: A Case Study of PXR-Coregulator Interaction. Adv Protein Chem Struct Biol 110, 31-63; Lin, et al. (2014) Development of BODIPY FL vindoline as a novel and high-affinity pregnane X receptor fluorescent probe. Bioconjug Chem 25, 1664-1677; Lin, W., and Chen, T. (2013) A vinblastine fluorescent probe for pregnane X receptor in a time-resolved fluorescence resonance energy transfer assay. Anal Biochem 443, 252-260). Moreover, TR-FRET assays have additional advantages of low assay interference due to its time-delayed measurement (Thorne, N., Auld, D. S., and Inglese, J. (2010) Apparent activity in high-throughput screening: origins of compound-dependent assay interference. Curr Opin Chem Biol 14, 315-324) and less well-to-well variation due to its ratiometric detection nature (Glickman, et al. (2002) A comparison of ALPHAScreen, TR-FRET, and TRF as assay methods for FXR nuclear receptors. J Biomol Screen 7, 3-10).

Despite the significance of VHL ligands, sensitive and selective assays to identify such ligands has remained elusive. Thus, there remains a need for methods and probes to identify, develop, and evaluate VHL ligands. These needs and others are met by the instant invention.

### SUMMARY

In accordance with the purpose(s) of the invention, as embodied and broadly described herein, the invention, in one aspect, relates to compositions and methods for use in modulating von Hippel-Lindau protein (pVHL) and in identifying pVHL ligands, which can be useful in, for example, treating anti-chronic anemia and anti-chronic ischemia, and also as proteolysis targeting chimeras (PROTACS) to degrade proteins for various therapeutic applications.

Thus, disclosed are compounds having a structure represented by a formula:

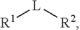

wherein L is a linker; wherein R¹ is a residue of a fluorophore, a residue of biotin, or a residue of a biotin derivative; and wherein R² is a residue of a von Hippel-Lindau protein (pVHL) ligand.

Also disclosed are methods of modulating von Hippel-Lindau protein (pVHL) in a sample, the method comprising contacting the sample with an effective amount of a disclosed compound, thereby modulating VHL protein in the sample.

Also disclosed are methods of identifying a von Hippel-Lindau protein (pVHL) ligand in a library, the method comprising: (a) providing a library that contains a plurality of ligands; (b) combining a disclosed compound and a sample having pVHL, thereby forming a mixture; (c) exposing each ligand to the mixture; and (d) detecting a fluorescence emission of the mixture after exposure to each ligand, wherein a decrease in fluorescence emission indicates that the ligand is a pVHL ligand, and wherein a lack of decrease in fluorescence emission indicates that the ligand is a non-pVHL ligand.

Also disclosed are kits comprising a disclosed compound, and one or more of: (a) a sample that contains von Hippel-Lindau protein (pVHL); (b) a library that contains a plurality of ligands; (c) instructions for modulating pVHL; (d) instructions for identifying a pVHL ligand and/or a non-pVHL ligand; and (e) instructions for performing a fluorescence-based assay.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows representative structures of a panel of VHL ligands.

FIG. 2 shows representative structures of a panel of non-VHL ligands.

FIG. 3 shows a representative schematic illustrating the design of BODIPY FL VH032 (5).

FIG. 4A-C show representative data illustrating the TR-FRET binding affinity investigation of BODIPY FL VH032 (5, 1- to 2-dilutions, an optimized concentration range of 0.06 nM to 500 nM) to GST-VCB.

FIG. 5A-C show representative data illustrating the evaluation of signal stability of the BODIPY FL VH032 (5)-based pVHL TR-FRET assay.

FIG. 6A and FIG. 6B show representative dose response curves of a panel of pVHL ligands and non-pVHL ligands in the presence of BODIPY FL VH032 (5, 4 nM), 2 nM GST-VCB, and 2 nM Tb-anti-GST at a 90-min incubation time; RTUs of ligands at their individual concentrations were normalized to that of VH298 (2, 30 μM, positive control, 100% inhibition) and DMSO (negative control, 0% inhibition) and fitted into a Sigmoidal equation with GraphPad PRISM to derive IC₅₀ values, if applicable; the Kᵢ values were calculated with the Cheng-Prusoff equation (Chan, L. C., and Cox, B. G. (2007) Kinetics of Amide Formation through Carbodiimide/N-Hydroxybenzotriazole (HOBt) Couplings. The Journal of Organic Chemistry 72, 8863-8869).

FIG. 7A and FIG. 7B show representative data illustrating BODIPY FL VH032 (5) concentration optimization in a pVHL FP assay with GST-VCB (1-to-2 dilutions, an optimized concentration range of 0.03 nM to 1000 nM) and a 90-min incubation time.

FIG. 8 shows representative data illustrating the determination of the binding affinity of BODIPY FL VH032 (5, 10 nM) to GST-VCB in an FP assay, using GST-VCB (1-to-2 dilutions, an optimal concentration range of 0.03 nM to 1000 nM)+DMSO (total interactions), GST-VCB (1-to-2 dilutions, an optimal concentration range of 0.03 nM to 1000 nM)+VH298 (2, 30 μM) (GST-VCB-mediated non-specific interaction), or DMSO+without GST-VCB (background interactions) at a 90-min incubation time.

FIG. 9A-D show representative activities of controls and selected pVHL or non-pVHL ligands in the BODIPY FL VH032 (5)-mediated pVHL FP assay with 10 nM BODIPY FL VH032 (5) and 100 nM GST-VCB at a 90-min incubation time.

FIG. 10A-C show representative spectral data of VH032 (1). Specifically, a ¹H NMR spectrum (FIG. 10A), a ¹³C NMR spectrum (FIG. 10B), and a high-resolution mass spectrum (FIG. 10C) are shown.

FIG. 11A-C show representative spectral data of VH032 (5). Specifically, a ¹H NMR spectrum (FIG. 11A), a ¹³C NMR spectrum (FIG. 11B), and a high-resolution mass spectrum (FIG. 11C) are shown.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “sample” means any biological matter that contains or potentially contains a von Hippel-Lindau protein (pVHL), purified or partially purified pVHL, recombinant pVHL with a naturally or non-naturally occurring sequence, or chimeric pVHL containing structural elements from multiple pVHL species. Thus, the term sample encompasses, but is not limited to, formulated product, cells, crude, fractionated or partially purified cell lysates (e.g., engineered to include a recombinant nucleic acid encoding a pVHL), and a solution (e.g., a buffer solution). It is further understood that the term sample encompasses tissue samples, including, without limitation, mammalian tissue samples, livestock tissue samples (e.g., sheep, cow, and pig tissue samples), primate tissue samples, and human tissue samples.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “contacting” means bringing a disclosed compound into close proximity to a sample (e.g., solution, buffer, cell, protein, etc.). This may be accomplished by, for example, any conventional drug delivery techniques (e.g., any of the administration techniques detailed above) or, in the case of in vitro analysis, by, e.g., providing a disclosed compound to a well or culture media to which a sample is exposed.

As used herein, the terms “effective amount” and “amount effective” refer to an amount that is sufficient to achieve the desired result or to have an effect on an undesired condition. For example, in various aspects, an “effective amount” refers to an amount that is sufficient to achieve the desired fluorescence emission. The specific effective dose level for any particular sample will depend upon a variety of factors including the instrument being used; the assay being performed; the ligands being evaluated and like factors well known in the pharmacological arts. For example, it is well within the skill of the art to start doses of a compound at levels lower than those required to achieve the desired effect and to gradually increase the dosage until the desired effect is achieved. Guidance can be found in the literature for appropriate dosages for given classes of biological and pharmacological probes and for given assays.

The term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage form can comprise a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsiloxane), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The terms “von Hippel-Lindau ligand,” “von Hippel-Lindau protein ligand,” “VHL ligand,” and “pVHL ligand” are used interchangeably, and refer to a molecule, e.g., a small molecule, that is capable of binding to von Hippel-Lindau protein. For example, a VHL ligand can be capable of binding reversibly or non-reversibly to von Hippel-Lindau protein. Examples of VHL ligands include, but are not limited to, VH032, VH298, MZ1, VH032, Me-VH032 amine, BOC-VH032, VH032 Phenol, and VH032-PEG4-amine. Additional VHL ligands and residues thereof are disclosed herein. See also Galdeano, et al. (2014) Journal of Medicinal Chemistry 57: 8657-8663 and Soares, et al. (2018) Journal of Medicinal Chemistry 61: 599-618.

The terms “non-von Hippel-Lindau ligand,” “non-von Hippel-Lindau protein ligand,” “non-VHL ligand,” and “non-pVHL ligand” are used interchangeably, and refer to a molecule, e.g., a small molecule, that does not bind to von Hippel-Lindau protein. Examples of non-VHL ligands include, but are not limited to, (+)-JQ-1, thalidomide 4′-oxyacetamido-alkylC4-amine, and dBET1.

The term “pharmaceutically acceptable” describes a material that is not biologically or otherwise undesirable, i.e., without causing an unacceptable level of undesirable biological effects or interacting in a deleterious manner.

As used herein, the term “fluorophore” is a fluorescent chemical compound that is capable of re-emitting light upon excitation. In various aspects, the fluorophore is intended to serve as a molecular probe for in vitro observation. Examples of fluorophores include, but are not limited to, coumarin based dyes (e.g., hydroxycoumarin, aminocoumarin, methoxycoumarin), fluorescein based dyes (e.g., fluorescein and carboxyfluorescein), SO₃-based conjugated systems (e.g., Alexa Fluor® 350, Alexa Fluor® 405, Alexa Fluor® 488, Alexa Fluor® 532, Alexa Fluor® 546, Alexa Fluor® 555, Alexa Fluor® 568, Alexa Fluor® 594, Alexa Fluor® 647, Alexa Fluor® 680, Alexa Fluor® 750, Texas Red®, Cy®5), boron systems (e.g., Bodipy®), and tetramethylrhodamine.

The term “antibody” is used in the broadest sense, and includes monoclonal antibodies, polyclonal antibodies, multispecific antibodies (including bispecific antibodies), antibody fragments that can bind an antigen (including, Fab′, F(ab)₂, Fv, single chain antibodies, diabodies), and recombinant peptides comprising the foregoing, as long as they exhibit the desired biological activity and antigen binding specificity. Examples of antibodies include, but are limited to Tb-anti-GST, Tb-anti-HIS, Tb-anti-FLAG, Tb-anti-HA, Eu-anti-GST, Eu-anti-HIS, Eu-anti-FLAG, and Eu-anti-HA.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. In a further aspect, the alkyl group can be substituted. For example, the alkyl group can be substituted with one or more groups including, but not limited to, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. In a further aspect, the cycloalkyl group and heterocycloalkyl group can be substituted. For example, the cycloalkyl group and heterocycloalkyl group can be substituted with 0, 1, 2, 3, or 4 groups independently selected from C1-C4 alkyl, C3-C7 cycloalkyl, C1-C4 alkoxy, —NH₂, (C1-C4) alkylamino, (C1-C4)(C1-C4) dialkylamino, ether, halogen, —OH, C1-C4 hydroxyalkyl, —NO₂, silyl, sulfo-oxo, —SH, and C1-C4 thioalkyl, as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. In a further aspect, the alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. In a further aspect, the cycloalkenyl group and heterocycloalkenyl group can be substituted. For example, the cycloalkenyl group and heterocycloalkenyl group can be substituted with 0, 1, 2, 3, or 4 groups independently selected from C1-C4 alkyl, C3-C7 cycloalkyl, C1-C4 alkoxy, C2-C4 alkenyl, C3-C6 cycloalkenyl, C2-C4 alkynyl, aryl, heteroaryl, aldehyde, —NH₂, (C1-C4) alkylamino, (C1-C4)(C1-C4) dialkylamino, carboxylic acid, ester, ether, halogen, —OH, C1-C4 hydroxyalkyl, ketone, azide, —NO₂, silyl, sulfo-oxo, —SH, and C1-C4 thioalkyl, as described herein.

The term “alkynyl” as used herein is a hydrocarbon group of 2 to 24 carbon atoms with a structural formula containing at least one carbon-carbon triple bond. In a further aspect, the alkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptenyl, cyclooctynyl, cyclononenyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. In a further aspect, the cycloalkynyl group and heterocycloalkynyl group can be substituted. For example, the cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. In a further aspect, the aryl group can be substituted. For example, the aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” or “CO” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. In a further aspect, the heteroaryl group can be substituted. For example, the heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxy” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN or —C≡N.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄ OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄ OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched)alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon-containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

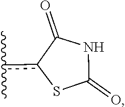

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

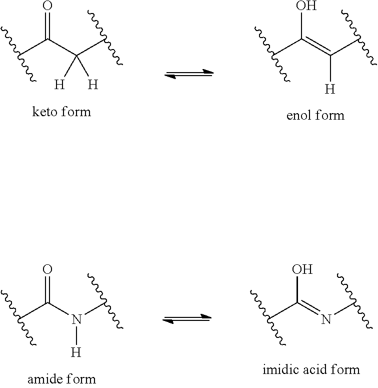 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

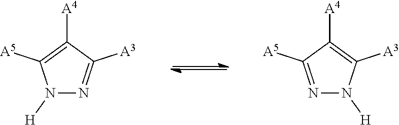

Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

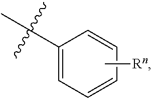

which is understood to be equivalent to a formula:

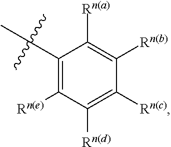

wherein n is typically an integer. That is, R n is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOUNDS

In one aspect, disclosed are compounds useful in modulating von Hippel-Lindau protein (pVHL) and in identifying pVHL ligands. As further detailed herein, pVHL ligands can be useful in, for example, treating or preventing anti-chronic anemia and anti-chronic ischemia, and also as proteolysis targeting chimeras (PROTACS) to degrade proteins for various therapeutic applications.

In one aspect, the compounds of the invention are useful in modulating pVHL, as further described herein.

In one aspect, the compounds of the invention are useful in identifying pVHL ligands, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

1. Structure

In one aspect, disclosed are compounds having a structure represented by a formula:

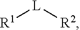

wherein L is a linker; wherein R¹ is a residue of a fluorophore, a residue of biotin, or a residue of a biotin derivative; and wherein R² is a residue of a von Hippel-Lindau protein (pVHL) ligand.

In a further aspect, the residue of the pVHL ligand has a structure represented by a formula:

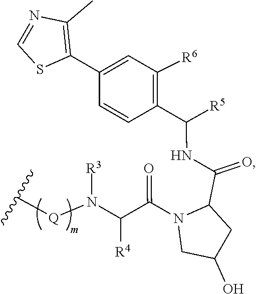

wherein m is 0 or 1; wherein Q, when present, is —OC(O)—, —C(R^{10a})(R^{10b})C(O)—, —OC(R^{10a})(R^{10b})C(O)—, —C(R^{10a}(R^{10b})C(O)C(cyclopropyl)C(O)—, —C(R^{10a}(R^{10b})C(O)N(R^{11a})CH₂CH(R^{11b})C(O)—, —C(C3-C4 cycloalkyl)C(O)—, —NH(CH₂CH₂O)_{q}CH₂C(O)—, —NHCH₂C(cyclopropyl)C(O)—, or —CH₂C(O)N(R¹²)CH(R¹³)C(O)—; wherein q, when present, is 1, 2, 3, 4, 5, or 6; wherein each of R^{10a} and R^{10b}, when present, is independently hydrogen or C1-C4 alkyl; or wherein each of R^{10a} and R^{10b}, when present, are covalently bound, and, together comprise a C3-C4 cycloalkyl or a C2-C3 heterocycloalkyl; or wherein R¹⁰, when present, is covalently bound to R³, and, together with the intermediate atoms, comprises a 5-membered heterocycle; wherein each of R^{11a} and R^{11b}, when present, are covalently bound, and, together with the intermediate atoms, comprise a 4-membered heterocycle; wherein R¹², when present, is hydrogen; and wherein R¹³, when present, is C1-C4 alkyl, —CH₂C₆H₅, or —C₆H₅; or wherein each of R¹² and R¹³, when present, are covalently bound, and, together with the intermediate atoms, comprise an 10-membered heterocycloalkyl; wherein R³ is hydrogen or C1-C4 alkyl; and wherein R⁴ is a C1-C4 alkyl, C1-C4 hydroxyalkyl, or C₆H₅; or wherein each of R³ and R⁴ are covalently bound, and, together with the intermediate atoms, comprise a 5- or 6-membered heterocycle having 0 or 1 —OH group; or wherein each of R³ and R¹⁰, when present, are covalently bound, and, together with the intermediate atoms, comprise a 5-membered heterocycle; wherein R⁵ is hydrogen or methyl; and wherein R⁶ is hydrogen, —OH, or C1-C4 alkyl halide.

In a further aspect, L is selected from C2-C15 alkyl and —(CH₂CH₂O)ₙ, wherein n is selected from 1, 2, 3, 4, 5, 6, 7, and 8.

In a further aspect, each of R³, R⁵, and R⁶ is hydrogen.

In a further aspect, the compound has a structure represented by a formula:

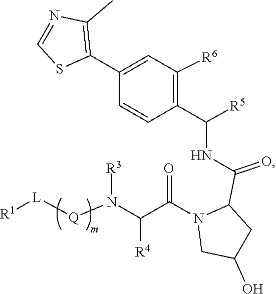

wherein m is 0 or 1; wherein Q, when present, is —OC(O)—, —C(R^{10a})(R^{10b})C(O)—, —OC(R^{10a})(R^{10b})C(O)—, —C(R^{10a})(R^{10b})C(O)C(cyclopropyl)C(O)—, —C(R^{10a})(R^{10b})C(O)N(R^{11a})CH₂CH(R^{11b})C(O)—, —C(C3-C4 cycloalkyl)C(O)—, —NH(CH₂CH₂O)_{q}CH₂C(O)—, —NHCH₂C(cyclopropyl)C(O)—, or —CH₂C(O)N(R¹²)CH(R¹³)C(O)—; wherein q, when present, is 1, 2, 3, 4, 5, or 6; wherein each of R^{10a} and R^{10b}, when present, is independently hydrogen or C1-C4 alkyl; or wherein each of R^{10a} and R^{10b}, when present, are covalently bound, and, together comprise a C3-C4 cycloalkyl or a C2-C3 heterocycloalkyl; or wherein R¹⁰, when present, is covalently bound to R³, and, together with the intermediate atoms, comprises a 5-membered heterocycle; wherein each of R^{11a} and R^{11b}, when present, are covalently bound, and, together with the intermediate atoms, comprise a 4-membered heterocycle; wherein R¹², when present, is hydrogen; and wherein R¹³, when present, is C1-C4 alkyl, —CH₂C₆H₅, or —C₆H₅; or wherein each of R¹² and R¹³, when present, are covalently bound, and, together with the intermediate atoms, comprise an 10-membered heterocycloalkyl; wherein R¹ is a 4,4-difluoro-4-bora-3a,4a-diaza-s-indacene (BODIPY) fluorophore; wherein R³ is hydrogen or C1-C4 alkyl; and wherein R⁴ is a C1-C4 alkyl, C1-C4 hydroxyalkyl, or C₆H₅; or wherein each of R³ and R⁴ are covalently bound, and, together with the intermediate atoms, comprise a 5- or 6-membered heterocycle having 0 or 1 —OH group; or wherein each of R³ and R¹⁰, when present, are covalently bound, and, together with the intermediate atoms, comprise a 5-membered heterocycle; wherein R⁵ is hydrogen or methyl; and wherein R⁶ is hydrogen, —OH, or C1-C4 alkyl halide.

In a further aspect, the compound has a structure represented by a formula:

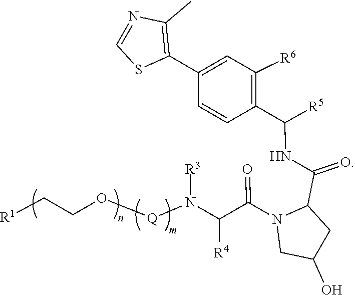

In a further aspect, the compound has a structure represented by a formula:

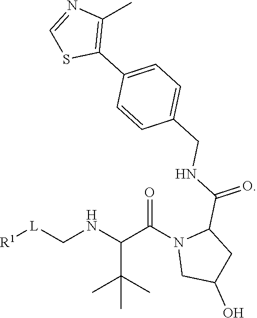

In a further aspect, the compound has a structure represented by a formula:

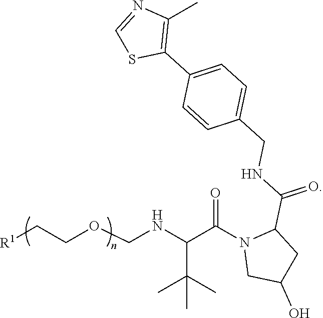

In a further aspect, the compound has a structure represented by a formula:

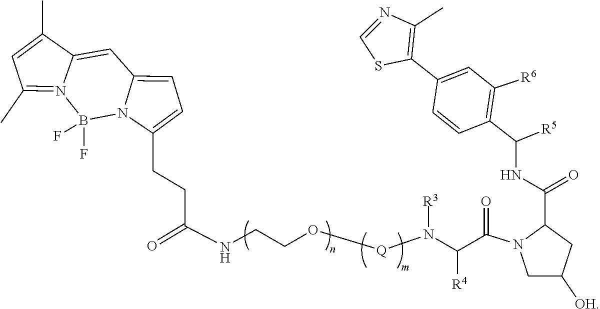

In a further aspect, the compound has a structure represented by a formula:

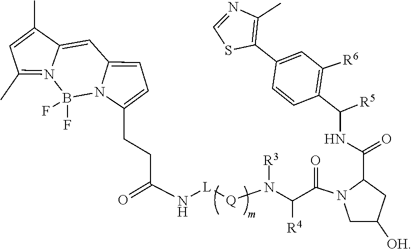

In a further aspect, the compound is:

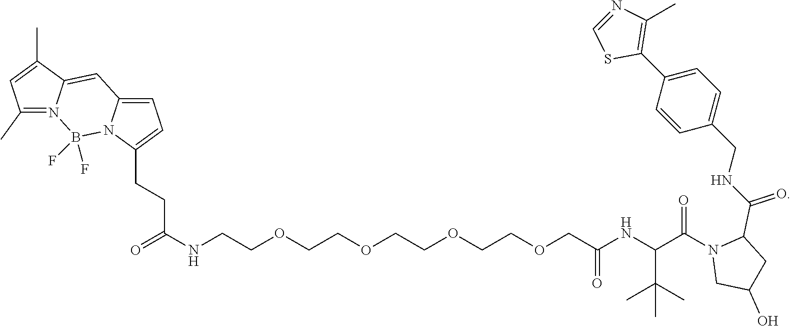

In a further aspect, the compound is:

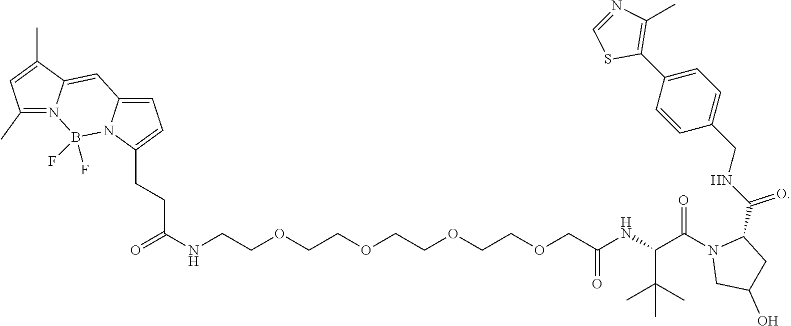

In one aspect, m is 0 or 1. In a further aspect, m is 0. In a still further aspect, m is 1.

In one aspect, n is selected from 1, 2, 3, 4, 5, 6, 7, and 8. In a further aspect, n is selected from 1, 2, 3, 4, 5, 6, and 7. In a still further aspect, n is selected from 1, 2, 3, 4, 5, and 6. In yet a further aspect, n is selected from 1, 2, 3, 4, and 5. In an even further aspect, n is selected from 1, 2, 3, and 4. In a still further aspect, n is selected from 1, 2, and 3. In yet a further aspect, n is selected from 1 and 2. In an even further aspect, n is selected from 2, 3, 4, 5, 6, 7, and 8. In a still further aspect, n is selected from 3, 4, 5, 6, 7, and 8. In yet a further aspect, n is selected from 4, 5, 6, 7, and 8. In an even further aspect, n is selected from 5, 6, 7, and 8. In a still further aspect, n is selected from 6, 7, and 8. In yet a further aspect, n is selected from 7 and 8.

In one aspect, q, when present, is 1, 2, 3, 4, 5, or 6. In a further aspect, q, when present, is 1, 2, 3, 4, or 5. In a still further aspect, q, when present, is 1, 2, 3, or 4. In yet a further aspect, q, when present, is 1, 2, or 3. In an even further aspect, q, when present, is 1 or 2. In a still further aspect, q, when present, is 2, 3, 4, 5, or 6. In yet a further aspect, q, when present, is 3, 4, 5, or 6. In an even further aspect, q, when present, is 4, 5, or 6. In a still further aspect, q, when present, is 5 or 6.

a. L Groups

In one aspect, L is a linker. Examples of linkers include, but are not limited to, C2-C15 alkyl and —(CH₂CH₂O)ₙ.

In a further aspect, L is C2-C15 alkyl. In a still further aspect, L is C2-C15 alkyl. In yet a further aspect, L is C2-C8 alkyl. In an even further aspect, L is C5 alkyl.

In a further aspect, L is —(CH₂CH₂O)ₙ—. In a still further aspect, L is —(CH₂CH₂O)₄—.

b. Q Groups

In one aspect, Q, when present, is —OC(O)—, —C(R^{10a})(R^{10b})C(O)—, —OC(R^{10a})(R^{10b})C(O)—, —C(R^{10a})(R^{10b})C(O)C(cyclopropyl)C(O)—, —C(R^{10a})(R^{10b})C(O)N(R^{11a})CH₂CH(R^{11b})C(O)—, —C(C3-C4 cycloalkyl)C(O)—, —NH(CH₂CH₂O)_{q}CH₂C(O)—, —NHCH₂C(cyclopropyl)C(O)—, or —CH₂C(O)N(R¹²)CH(R¹³)C(O)—.

In a further aspect, Q, when present, is —OC(O)— or —OC(R^{10a})(R^{10b})C(O)—. In a still further aspect, Q, when present, is —OC(O)—. In yet a further aspect, Q, when present, is —OC(R^{10a})(R^{10b})C(O)—.

In a further aspect, Q, when present, is —C(R^{10a})(R^{10b})C(O)—, —C(R^{10a})(R^{10b})C(O)C(cyclopropyl)C(O)—, —C(R^{10a})(R^{10b})C(O)N(R^{11a})CH₂CH(R^{11b})C(O)—, —C(C3-C4 cycloalkyl)C(O)—, or —CH₂C(O)N(R¹²)CH(R¹³)C(O)—. In a still further aspect, Q, when present, is —C(R^{10a})(R^{11b})C(O)—, —C(R^{10a})(R^{10b})C(O)C(cyclopropyl)C(O)—, or —C(C3-C4 cycloalkyl)C(O)—. In yet a further aspect, Q, when present, is —C(R^{10a})(R^{11b})C(O)— or —C(R^{10a})(R^{10b})C(O)C(cyclopropyl)C(O)—. In an even further aspect, Q, when present, is —C(R^{10a})(R^{10b})C(O)C(cyclopropyl)C(O)— or —C(C3-C4 cycloalkyl)C(O)—. In a still further aspect, Q, when present, is —C(R^{10a})(R^{10b})C(O)— or —C(C3-C4 cycloalkyl)C(O)—. In yet a further aspect, Q, when present, is —C(R^{10a})(R^{10b})C(O)—. In an even further aspect, Q, when present, is —C(R^{10a})(R^{10b})C(O)C(cyclopropyl)C(O)—. In a still further aspect, Q, when present, is —C(C3-C4 cycloalkyl)C(O)—.

In a further aspect, Q, when present, is —C(R^{10a})(R^{10b})C(O)N(R^{11a})CH₂CH(R^{11b})C(O)— or —CH₂C(O)N(R¹²)CH(R¹³)C(O)—. In a still further aspect, Q, when present, is —C(R^{10a})(R^{10b})C(O)N(R^{11a})CH₂CH(R^{11b})C(O)—. In yet a further aspect, Q, when present, is —CH₂C(O)N(R¹²)CH(R¹³)C(O)—.

In a further aspect, Q, when present, is —NH(CH₂CH₂O)_{q}CH₂C(O)— or —NHCH₂C(cyclopropyl)C(O)—. In a still further aspect, Q, when present, is —NH(CH₂CH₂O)_{q}CH₂C(O)—. In yet a further aspect, Q, when present, is —NHCH₂C(cyclopropyl)C(O)—.

C. R¹ Groups

In one aspect, R¹ is a residue of a fluorophore, a residue of biotin, or a residue of a biotin derivative.

In a further aspect, R¹ is a residue of biotin or a residue of a biotin derivative. In a still further aspect, R¹ is a residue of biotin. In yet a further aspect, R¹ is a residue of a biotin derivative. Examples of biotin derivatives include, but are not limited to, biocytin and desthiobiotin.

In a further aspect, R¹ is a residue of a fluorophore. Examples of fluorophores include, but are not limited to, fluorescein, Oregon green, rhodamine (e.g., TAMRA dye), eosin, Texas red, indocarbocyanine, oxacarbocyanine, thiacarbocyanine, merocyanine, a squaraine derivative, a naphthalene derivative (e.g., a dansyl or prodan derivative), a coumarin derivative, an oxadiazole derivative (e.g., pyridyloxazole, nitrobenzoxadiazole, benzoxadiazole), an anthracene derivative (e.g., an anthraquinone such as DRAQ5, DRAQ7, and CyTRAK Orange), cascade blue, Nile red, Nile blue, cresyl violate, oxazine 170, proflavin, acridine orange, acridine yellow, auramine, crystal violet, malachite green, prophin, phthalocyanine, an alexa fluor series dye, bilirubin, and 4,4-difluoro-4-bora-3a,4a-diaza-s-indacene (BODIPY) fluorophores.

In a further aspect, the fluorophore is a 4,4-difluoro-4-bora-3a,4a-diaza-s-indacene (BODIPY) fluorophore. In a still further aspect, the BODIPY fluorophore is selected from:

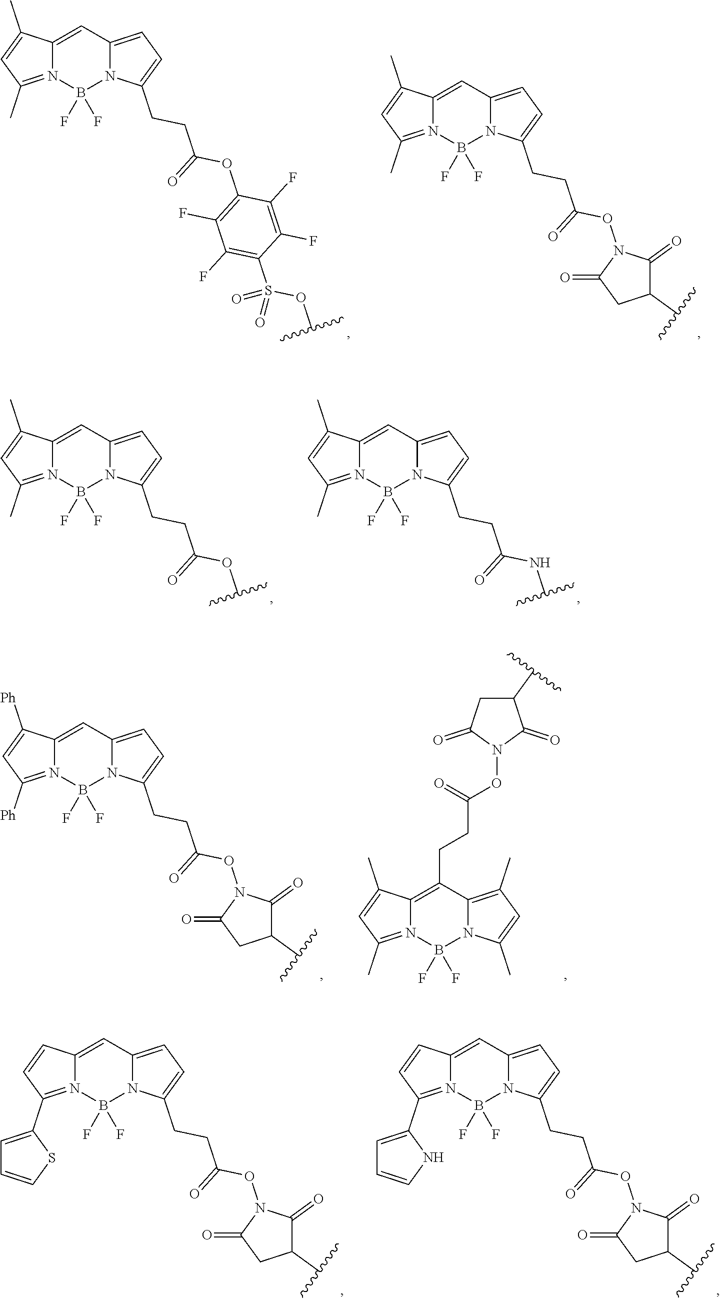

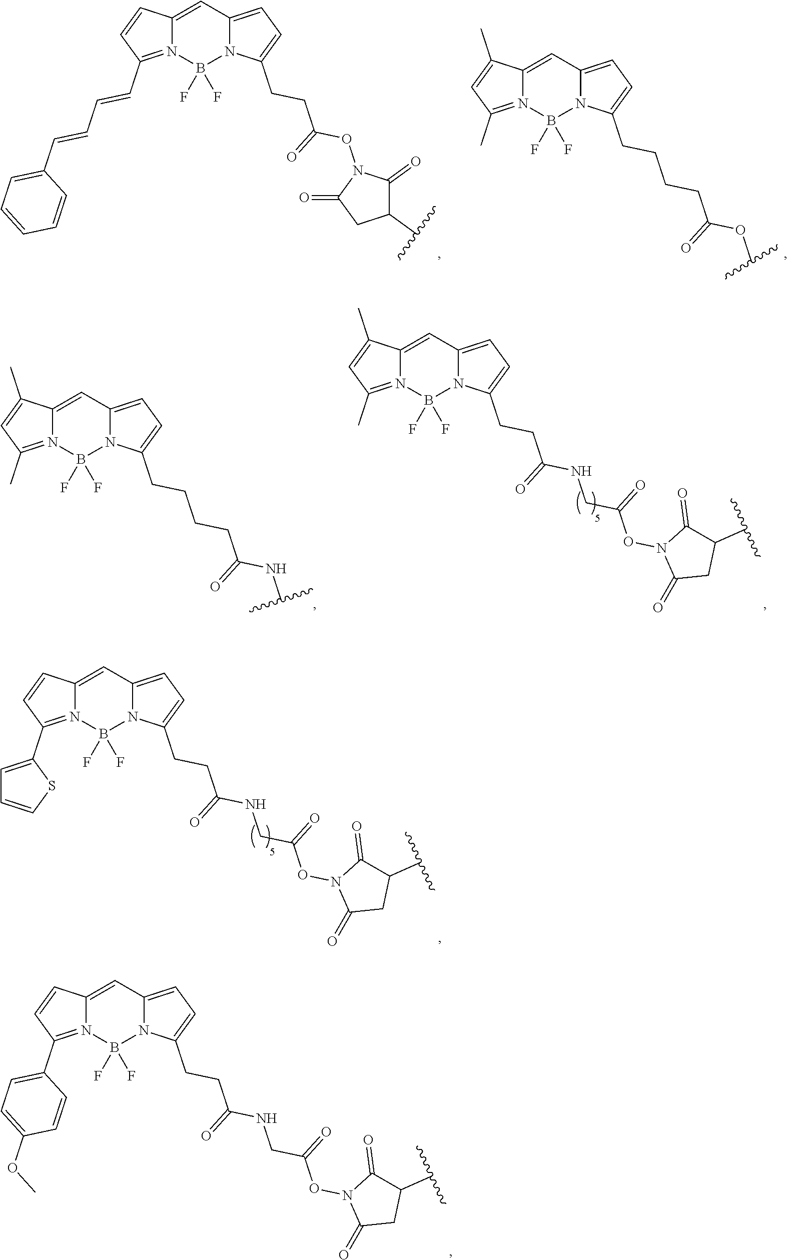

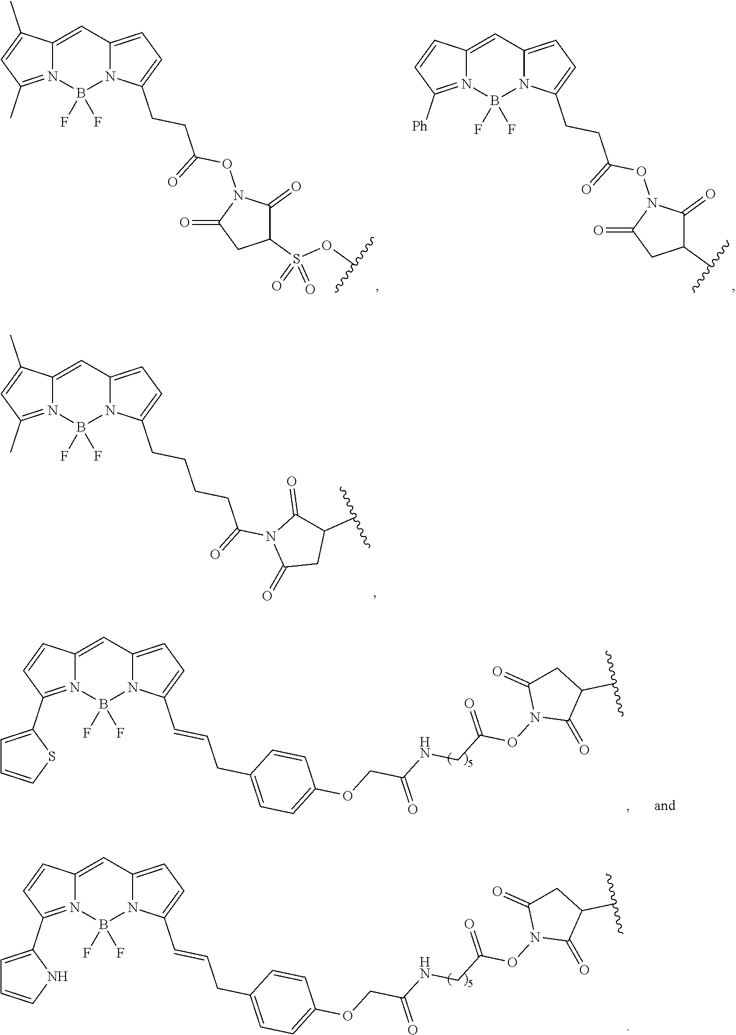

In yet a further aspect, the BODIPY fluorophore is:

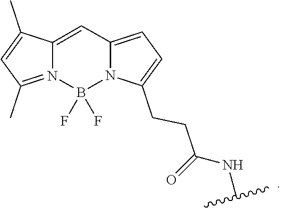

d. R² Groups

In one aspect, R² is a residue of a von Hippel-Lindau protein (pVHL) ligand.

In a further aspect, the residue of the pVHL ligand has a structure represented by a formula:

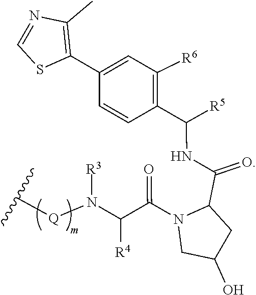

In a further aspect, the residue of the pVHL ligand has a structure represented by a formula:

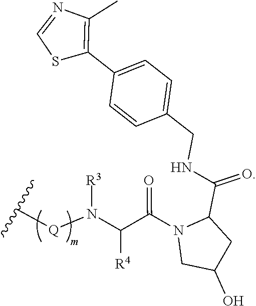

In a further aspect, the residue of the pVHL ligand has a structure represented by a formula:

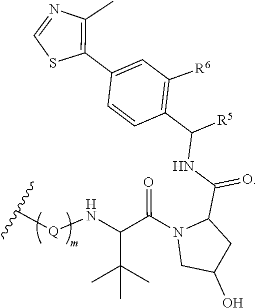

In a further aspect, the residue of the pVHL ligand has a structure represented by a formula:

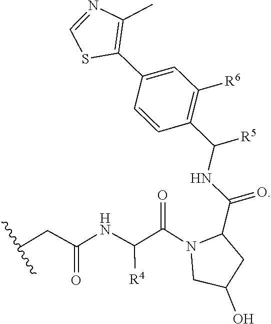

In a further aspect, the residue of the pVHL ligand has a structure selected from:

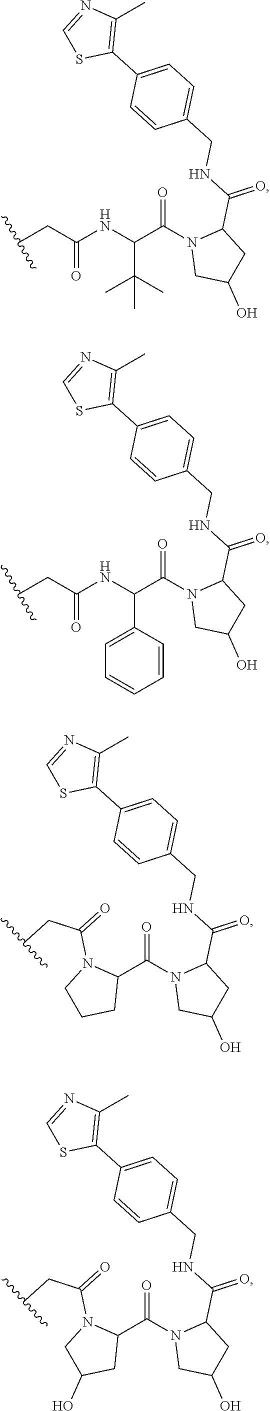

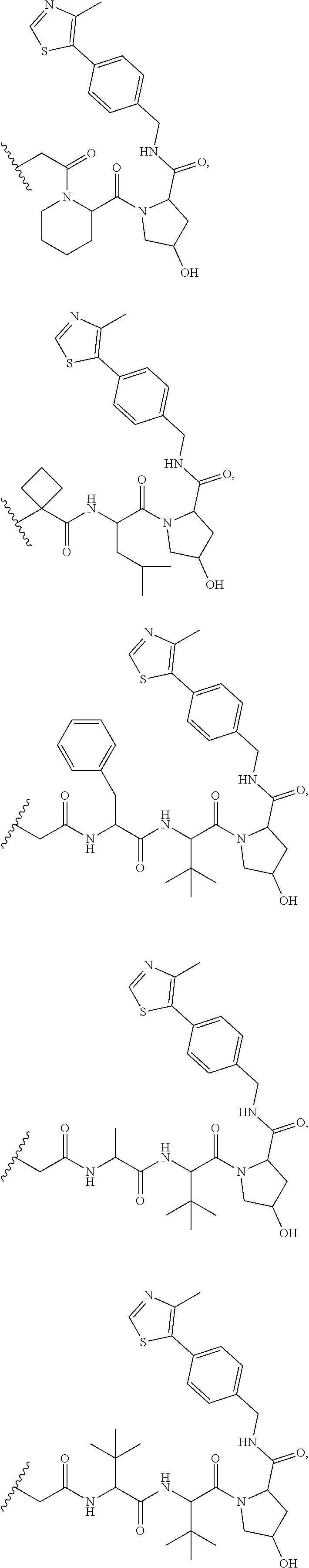

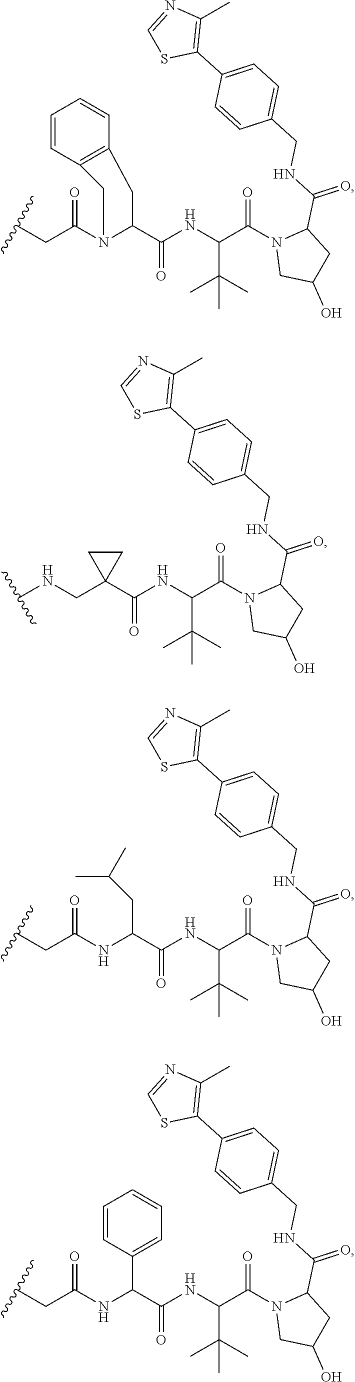 [SMILES: *CC(=O)N1Cc2ccccc2CC1C(=O)NC(C(=O)N1CC(O)CC1C(=O)NCc1ccc(-c2scnc2C)cc1)C(C)(C)C ; *NCC1(C(=O)NC(C(=O)N2CC(O)CC2C(=O)NCc2ccc(-c3scnc3C)cc2)C(C)(C)C)CC1 ; *CC(=O)NC(CC(C)C)C(=O)NC(C(=O)N1CC(O)CC1C(=O)NCc1ccc(-c2scnc2C)cc1)C(C)(C)C ; *CC(=O)NC(C(=O)NC(C(=O)N1CC(O)CC1C(=O)NCc1ccc(-c2scnc2C)cc1)C(C)(C)C)c1ccccc1]

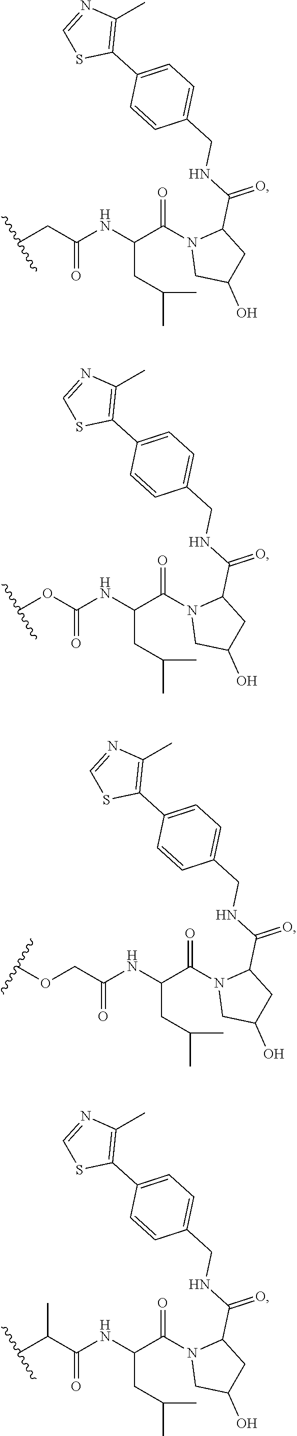 [SMILES: *CC(=O)NC(CC(C)C)C(=O)N1CC(O)CC1C(=O)NCc1ccc(-c2scnc2C)cc1 ; *OC(=O)NC(CC(C)C)C(=O)N1CC(O)CC1C(=O)NCc1ccc(-c2scnc2C)cc1 ; *OCC(=O)NC(CC(C)C)C(=O)N1CC(O)CC1C(=O)NCc1ccc(-c2scnc2C)cc1 ; *C(C)C(=O)NC(CC(C)C)C(=O)N1CC(O)CC1C(=O)NCc1ccc(-c2scnc2C)cc1]

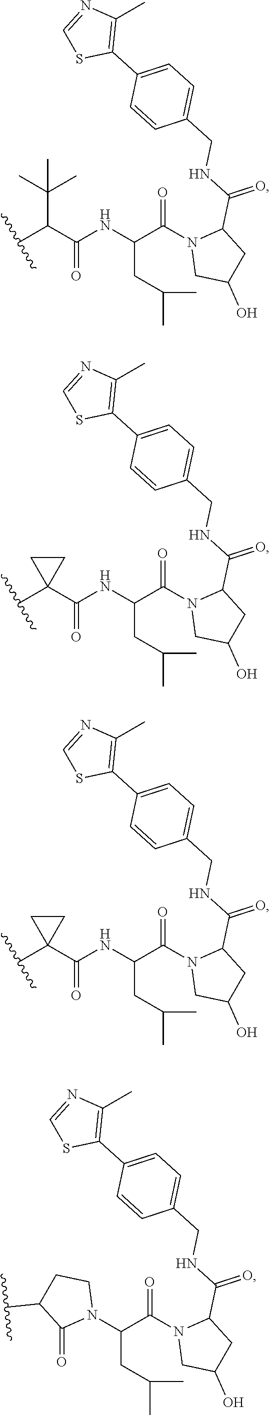

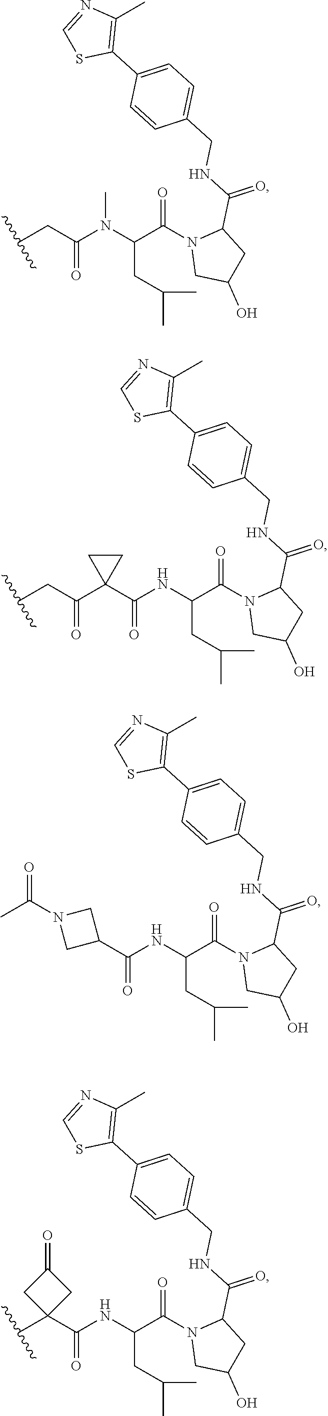

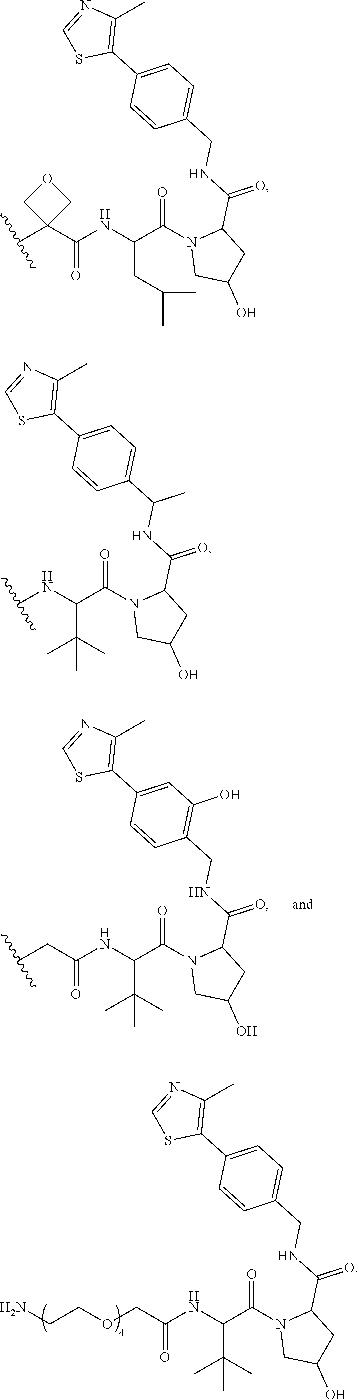

In a further aspect, the residue of the pVHL ligand has a structure:

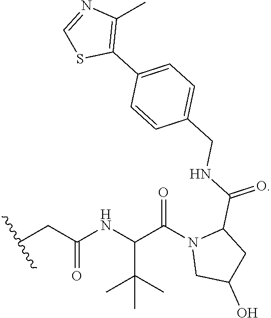 [SMILES: *CC(=O)NC(C(=O)N1CC(O)CC1C(=O)NCc1ccc(-c2scnc2C)cc1)C(C)(C)C]

e. R³ and R⁴ Groups

In one aspect, R³ is hydrogen or C1-C4 alkyl; and wherein R⁴ is a C1-C4 alkyl, C1-C4 hydroxyalkyl, or C₆H₅; or wherein each of R³ and R⁴ are covalently bound, and, together with the intermediate atoms, comprise a 5- or 6-membered heterocycle having 0 or 1 —OH group; or wherein each of R³ and R^{10a}, when present, are covalently bound, and, together with the intermediate atoms, comprise a 5-membered heterocycle.

In a further aspect, R³ is hydrogen or C1-C4 alkyl. In a still further aspect, R³ is hydrogen, methyl, ethyl, n-propyl, or isopropyl. In yet a further aspect, R³ is hydrogen, methyl, or ethyl. In an even further aspect, R³ is hydrogen or ethyl. In a still further aspect, R³ is hydrogen or methyl.

In a further aspect, R³ is C1-C4 alkyl. In a still further aspect, R³ is methyl, ethyl, n-propyl, or isopropyl. In yet a further aspect, R³ is methyl or ethyl. In an even further aspect, R³ is ethyl. In a still further aspect, R³ is methyl.

In a further aspect, R³ is hydrogen.

In a further aspect, R⁴ is a C1-C4 alkyl, C1-C4 hydroxyalkyl, or C₆H₅. In a still further aspect, R⁴ is methyl, ethyl, n-propyl, isopropyl, —CH₂OH, —CH₂CH₂OH, —CH₂CH₂CH₂OH, —CH(CH₃)CH₂OH, or C₆H₅. In yet a further aspect, R⁴ is methyl, ethyl, —CH₂OH, —CH₂CH₂OH, or C₆H₅. In an even further aspect, R⁴ is methyl, —CH₂OH, or C₆H₅.

In a further aspect, R⁴ is a C1-C4 alkyl. In a still further aspect, R⁴ is methyl, ethyl, n-propyl, or isopropyl. In yet a further aspect, R⁴ is methyl or ethyl. In an even further aspect, R⁴ is methyl. In a still further aspect, R⁴ is a C4 alkyl. In yet a further aspect, R⁴ is isobutyl, sec-butyl, or tert-butyl. In an even further aspect, R⁴ is tert-butyl.

In a further aspect, R⁴ is a C1-C4 hydroxyalkyl. In a still further aspect, R⁴ is —CH₂OH, —CH₂CH₂OH, —CH₂CH₂CH₂OH, or —CH(CH₃)CH₂OH. In yet a further aspect, R⁴ is —CH₂OH or —CH₂CH₂OH. In an even further aspect, R⁴ is —CH₂OH.

In a further aspect, R⁴ is C₆H₅.

In a further aspect, each of R³ and R⁴ are covalently bound, and, together with the intermediate atoms, comprise a 5- or 6-membered heterocycle having 0 or 1 —OH group. Examples of 5- and 6-membered heterocycles include, but are not limited to, pyrrolidinyl, pyrrolidino, piperidinyl, piperidino, piperazinyl, piperazino, morpholinyl, morpholino, thiomorpholinyl, thiomorpholino, tetrahydrofuranyl, tetrahydrothiofuranyl, tetrahydropyranyl, and pyranyl. In a still further aspect, each of R³ and R⁴ are covalently bound, and, together with the intermediate atoms, comprise a 5- or 6-membered heterocycle having 1 —OH group. In yet a further aspect, each of R³ and R⁴ are covalently bound, and, together with the intermediate atoms, comprise a 5- or 6-membered heterocycle having 0 —OH groups. In an even further aspect, each of R³ and R⁴ are covalently bound, and, together with the intermediate atoms, comprise an unsubstituted 5- or 6-membered heterocycle.

In a further aspect, each of R³ and R⁴ are covalently bound, and, together with the intermediate atoms, comprise a 5-membered heterocycle having 0 or 1 —OH group. In a still further aspect, each of R³ and R⁴ are covalently bound, and, together with the intermediate atoms, comprise a 5-membered heterocycle having 1 —OH group. In yet a further aspect, each of R³ and R⁴ are covalently bound, and, together with the intermediate atoms, comprise a 5-membered heterocycle having 0 —OH groups.

In a further aspect, each of R³ and R⁴ are covalently bound, and, together with the intermediate atoms, comprise a 6-membered heterocycle having 0 or 1 —OH group. In a still further aspect, each of R³ and R⁴ are covalently bound, and, together with the intermediate atoms, comprise a 6-membered heterocycle having 1 —OH group. In yet a further aspect, each of R³ and R⁴ are covalently bound, and, together with the intermediate atoms, comprise a 6-membered heterocycle having 0 —OH groups.

In a further aspect, each of R³ and R^{10a}, when present, are covalently bound, and, together with the intermediate atoms, comprise a 5-membered heterocycle. Examples of heterocycles include, but are not limited to, pyrrolidinyl, pyrrolidino, thiolanyl, and tetrahydrofuranyl. In a still further aspect, each of R³ and R^{10a}, when present, are covalently bound, and, together with the intermediate atoms, comprise an unsubstituted 5-membered heterocycle.

f. R⁵ Groups

In one aspect, R⁵ is hydrogen or methyl. In a further aspect, R⁵ is hydrogen. In a still further aspect, R⁵ is methyl.

g. R⁶ Groups

In one aspect, R⁶ is hydrogen, —OH, or C1-C4 alkyl halide. In a further aspect, R⁶ is hydrogen, —OH, —CH₂F, —CH₂Cl, —CH₂CH₂F, —CH₂CH₂Cl, —CH₂CH₂CH₂F, —CH₂CH₂CH₂Cl, —CH(CH₃)CH₂F, or —CH(CH₃)CH₂Cl. In a still further aspect, R⁶ is hydrogen, —OH, —CH₂F, —CH₂Cl, —CH₂CH₂F, or —CH₂CH₂Cl. In yet a further aspect, R⁶ is hydrogen, —OH, —CH₂F, or —CH₂Cl.

In a further aspect, R⁶ is —OH.

In a further aspect, R⁶ is C1-C4 alkyl halide. In a still further aspect, R⁶ is —CH₂F, —CH₂Cl, —CH₂Br, —CH₂CH₂F, —CH₂CH₂Cl, —CH₂CH₂Br, —CH₂CH₂CH₂F, —CH₂CH₂CH₂Cl, —CH₂CH₂CH₂Br, —CH(CH₃)CH₂F, —CH(CH₃)CH₂Cl, or —CH(CH₃)CH₂Br. In yet a further aspect, R⁶ is —CH₂F, —CH₂Cl, —CH₂Br, —CH₂CH₂F, —CH₂CH₂Cl, or —CH₂CH₂Br. In an even further aspect, R⁶ is —CH₂F, —CH₂Cl, or —CH₂Br.

In a further aspect, R⁶ is hydrogen.

h. R^{10A} and R^{10B} Groups

In one aspect, each of R^{10a} and R^{10b}, when present, is independently hydrogen or C1-C4 alkyl; or wherein each of R^{10a} and R^{10b}, when present, are covalently bound, and, together comprise a C3-C4 cycloalkyl or a C2-C3 heterocycloalkyl; or wherein R^{10a}, when present, is covalently bound to R³, and, together with the intermediate atoms, comprises a 5-membered heterocycle.

In a further aspect, each of R^{10a} and R^{10b}, when present, is independently hydrogen or C1-C4 alkyl. In a still further aspect, each of R^{10a} and R^{10b}, when present, is independently hydrogen, methyl, ethyl, n-propyl, or isopropyl. In yet a further aspect, each of R^{10a} and R^{10b}, when present, is independently hydrogen, methyl, or ethyl. In an even further aspect, each of R^{10a} and R^{10b}, when present, is independently hydrogen or methyl.

In a further aspect, each of R^{10a} and R^{10b}, when present, is independently C1-C4 alkyl. In a still further aspect, each of R^{10a} and R^{10b}, when present, is independently methyl, ethyl, n-propyl, or isopropyl. In yet a further aspect, each of R^{10a} and R^{10b}, when present, is independently methyl or ethyl. In an even further aspect, each of R^{10a} and R^{10b}, when present, is methyl.

In a further aspect, each of R^{10a} and R^{10b}, when present, is hydrogen.

In a further aspect, each of R^{10a} and R^{10b}, when present, are covalently bound, and, together comprise a C3-C4 cycloalkyl or a C2-C3 heterocycloalkyl. In a still further aspect, each of R^{10a} and R^{10b}, when present, are covalently bound, and, together comprise a C3-C4 cycloalkyl or a C2-C3 heterocycloalkyl, and are unsubstituted.

In a further aspect, each of R^{10a} and R^{10b}, when present, are covalently bound, and, together comprise a C3-C4 cycloalkyl. In a still further aspect, each of R^{10a} and R^{10b}, when present, are covalently bound, and, together comprise a cyclopropyl. In yet a further aspect, each of R^{10a} and R^{10b}, when present, are covalently bound, and, together comprise a cyclobutyl. In an even further aspect, each of R^{10a} and R^{10b}, when present, are covalently bound, and, together comprise an unsubstituted C3-C4 cycloalkyl.

In a further aspect, each of R^{10a} and R^{10b}, when present, are covalently bound, and, together comprise a C2-C3 heterocycloalkyl. Examples of C2-C3 heterocycloalkyls include, but are not limited to, oxirane, aziridine, and thiirane. In a still further aspect, each of R^{10a} and R^{10b}, when present, are covalently bound, and, together comprise an unsubstituted C2-C3 heterocycloalkyl.

In a further aspect, R^{10a}, when present, is covalently bound to R³, and, together with the intermediate atoms, comprises a 5-membered heterocycle. Examples of 5-membered heterocycles include, but are not limited to, pyrrolidinyl, pyrrolidino, thiolanyl, and tetrahydrofuranyl. In a still further aspect, R^{10a}, when present, is covalently bound to R³, and, together with the intermediate atoms, comprises an unsubstituted 5-membered heterocycle.

i. R^{11A} and R^{11B} Groups

In one aspect, each of R^{11a} and R^{11b}, when present, are covalently bound, and, together with the intermediate atoms, comprise a 4-membered heterocycle. Examples of 4-membered heterocycles include, but are not limited to, trimethylene oxide, thietane, 1,3-diazetidine, and azetidine. In a further aspect, each of R^{11a} and R^{11b}, when present, are covalently bound, and, together with the intermediate atoms, comprise an unsubstituted 4-membered heterocycle.

j. R¹² and R¹³ Groups

In one aspect, R¹², when present, is hydrogen; and wherein R¹³, when present, is C1-C4 alkyl, —CH₂C₆H₅, or —C₆H₅; or wherein each of R¹² and R¹³, when present, are covalently bound, and, together with the intermediate atoms, comprise a 10-membered heterocycloalkyl.

In a further aspect, R¹², when present, is hydrogen.

In a further aspect, R¹³, when present, is C1-C4 alkyl, —CH₂C₆H₅, or —C₆H₅. In a still further aspect, R¹³, when present, is methyl, ethyl, n-propyl, isopropyl, —CH₂C₆H₅, or —C₆H₅. In yet a further aspect, R¹³, when present, is methyl, ethyl, —CH₂C₆H₅, or —C₆H₅. In an even further aspect, R¹³, when present, is methyl, —CH₂C₆H₅, or —C₆H₅.

In a further aspect, R¹³, when present, is C1-C4 alkyl. In a still further aspect, R¹³, when present, is methyl, ethyl, n-propyl, or isopropyl. In yet a further aspect, R¹³, when present, is methyl or ethyl. In an even further aspect, R¹³, when present, is methyl.

In a further aspect, R¹³, when present, is —CH₂C₆H₅ or —C₆H₅. In a still further aspect, R¹³, when present, is —CH₂C₆H₅. In yet a further aspect, R¹³, when present, is —C₆H₅.

In a further aspect, each of R¹² and R¹³, when present, are covalently bound, and, together with the intermediate atoms, comprise a 10-membered heterocycloalkyl. Examples of 10-membered heterocycloalkyls include, but are not limited to, tetrahydroisoquinolinyl and decahydroisoquinolinyl.

2. Example Compounds

In one aspect, a compound can be present as:

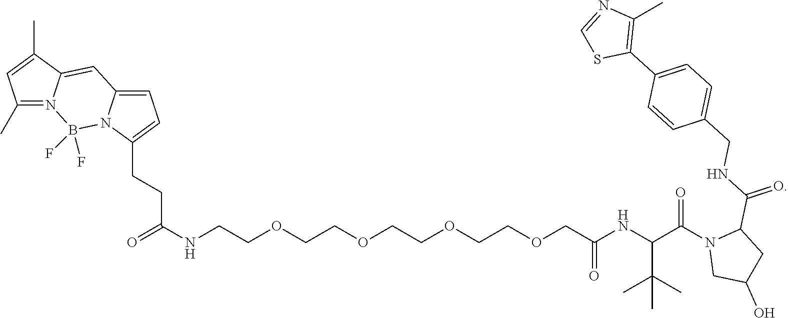

In one aspect, a compound can be present as:

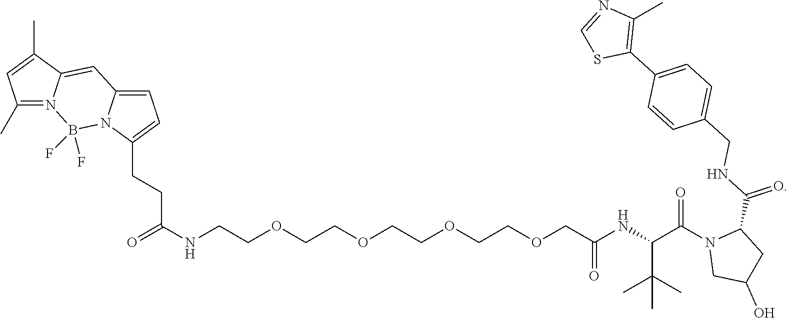

### C. METHODS OF MAKING A COMPOUND

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-III, as described and exemplified below. The following examples are provided so that the invention might be more fully understood, are illustrative only, and should not be construed as limiting.

1. Route I

In one aspect, the compounds disclosed herein can be prepared as shown below.

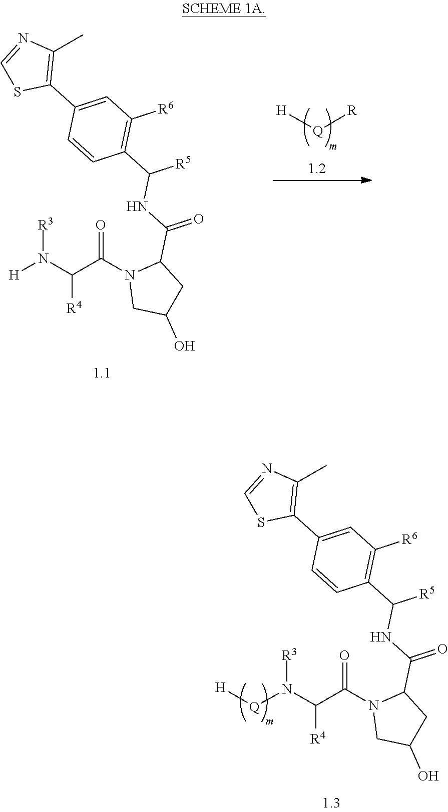

Compounds are represented in generic form, where R is —OH, —NH₂, or —O— acetate such that 1.2 is a carboxylic acid, amide, or anhydride, and with other substituents as noted in compound descriptions elsewhere herein. A more specific example is set forth below.

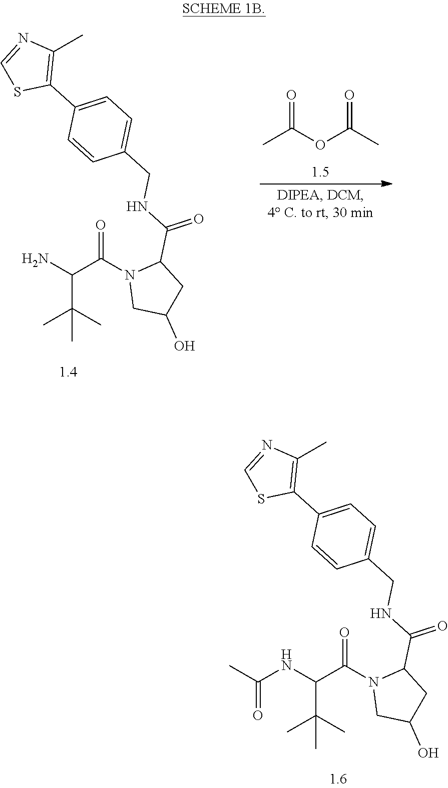

In one aspect, compounds of type 1.6, and similar compounds, can be prepared according to reaction Scheme 1B above. Thus, compounds of type 1.6 can be prepared by a coupling reaction between an appropriate amine, e.g., 1.4 as shown above, and an appropriate carboxylic acid, amide, or anhydride, e.g., 1.5 as shown above. Appropriate amines and appropriate carboxylic acids, amines, and anhydrides are commercially available or prepared by methods known to one skilled in the art. The coupling reaction is carried out in the presence of an appropriate base, e.g., diisopropylethylamine (DIPEA), in an appropriate solvent, e.g., dichloromethane, at an appropriate temperature, e.g., 4° C. to room temperature, for an appropriate period of time, e.g., 30 minutes. As can be appreciated by one skilled in the art, the above reaction provides an example of a generalized approach wherein compounds similar in structure to the specific reactants above (compounds similar to compounds of type 1.1 and 1.2), can be substituted in the reaction to provide pVHL ligands similar to Formula 1.3.

2. Route II

In one aspect, the compounds disclosed herein can be prepared as shown below.

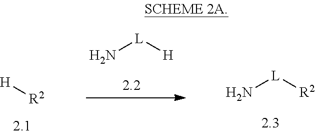

Compounds are represented in generic form, with substituents as noted in compound descriptions elsewhere herein. A more specific example is set forth below.

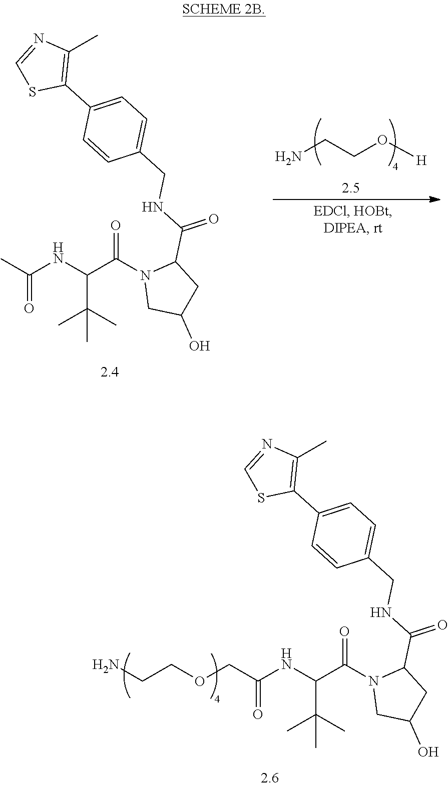

In one aspect, compounds of type 2.6, and similar compounds, can be prepared according to reaction Scheme 2B above. Thus, compounds of type 2.6 can be prepared by a coupling reaction between an appropriate carboxyl or amine analog, e.g., 2.4 as shown above, and an appropriate alcohol or amine, e.g., 2.5 as shown above. Appropriate amines and appropriate alcohols are commercially available or prepared by methods known to one skilled in the art. The coupling reaction is carried out in the presence of an appropriate coupling agent, e.g., hydroxybenzotriazole (HOBt), an appropriate activating agent, e.g., 1-ethyl-3-(3-dimethylaminopropyl)carbodiimide (EDCI), and an appropriate base, e.g., diisopropylethylamine (DIPEA), at an appropriate temperature, e.g., room temperature. As can be appreciated by one skilled in the art, the above reaction provides an example of a generalized approach wherein compounds similar in structure to the specific reactants above (compounds similar to compounds of type 2.1 and 2.2), can be substituted in the reaction to provide compounds similar to Formula 2.6.

3. Route III

In one aspect, the compounds disclosed herein can be prepared as shown below.

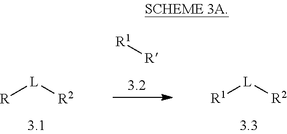

Compounds are represented in generic form, where R and R′ are independently groups capable of coupling with one another such as, for example, carboxylic acids and amines, and with other substituents as noted in compound descriptions elsewhere herein. A more specific example is set forth below.

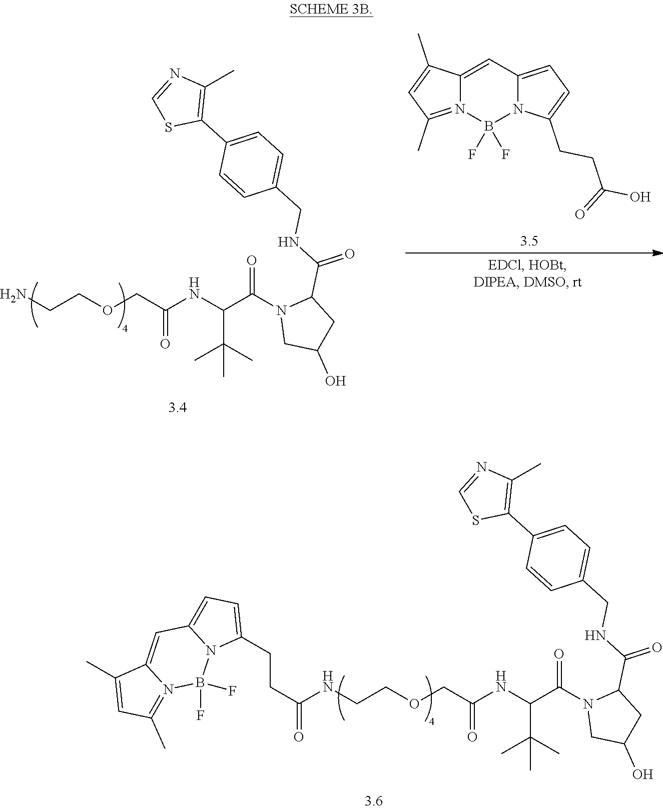

In one aspect, compounds of type 3.6, and similar compounds, can be prepared according to reaction Scheme 3B above. Thus, compounds of type 3.6 can be prepared by a coupling reaction between an appropriate alcohol or amine analog, e.g., 3.4 as shown above, and an appropriate carboxylic acid, e.g., 3.5 as shown above. Appropriate carboxylic acids are commercially available or prepared by methods known to one skilled in the art. The coupling reaction is carried out in the presence of an appropriate coupling agent, e.g., hydroxybenzotriazole (HOBt), an appropriate activating agent, e.g., 1-ethyl-3-(3-dimethylaminopropyl)carbodiimide (EDCI), and an appropriate base, e.g., diisopropylethylamine (DIPEA), in an appropriate solvent, e.g., dimethylsulfoxide, at an appropriate temperature, e.g., room temperature. As can be appreciated by one skilled in the art, the above reaction provides an example of a generalized approach wherein compounds similar in structure to the specific reactants above (compounds similar to compounds of type 3.1 and 3.2), can be substituted in the reaction to provide compounds similar to Formula 3.6.

### D. METHODS OF MODULATING VON HIPPEL-LINDAU PROTEIN IN A SAMPLE

In one aspect, disclosed are methods of modulating von Hippel-Lindau protein (pVHL) in a sample, the method comprising contacting the sample with an effective amount of a disclosed compound, thereby modulating VHL protein in the sample.

In various aspects, disclosed are methods of modulating von Hippel-Lindau protein (pVHL) in a sample, the method comprising contacting the sample with an effective amount of a compound, having a structure represented by a formula:

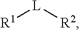

wherein L is a linker; wherein R¹ is a residue of a fluorophore, a residue of biotin, or a residue of a biotin derivative; and wherein R² is a residue of a von Hippel-Lindau protein (pVHL) ligand, thereby modulating VHL protein in the sample.

In various aspects, the compound has a structure represented by a formula:

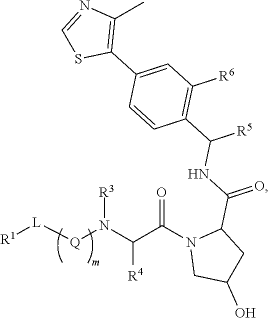

wherein m is 0 or 1; wherein Q, when present, is —OC(O)—, —C(R^{10a})(R^{10b})C(O)—, —OC(R^{10a})(R^{10b})C(O)—, —C(R^{10a})(R^{10b})C(O)C(cyclopropyl)C(O)—, —C(R^{10a})(R^{10b})C(O)N(R^{11a})CH₂CH(R^{11b})C(O)—, —C(C3-C4 cycloalkyl)C(O)—, —NH(CH₂CH₂O)_{q}CH₂C(O)—, —NHCH₂C(cyclopropyl)C(O)—, or —CH₂C(O)N(R¹²)CH(R¹³)C(O)—; wherein q, when present, is 1, 2, 3, 4, 5, or 6; wherein each of R^{10a} and R^{10b}, when present, is independently hydrogen or C1-C4 alkyl; or wherein each of R^{10a} and R^{10b}, when present, are covalently bound, and, together comprise a C3-C4 cycloalkyl or a C2-C3 heterocycloalkyl; or wherein R¹⁰, when present, is covalently bound to R³, and, together with the intermediate atoms, comprises a 5-membered heterocycle; wherein each of R^{11a} and R^{11b}, when present, are covalently bound, and, together with the intermediate atoms, comprise a 4-membered heterocycle; wherein R¹², when present, is hydrogen; and wherein R¹³, when present, is C1-C4 alkyl, —CH₂C₆H₅, or —C₆H₅; or wherein each of R¹² and R¹³, when present, are covalently bound, and, together with the intermediate atoms, comprise an heterocycloalkyl; wherein R¹ is a 4,4-difluoro-4-bora-3a,4a-diaza-s-indacene (BODIPY) fluorophore; wherein R³ is hydrogen or C1-C4 alkyl; and wherein R⁴ is a C1-C4 alkyl, C1-C4 hydroxyalkyl, or C₆H₅; or wherein each of R³ and R⁴ are covalently bound, and, together with the intermediate atoms, comprise a 5- or 6-membered heterocycle having 0 or 1 —OH group; or wherein each of R³ and R¹⁰, when present, are covalently bound, and, together with the intermediate atoms, comprise a 5-membered heterocycle; wherein R⁵ is hydrogen or methyl; and wherein R⁶ is hydrogen, —OH, or C1-C4 alkyl halide.

In a further aspect, the compound has a structure represented by a formula:

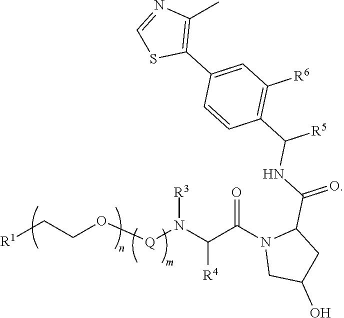

In a further aspect, the compound has a structure represented by a formula:

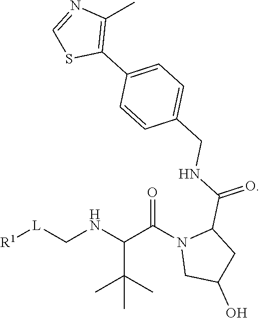

In a further aspect, the compound has a structure represented by a formula:

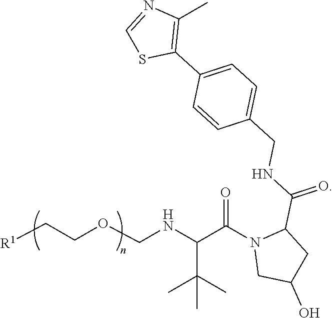

In a further aspect, the compound has a structure represented by a formula:

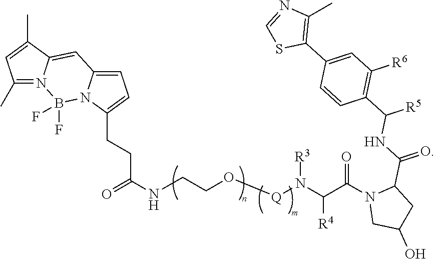

In a further aspect, the compound has a structure represented by a formula:

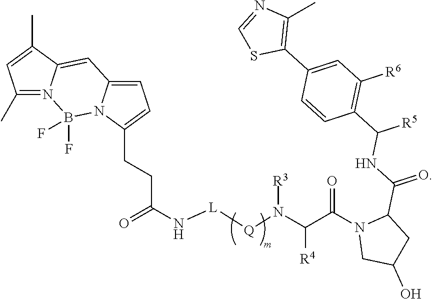

In a further aspect, the compound is:

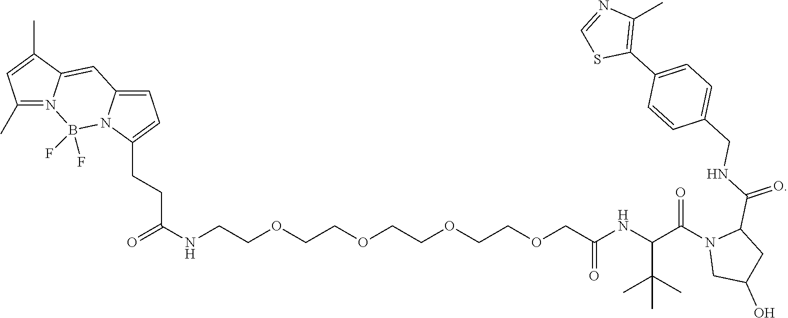

In a further aspect, the compound is:

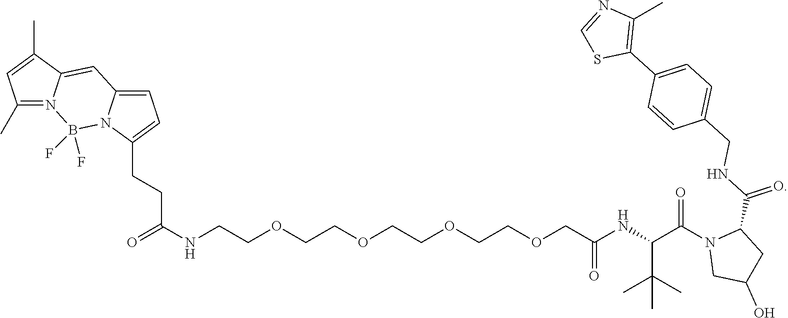

In various aspects, modulating is decreasing. In various further aspects, modulating is inhibiting.

In various aspects, contacting is in the presence of an antibody. As would be appreciated by one of ordinary skill in the art, the antibody selected can be dependent on the assay being used. Exemplary antibodies are well-known by those of ordinary skill, and include, for example, Tb-anti-GST, Tb-anti-HIS, Tb-anti-FLAG, Tb-anti-HA, Eu-anti-GST, Eu-anti-HIS, Eu-anti-FLAG, and Eu-anti-HA.

In various aspects, contacting is in the presence of a pVHL ligand. Examples of pVHL ligands include, but are not limited to, VH032, VH298, MZ1, VH032, Me-VH032 amine, BOC-VH032, VH032 Phenol, and VH032-PEG4-amine. Additional VHL ligands and residues thereof are disclosed herein. See also Galdeano, et al. (2014) Journal of Medicinal Chemistry 57: 8657-8663 and Soares, et al. (2018) Journal of Medicinal Chemistry 61: 599-618.

In various aspects, contacting is in the presence of a non-pVHL ligand. Examples of non-VHL ligands include, but are not limited to, (+)-JQ-1, thalidomide 4′-oxyacetamido-alkylC4-amine, and dBET1.

In various aspects, contacting is for a time period of from about 90 minutes to about 300 minutes, from about 90 minutes to about 250 minutes, from about 90 minutes to about 200 minutes, from about 90 minutes to about 150 minutes, from about 90 minutes to about 100 minutes, from about 10 minutes to about 300 minutes, from about 150 minutes to about 300 minutes, from about 200 minutes to about 300 minutes, from about 250 minutes to about 300 minutes, from about 100 minutes to about 250 minutes, or from about 150 minutes to about 200 minutes.

In various aspects, the sample is a buffer. In a further aspect, the sample is a cell. In a still further aspect, the cell is mammalian.

In various aspects, the effective amount is within ±30% of a K_{d} concentration of the compound. In a further aspect, the effective amount is within ±25% of a K_{d} concentration of the compound. In a still further aspect, the effective amount is within ±20% of a K_{d} concentration of the compound.

In various aspects, the K_{d} concentration is of from about 2.0 nM to about 5.0 nM, from about 2.0 nM to about 4.5 nM, from about 2.0 nM to about 4.0 nM, from about 2.0 nM to about 3.5 nM, from about 2.0 nM to about 3.0 nM, from about 2.0 nM to about 2.5 nM, from about 2.5 nM to about 5.0 nM, from about 3.0 nM to about 5.0 nM, from about 3.5 nM to about 5.0 nM, from about 4.0 nM to about 5.0 nM, from about 4.0 nM to about 5.0 nM, or from about 2.5 nM to about 4.5 nM. In a further aspect, the K_{d} concentration is about 3.0 nM.

In various aspects, the effective concentration is of from about 2.0 nM to about 5.0 nM, from about 2.0 nM to about 4.5 nM, from about 2.0 nM to about 4.0 nM, from about 2.0 nM to about 3.5 nM, from about 2.0 nM to about 3.0 nM, from about 2.0 nM to about 2.5 nM, from about 2.5 nM to about 5.0 nM, from about 3.0 nM to about 5.0 nM, from about 3.5 nM to about 5.0 nM, from about 4.0 nM to about 5.0 nM, from about 4.0 nM to about 5.0 nM, or from about 2.5 nM to about 4.5 nM. In a further aspect, the effective amount concentration is about 4.0 nM.

### E. METHODS OF IDENTIFYING A VON HIPPEL-LINDAU PROTEIN (PVHL) LIGAND

In one aspect, disclosed are methods of identifying a von Hippel-Lindau protein (pVHL) ligand in a library, the method comprising: (a) providing a library that contains a plurality of ligands; (b) combining a disclosed compound and a sample having pVHL, thereby forming a mixture; (c) exposing each ligand to the mixture; and (d) detecting a fluorescence emission of the mixture after exposure to each ligand, wherein a decrease in fluorescence emission indicates that the ligand is a pVHL ligand, and wherein a lack of decrease in fluorescence emission indicates that the ligand is a non-pVHL ligand.

In various aspects, disclosed are methods of identifying a von Hippel-Lindau protein (pVHL) ligand in a library, the method comprising: (a) providing a library that contains a plurality of ligands; (b) combining a compound and a sample having pVHL, thereby forming a mixture; (c) exposing each ligand to the mixture; and (d) detecting a fluorescence emission of the mixture after exposure to each ligand, wherein a decrease in fluorescence emission indicates that the ligand is a pVHL ligand, wherein a lack of decrease in fluorescence emission indicates that the ligand is a non-pVHL ligand, and wherein the compound has a structure represented by a formula:

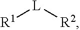

wherein L is a linker; wherein R¹ is a residue of a fluorophore, a residue of biotin, or a residue of a biotin derivative; and wherein R² is a residue of a von Hippel-Lindau protein (pVHL) ligand, thereby modulating VHL protein in the sample.

In various aspects, the compound has a structure represented by a formula:

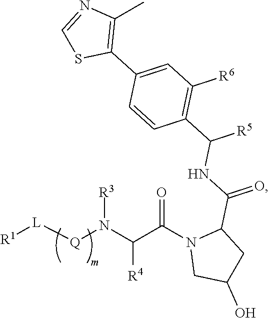

wherein m is 0 or 1; wherein Q, when present, is —OC(O)—, —C(R^{10a})(R^{10b})C(O)—, —OC(R^{10a})(R^{10b})C(O)—, —C(R^{10a})(R^{10b})C(O)C(cyclopropyl)C(O)—, —C(R^{10a})(R^{10b})C(O)N(R^{11a})CH₂CH(R^{11b})C(O)—, —C(C3-C4 cycloalkyl)C(O)—, —NH(CH₂CH₂O)_{q}CH₂C(O)—, —NHCH₂C(cyclopropyl)C(O)—, or —CH₂C(O)N(R¹²)CH(R¹³)C(O)—; wherein q, when present, is 1, 2, 3, 4, 5, or 6; wherein each of R^{10a} and R^{10b}, when present, is independently hydrogen or C1-C4 alkyl; or wherein each of R^{10a} and R^{10b}, when present, are covalently bound, and, together comprise a C3-C4 cycloalkyl or a C2-C3 heterocycloalkyl; or wherein R¹⁰, when present, is covalently bound to R³, and, together with the intermediate atoms, comprises a 5-membered heterocycle; wherein each of R^{11a} and R^{11b}, when present, are covalently bound, and, together with the intermediate atoms, comprise a 4-membered heterocycle; wherein R¹², when present, is hydrogen; and wherein R¹³, when present, is C1-C4 alkyl, —CH₂C₆H₅, or —C₆H₅; or wherein each of R¹² and R¹³, when present, are covalently bound, and, together with the intermediate atoms, comprise an 10-membered heterocycloalkyl; wherein R¹ is a 4,4-difluoro-4-bora-3a,4a-diaza-s-indacene (BODIPY) fluorophore; wherein R³ is hydrogen or C1-C4 alkyl; and wherein R⁴ is a C1-C4 alkyl, C1-C4 hydroxyalkyl, or C₆H₅; or wherein each of R³ and R⁴ are covalently bound, and, together with the intermediate atoms, comprise a 5- or 6-membered heterocycle having 0 or 1 —OH group; or wherein each of R³ and R¹⁰, when present, are covalently bound, and, together with the intermediate atoms, comprise a 5-membered heterocycle; wherein R⁵ is hydrogen or methyl; and wherein R⁶ is hydrogen, —OH, or C1-C4 alkyl halide.

In a further aspect, the compound has a structure represented by a formula:

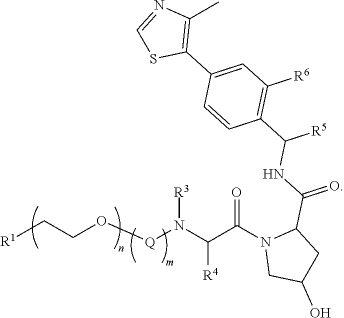

In a further aspect, the compound has a structure represented by a formula:

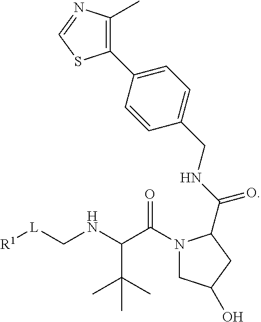

In a further aspect, the compound has a structure represented by a formula:

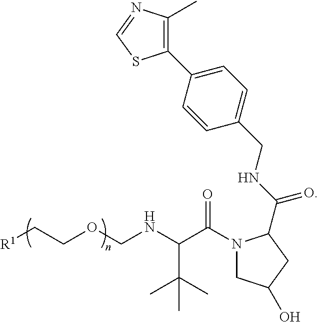

In a further aspect, the compound has a structure represented by a formula:

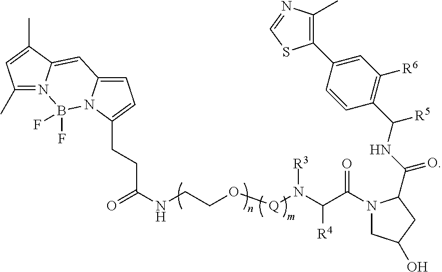

In a further aspect, the compound has a structure represented by a formula:

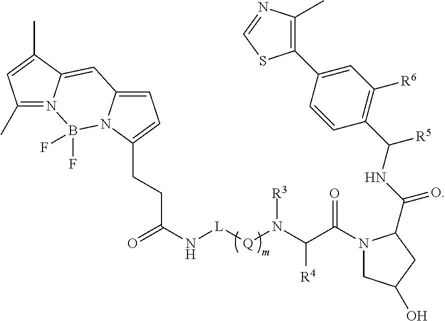

In a further aspect, the compound is:

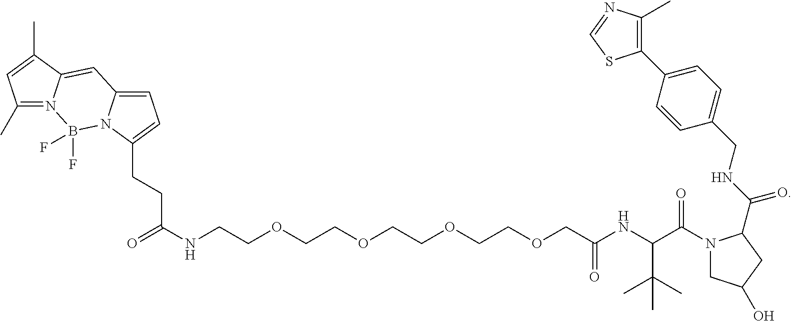

In a further aspect, the compound is:

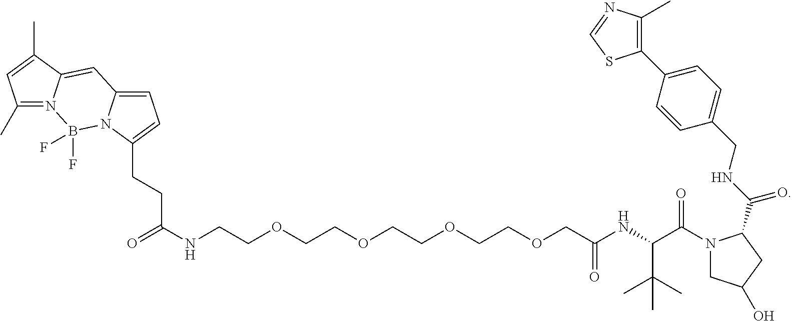

In various aspects, exposing is in the presence of an antibody. As would be appreciated by one of ordinary skill in the art, the antibody selected can be dependent on the assay being used. Exemplary antibodies are well-known by those of ordinary skill, and include, for example, Tb-anti-GST, Tb-anti-HIS, Tb-anti-FLAG, Tb-anti-HA, Eu-anti-GST, Eu-anti-HIS, Eu-anti-FLAG, and Eu-anti-HA.

In various aspects, the library contains at least one pVHL ligand. In a further aspect, the library contains a plurality of pVHL ligands. Examples of pVHL ligands include, but are not limited to, VH032, VH298, MZ1, VH032, Me-VH032 amine, BOC-VH032, VH032 Phenol, and VH032-PEG4-amine. Additional VHL ligands and residues thereof are disclosed herein. See also Galdeano, et al. (2014) Journal of Medicinal Chemistry 57: 8657-8663 and Soares, et al. (2018) Journal of Medicinal Chemistry 61: 599-618.

In various aspects, the library contains at least one non-pVHL ligand. In a further aspect, the library contains a plurality of non-pVHL ligands. Examples of non-VHL ligands include, but are not limited to, (+)-JQ-1, thalidomide 4′-oxyacetamido-alkylC4-amine, and dBET1.

In various aspects, exposing is for a time period of from about 90 minutes to about 300 minutes, from about 90 minutes to about 250 minutes, from about 90 minutes to about 200 minutes, from about 90 minutes to about 150 minutes, from about 90 minutes to about 100 minutes, from about 10 minutes to about 300 minutes, from about 150 minutes to about 300 minutes, from about 200 minutes to about 300 minutes, from about 250 minutes to about 300 minutes, from about 100 minutes to about 250 minutes, or from about 150 minutes to about 200 minutes.

### F. ADDITIONAL METHODS OF USING THE COMPOSITIONS

Provided are methods of using of a disclosed compound. In one aspect, the method of use is as a probe. The probe can be useful in, for example, identifying a von Hippel-Lindau protein ligand in a library. In a further aspect, the method of use is in modulating of von Hippel-Lindau protein in a sample such as, for example, a cell or a buffer. In a further aspect, the disclosed compounds can be used as single agents or in combination with one or more other probes, pVHL ligands, non-pVHL ligands, and antibodies in the aforementioned uses.

The samples, mixtures, and methods of the present invention can further comprise other agents as noted herein, which are usually applied in biological assays such as, for example, fluorescence assays.

1. Use of Compounds, Samples, and Mixtures

Also provided are the uses of the disclosed compounds, samples, and mixtures. Thus, in one aspect, the invention relates to the use of von Hippel-Lindau (VHL) small molecule probes.

In a further aspect, the use relates to a process for preparing a sample or mixture comprising an effective amount of a disclosed compound or a product of a disclosed method, and one or more of an antibody, a pVHL ligand, a non-pVHL ligand, a buffer, and a solvent, for use as in a fluorescence-based assay.

In a further aspect, the use relates to a process for preparing a sample or mixture comprising an effective amount of a disclosed compound or a product of a disclosed method, wherein one or more of an antibody, a pVHL ligand, a non-pVHL ligand, a buffer, and a solvent is intimately mixed with an effective amount of the disclosed compound or the product of a disclosed method.

In various aspects, the use relates to the modulation of von Hippel-Lindau protein in a sample. In a further aspect, the use relates to the modulation of von Hippel-Lindau protein in a buffer. In a further aspect, the use relates to the modulation of von Hippel-Lindau protein in a cell.

In various aspects, the use relates to the identification of a von Hippel-Lindau protein ligand in a library.

It is understood that the disclosed uses can be employed in connection with the disclosed compounds, methods, samples, mixtures, and kits. In a further aspect, the invention relates to the use of a disclosed compound, sample, or mixture for a fluorescence-based assay.

2. Kits

In one aspect, disclosed are kits comprising a disclosed compound, and one or more of: (a) a sample that contains von Hippel-Lindau protein (pVHL); (b) a library that contains a plurality of ligands; (c) instructions for modulating pVHL; (d) instructions for identifying a pVHL ligand and/or a non-pVHL ligand; and (e) instructions for performing a fluorescence-based assay.

In various aspects, the compounds, samples, and/or libraries described herein can be provided in a kit. The kit can also include combinations of the compounds, samples, and/or libraries.

In various aspects, the informational material can be descriptive, instructional, marketing or other material that relates to the methods described herein and/or to the use of the agents for the methods described herein. For example, the informational material may relate to the use of the compounds to modulate pVHL, to identify pVHL ligands and/or non-pVHL ligands, or to perform a fluorescence-based assay. The kits can also include paraphernalia for administering the compounds of this invention to a sample such as, for example, a buffer or a cell (e.g., in culture).

In various aspects, the compounds of the kit can include other ingredients, such as a solvent or buffer, a stabilizer, a preservative, a fragrance or other assay ingredient (e.g., an antibody). In such aspects, the kit can include instructions for admixing the compound and the other ingredients, or for using one or more compounds together with the other ingredients.

In a further aspect, the compound and the sample are co-formulated. In a still further aspect, the compound and the sample are co-packaged.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### G. EXAMPLES

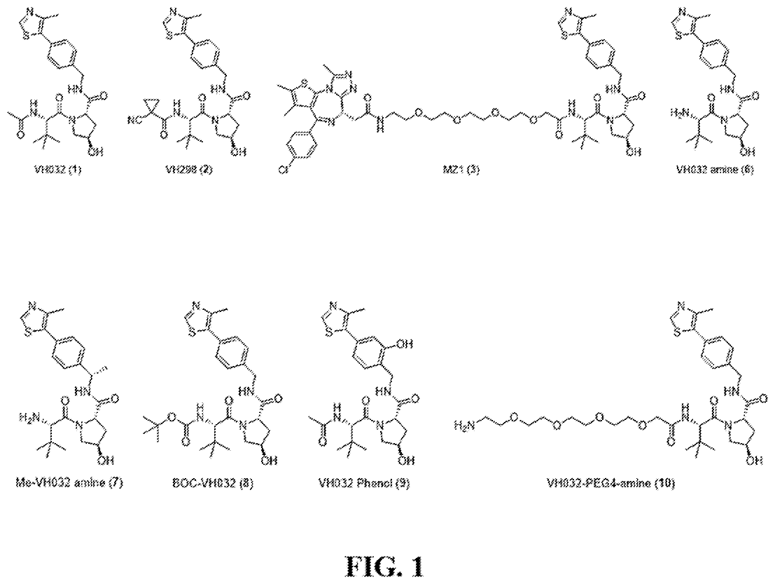

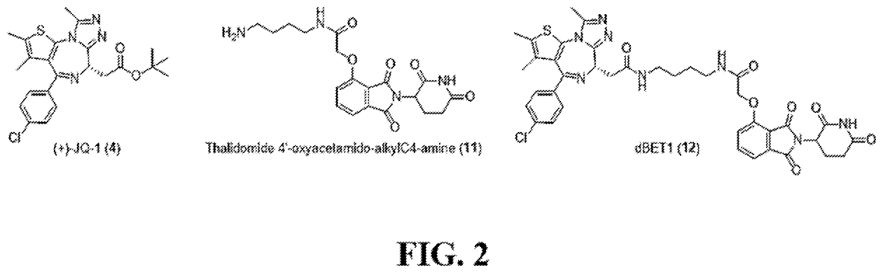

Herein, the first small molecule-based VHL fluorescent probe, BODIPY FL VH032 (5), is described, which is high affinity with a K_{d} value of 3.01 nM to a VCB protein complex in a TR-FRET binding assay. In a VHL FP assay, the BODIPY FL VH032 (5) has a K_{d} value of 100.8 nM to the VCB protein complex. Then, the newly developed BODIPY FL VH032 (5)-based TR-FRET and FP assays were used to test a panel of reported VHL ligands FIG. 1) including VH032 (1), VH298 (2), MZ1 (3), VH032 amine (6), Me-VH032 amine (7), BOC-VH032 (8), VH032 phenol (9), and VH032-PEG4-amine (10) (Gadd, et al. (2017) Structural basis of PROTAC cooperative recognition for selective protein degradation. Nat Chem Biol 13, 514-521; Soares, et al. (2018) Group-Based Optimization of Potent and Cell-Active Inhibitors of the von Hippel-Lindau (VHL) E3 Ubiquitin Ligase: Structure-Activity Relationships Leading to the Chemical Probe (2S,4R)-1-((S)-2-(1-Cyanocyclopropanecarboxamido)-3,3-dimethylbutanoyl)-4-hydroxy-N-(4-(4-methylthiazol-5-yl)benzyl)pyrrolidine-2-carboxamide (VH298). J Med Chem 61, 599-618; Lai, et al. (2016) Modular PROTAC Design for the Degradation of Oncogenic BCR-ABL. Angew Chem Int Ed Engl 55, 807-810; Raina, et al. (2016) PROTAC-induced BET protein degradation as a therapy for castration-resistant prostate cancer. Proc Natl Acad Sci USA 113, 7124-7129; Maniaci, et al. (2017) Homo-PROTACs: bivalent small-molecule dimerizers of the VHL E3 ubiquitin ligase to induce self-degradation. Nat Commun 8, 830) and non-VHL ligands (FIG. 2) including (+)-JQ1 (4), Thalidomide-4′-oxyacetamido-alkylC4-amine (11, a cereblon E3 ligase ligand), and dBET1 (12, a bivalent BRD-CRBN PROTAC) (Winter, et al. (2015) DRUG DEVELOPMENT. Phthalimide conjugation as a strategy for in vivo target protein degradation. Science 348, 1376-1381). Only VHL ligands showed binding, demonstrating the specificity of both the BODIPY FL VH032 (5)-mediated TR-FRET and FP assays. Whereas the BODIPY FL VH032 (5)-mediated FP assay has similar sensitivity to the reported FP assay based on a FAM-labeled HIF-1α peptide (FAM-DEALAHyp-YIPMDDDFQLRSF, 19-mer), the BODIPY FL VH032 (5)-mediated TR-FRET assay was more sensitive and consumed less VCB protein than the reported FP assay. In addition, The BODIPY FL VH032 (5)-mediated TR-FRET assay was resistant to assay interference and capable of detecting VHL ligands with a wide range of binding affinity. In summary, a new and high-affinity VHL fluorescent probe BODIPY FL VH032 (5) has been developed, and has been used develop a TR-FRET assay that is sensitive, selective, resistant to assay interference, and suitable for VHL ligand identification and characterization through large scale screening, as further described herein below.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

1. Chemistry Methods

VH032 amine and VH032-PEG4-amine hydrochloride salt were purchased from MedChemExpress LLC (Monmouth Junction, NJ). BODIPY FL propionic acid was purchased from BroadPharm (San Diego, CA). Acetic anhydride, N,N-diisopropyl ethylamine (DIPEA), dichloromethane (DCM), dimethyl sulfoxide (DMSO), 1-Ethyl-3-(3-dimethylaminopropyl)carbodiimide (EDCI), Hydroxybenzotriazole (HOBt) and all other basic chemical reagents and solvent were purchased from Sigma-Aldrich (St. Louis, MO). Dimethyl sulfoxide-d₆ (DMSO-d₆) and chloroform-d were purchased from Cambridge Isotope Laboratories, Inc. (Tewksbury, MA).

Reported protocols (Lin, et al. (2014) Development of BODIPY FL vindoline as a novel and high-affinity pregnane X receptor fluorescent probe. Bioconjug Chem 25, 1664-1677) were adopted to assess or verify reaction progress, product purity and identity; to determine high-resolution mass spectra and to record ¹H and ¹³C NMR spectra.

### a. (2S,4R)-1-((S)-2-acetamido-3,3-dimethylbutanoyl)-4-hydroxy-N-(4-(4-methylthiazol-5-yl)benzyl)pyrrolidine-2-carboxamide (1, VH032)

VH032 amine (6, 215 mg, 0.5 mmol) was solubilized in a stirred solution of DCM (5 mL) and DIPEA (263 μL, 1.5 mmol) with an ice-water batch. Ac₂O (60 μL, 0.635 mmol) was then added. The ice-water bath was removed after 5 min and the reaction was continued for another 25 min under room temperature (RT). The reaction mixture was diluted with DCM (20 mL) and quenched with brine (30 mL). After separation from the brine, the DCM solution was washed with brine (20 mL×2) and dried with anhydrous Na₂SO₄. White raw powder product was obtained after the solvent was removed from the DCM solution with an IKA RV 10 digital rotavapor (IKA Works, Inc., Wilmington, NC) and was further purified with an Acquity prep-UPLC system (Waters Corporation, Milford, MA) equipped with an Acquity UPLC BEH C18 1.7 μm, 2.1×50 mm column to yield the product VH032 (1, 143 mg, 60.6% yield and 98.0% purity) as a white solid. ¹H NMR (500 MHz, DMSO-d₆) δ 8.98 (s, 1H), 8.57 (t, J=6.1 Hz, 1H), 7.95 (d, J=9.3 Hz, 1H), 7.43-7.36 (m, 4H), 5.12 (d, J=3.5 Hz, 1H), 4.54 (d, J=9.4 Hz, 1H), 4.48-4.39 (m, 2H), 4.36-4.32 (m, 1H), 4.21 (dd, J=5.4 Hz, 1H), 3.72-3.60 (m, 2H), 2.44 (s, 3H), 2.08-2.00 (m, 1H), 1.93-1.89 (m, 1H), 1.88 (s, 3H), 0.93 (s, 9H). ¹³C NMR (126 MHz, DMSO-d₆) δ 172.42, 170.14, 169.55, 151.94, 148.19, 139.99, 131.64, 130.10, 129.10, 127.88, 69.34, 60.23, 59.14, 56.86, 42.10, 38.43, 35.67, 26.83, 22.80, 16.42. ESI-TOF HRMS m/z: [M+H]⁺ Calcd for C₂₄H₃₃N₄O₄S⁺ 473.2217. Found 473.2225.

### b. (2S,4R)-1-4S)-2-(tert-butyl)-21-(5,5-difluoro-7,9-Dimethyl-5H-5Λ⁴,6Λ⁴-dipyrrolo[1,2-c:2′,1′-f][1,3,2]diazaborinin-3-yl)-4,19-dioxo-6,9,12,15-tetraoxa-3,18-diazahenicosanoyl)-4-hydroxy-N-(4-O-methylthiazol-5-yl)benzyl)pyrrolidine-2-carboxamide (5, BODIPY FL VH032)

Under room temperature, VH032-PEG4-amine hydrochloride (10, 50 mg, mmol) was added to a solution of BODIPY FL propionic acid (13, 24.20 mg, 0.083 mmol) and DIPEA (29.2 mg, 0.226 mmol) in DMSO (1 mL). EDCI (21.66 mg, 0.113 mmol) and HOBt (12.21 mg, 0.090 mmol) were then added. The reaction was stirred for 3h and then the reaction mixture was directly purified by a Biotage Isolera four flash chromatography system (Biotage, LLC, Charlotte, NC) with a Sfär C₁₈ flash column and gradient mobile phase (H₂O+0.1% formic acid→acetonitrile+0.1% formic acid) to yield the product BODIPY FL VH032 (5, 45.4 mg, 64.3% yield and 95.9% purity) as a brown-red solid. ¹H NMR (500 MHz, Chloroform-d) δ 8.70 (s, 1H), 7.36 (dd, J=7.6, 4.2 Hz, 5H), 7.07 (s, 1H), 6.87 (d, J=4.0 Hz, 1H), 6.45 (s, 1H), 6.29 (d, J=4.0 Hz, 1H), 6.10 (s, 1H), 4.73 (t, J=8.0 Hz, 1H), 4.59-4.47 (m, 3H), 4.35 (dd, J=15.0, 5.4 Hz, 1H), 4.06 (d, J=11.0 Hz, 1H), 4.01 (d, J=2.7 Hz, 2H), 3.71-3.54 (m, 13H), 3.50 (t, J=5.2 Hz, 2H), 3.39 (dq, J=8.3, 5.2, 4.6 Hz, 2H), 3.28 (t, J=7.6 Hz, 2H), 2.62 (t, J=7.6 Hz, 2H), 2.56-2.47 (m, 7H), 2.24 (s, 3H), 2.18-2.10 (m, 1H), 0.94 (s, 9H). ¹³C NMR (126 MHz, DMSO-d₆) δ 170.11, 169.24, 167.47, 166.93, 157.44, 156.20, 149.80, 146.09, 142.39, 137.79, 132.76, 131.31, 129.48, 128.03, 127.26, 127.22, 127.03, 126.48, 125.80, 123.68, 118.59, 114.95, 68.78, 68.18, 68.13, 68.06, 67.94, 67.92, 67.89, 67.45, 67.22, 57.08, 54.92, 54.03, 40.01, 36.95, 36.26, 34.06, 31.97, 24.51, 22.31, 14.26, 12.84, 9.34. ESI-TOF HRMS m/z: [M+H]⁺ Calcd for C₄₆H₆₃BF₂N₇O₉S⁺ 938.4464. Found 938.4484.

2. Biology Methods

Tb-anti-GST antibody, 1,4-dithiothreitol (DTT, 1 M), Tris (1 M, pH 7.5) and DMSO were purchased from Fisher Scientific (Pittsburgh, PA). HEPES (1 M, pH 7.4) was purchased from Teknova Inc (Hollister, CA). Triton X-100, Tween-20 and bovine serum albumin (BSA) were purchased from Sigma-Aldrich (St. Louis, MO). VH-298, VH032-amine, Me-VH032-amine and VH032-PEG4-NH₂ were purchased from MedChemExpress LLC (Monmouth Junction, NJ). BOC-VH032 was purchased from LabNetwork (Cambridge, MA). VH032 phenol and thalidomide-4′-O-acetamido-alkylC4-amine were purchased from Bio-Techne Corporation (Minneapolis, MN). dBET1, (+)-JQ1, and MZ1 were purchased from Cayman Chemical (Ann Arbor, Michigan). Echo 384-well low dead volume (LDV) compound plates were purchased from Labcyte Inc. (San Jose, CA). 384-well, black low-volume assay plates were purchased from Corning Incorporated Life Sciences (Tewksbury, MA).

c. GST-VCB Protein Preparation

The pGEX-4T-1-GST-VHL (54-213 aa) plasmid, pCDFDuet-1-flag-Elongin-C (17-112 aa)-strep II-Elongin-B (1-118 aa) plasmid and the GST-VCB protein complex were custom created, expressed and purified by GenScript USA, Inc. (Piscataway, NJ). Briefly, VHL (54-213 aa) was subcloned into the pGEX-4T-1-GST bacterial expression vector between the BamHI and XhoI restriction sites. Flag-Elongin-C (17-112 aa) and strep II-Elongin-B (1-118 aa) were respectively subcloned between the NcoI and HindIII restriction sites and between the NdeI and XhoI restriction sites into the pCDFDuet-1 bacterial expression vector. E. coli BL21(DE3) competent cells were transformed with the recombinant pGEX-4T-1-GST-VHL (54-213 aa) and pCDFDuet-1-flag-Elongin-C (17-112 aa)-strep II-Elongin-B (1-118 aa) plasmids. A single colony was inoculated into LB medium containing ampicillin and streptomycin and the culture was incubated in 37° C. at 200 rpm. Once the cell density reached to OD=0.6-0.8 at 600 nm, 0.5 mM IPTG was introduced for induction at 25° C. for 16 h. Cells were then harvested and lysed. The supernatant of the cell lysate was subjected to a one-step purification by a GST column to provide the GST-VCB protein complex. Aliquots of the GST-VCB protein was stored under −80° C. in the buffer of 50 mM Tris (pH 8.0), 150 mM NaCl and 10% Glycerol.

The protein sequence of the N-terminal GST-VHL (54-213 aa) protein is as follows: GST-Thrombin cleavage site-TEV cleavage site-VHL (54-213 aa, NCBI Reference Sequence: NP 000542.1).

(SEQ ID NO: 1)
MSPILGYWKIKGLVQPTRLLLEYLEEKYEEHLYERDEGDKWRNKKFELG
LEFPNLPYYIDGDVKLTQSMAIIRYIADKHNMLGGCPKERAEISMLEGA
VLDIRYGVSRIAYSKDFETLKVDFLSKLPEMLKMFEDRLCHKTYLNGDH
VTHPDFMLYDALDVVLYMDPMCLDAFPKLVCFKKRIEAIPQIDKYLKSS
KYIAWPLQGWQATFGGGDHPPKSDLVPRGSENLYFQGMEAGRPRPVLRS
VNSREPSQVIFCNRSPRVVLPVWLNFDGEPQPYPTLPPGTGRRIHSYRG
HLWLFRDAGTHDGLLVNQTELFVPSLNVDGQPIFANITLPVYTLKERCL
QVVRSLVKPENYRRLDIVRSLYEDLEDHPNVQKDLERLTQERIAHQRMG
D.

The protein sequence of the N-terminal Flag-Elongin-C (17-112 aa) is as follows: Flag-Elongin-C (17-112 aa, NCBI Reference Sequence: XP_028374114.1).

(SEQ ID NO: 2)
MDYKDDDDKYVKLISSDGHEFIVKREHALTSGTIKAMLSGPGQFAENET
NEVNFREIPSHVLSKVCMYFTYKVRYTNSSTEIPEFPIAPEIALELLMA
ANFLDC.

The protein sequence of the N-terminal Strep II-Elongin-B (1-118 aa) is as follows: Strep II-Elongin-B (1-118 aa, NCBI Reference Sequence: NP_009039.1).

(SEQ ID NO: 3)
MWSHPQFEKGGGSGGGSGGGSWSHPQFEKDVFLMIRRHKTTIFTDAKES
STVFELKRIVEGILKRPPDEQRLYKDDQLLDDGKTLGECGFTSQTARPQ
APATVGLAFRADDTFEALCIEPFSSPPELPDVMKPQDSGSSANEQAVQ.

d. General TR-FRET and FP Binding Assay Protocol

The final DMSO concentration was 0.2% in all tests with 0.1% DMSO from the stock probe BODIPY FL VH032 (5) solution (1000×DMSO stock) and 0.1% DMSO from test compound stock solution (1000×DMSO stock). If there is no chemical tested under certain conditions, 0.1% DMSO was supplemented to make final 0.2% for these assay condition. The final assay volume was 20 μL/well and all assays were performed under room temperature (˜25° C.). All assays were performed three times independently with quadruplicate sample replicates.

For the BODIPY FL VH032 (5)-mediated pVHL TR-FRET binding assay, a reported TR-FRET assay protocol (Lin, et al. (2014) Development of BODIPY FL vindoline as a novel and high-affinity pregnane X receptor fluorescent probe. Bioconjug Chem 25, 1664-1677) was followed except that an Echo 555 Acoustic Liquid Handler (Labcyte Inc., San Jose, CA) was used to dispense chemicals and BODIPY FL VH032 (5), GST-VCB and Tb-anti-GST antibody were used.

For the BODIPY FL VH032 (5)-mediated pVHL FP binding assay, the general TR-FRET assay protocol was followed except that the Tb-anti-anti-GST antibody was not added and the PHERAstar FS plate reader (BMG Labtech; Durham, NC) was equipped with a FP optic module (Excitation: 485 nm, Emission: 520 nm) to read FP assay signals.

e. pVHL TR-FRET Binding Assay Buffer

The pVHL TR-FRET binding assay buffer has a formula of 50 mM Tris pH 7.5, 0.1% Triton X-100, 0.01% bovine serum albumin, 1 mM DTT. It was freshly prepared every time before an experiment.

f. pVHL FP Assay Buffer

The pVHL FP assay buffer has a formula of 25 mM HEPES pH 7.4, 0.01% Tween-20, 0.5 mM DTT, 0.01% BSA. It was freshly prepared every time before an experiment.

g. Chemical Stock Solution Preparation and Reagent Dispense

Chemicals, including the fluorescent probe BODIPY FL VH032 (5), were solubilized in DMSO as 1,000× stock. Stock chemical DMSO solutions, positive control VH298 (2) and negative control DMSO were all plated in Echo LDV compound plates. For the probe or chemicals tested in dilutions, their stock dilutions were prepared in Echo LDV compound plates as 1,000×DMSO stocks for all concentration levels. During TR-FRET and FP assays, assay buffer (10 μL/well) was first dispensed. Fluorescent probe BODIPY FL VH032 (5) 1,000× time DMSO stock in dilutions or single concentration was dispensed (20 μL/well) with an Echo 555 Acoustic Liquid Handler, and then 1,000× time DMSO stock positive control VH298 (2), negative control DMSO, chemicals or dilutions of chemicals was transferred (20 nL/well) with the Echo 555 Acoustic Liquid Handler. Protein solution (2× stock, 10 μL/well) in corresponding assay buffer was finally dispensed to give a total of 20 μL/well assay volume. The fluorescent probe, each chemical, the positive control VH298 (2) or negative control DMSO (20 nL/well) was dispensed to a final total volume of 20 μL/well to achieve the 1-to-1,000× dilution. To avoid possible signal variation introduced by different DMSO concentrations (Lin, W., and Chen, T. (2018) Using TR-FRET to Investigate Protein-Protein Interactions: A Case Study of PXR-Coregulator Interaction. Adv Protein Chem Struct Biol 110, 31-63; Lin, W., Liu, J., Jeffries, C., Yang, L., Lu, Y., Lee, R. E., and Chen, T. (2014) Development of BODIPY FL vindoline as a novel and high-affinity pregnane X receptor fluorescent probe. Bioconjug Chem 25, 1664-1677; Lin, W., and Chen, T. (2013) A vinblastine fluorescent probe for pregnane X receptor in a time-resolved fluorescence resonance energy transfer assay. Anal Biochem 443, 252-260), the 0.2% DMSO concentration was used for all assays in this article.

h. Determination of BODIPY FL VH032 (5) Binding K_{D} to GST-VCB Protein Complex in a TR-FRET Binding Assay

Dilutions of BODIPY FL VH032 (5, 1-to-2 dilutions, a concentration range of 0.06 nM to 500 nM) was incubated with 2 nM Tb-anti-GST along with, group 1: 2 nM GST-VCB+DMSO; group 2: 2 nM GST-VCB+VH298 (2, 30 μM); or group 3: without GST-VCB+DMSO. The TR-FRET signals were monitored every 30 min from 30 min to 300 min with a PHERAstar FS plate reader equipped with a TR-FRET optic module (excitation: 340 nm; emission 1: 520 nm; emission 2: 490 nm). The TR-FRET signals were fitted into GraphPad Prism 8.4.3 software (GraphPad Software; San Diego, CA) using a one-site total binding equation to derive curves for each group. The binding affinity K_{d} values were derived from the group 1 with 2 nM GST-VCB.

1. Signal Stability Test of the BODIPY FL VH032 (5)-Mediated PVHL TR-FRET Binding Assay

BODIPY FL VH032 (5, 4 nM) and 2 nM Tb-anti-GST was incubated with, group 1 (negative control group): 2 nM GST-VCB+DMSO; group 2 (positive control group): 2 nM GST-VCB+VH298 (2, 30 μM); group 3 (background control group): without GST-VCB+DMSO; or group 4 (positive control dose response group): 2 nM GST-VCB+dilutions of VH298 (2, 1-to-3 dilutions, a concentration range of 2.1 pM to 30 μM). The TR-FRET signals were monitored every 30 min from 30 min to 300 min. The TR-FRET signal of group 1, 2 or 3 was divided by that of group 2 to derive the TR-FRET signal fold change of each group. The TR-FRET signals or the signal fold changes of the group 1, 2 and 3 were plotted. The dose dependent TR-FRET signals of the positive control VH298 (2, group 4) were fitted into the GraphPad Prism software using a Sigmoidal dose-response equation to derive IC₅₀ values.

Binding inhibitory activity test of selected pVHL ligands or non-ligands with the BODIPY FL VH032 (5)-mediated pVHL TR-FRET binding assay. BODIPY FL VH032 (5, 4 nM) was incubated with the positive control VH298 (2, 30 μM), negative control DMSO, or dilutions of selected pVHL ligands or non-ligands (1-to-3 dilutions, a concentration range of 2.1 pM to 30 μM), along with 2 nM GST-VCB and 2 nM Tb-anti-GST. The TR-FRET signals were determined at the 90-min incubation time. The % Inhibition of each tested ligand at its individual concentration was calculated by normalized to that of the positive control VH298 (2, 30 μM) and negative control DMSO using Equation 1.

$$\% inhibition = 100 \% - 100 \% \times \frac{( {Signal}_{Ligand} - {Signal}_{30 μ M VH 298} )}{( {Signal}_{DMSO} - {Signal}_{30 μ M VH 298} )} \qquad Equation 1$$

When applicable, the normalized percent inhibition values for each ligand at various concentrations were fitted into a sigmoidal dose-response equation with the GraphPad Prism software to derive the IC₅₀ values. The TR-FRET Kᵢ values were then calculated with Equation 2 (the Cheng-Prusoff equation) (Cheng, Y., and Prusoff, W. H. (1973) Relationship between the inhibition constant (K1) and the concentration of inhibitor which causes 50 percent inhibition (I50) of an enzymatic reaction. Biochem Pharmacol 22, 3099-3108).

Kᵢ=IC₅₀/(1+[L]/K_{L}) Equation 2

Where IC₅₀ is the concentration of tested ligand that inhibits 50% of BODIPY FL VH032 (5) binding to GST-VCB, [L] is the BODIPY FL VH032 (5) concentration of 4 nM in the assay mixture, and K_{L} is the K_{d} value of BODIPY FL VH032 (5) in the assay which is 3.01 nM. The TR-FRET Kᵢ values were used to compare the relative binding affinities of the test ligands to pVHL.

j. BODIPY FL VH032 (5) Concentration Optimization in a pVHL FP Assay

BODIPY FL VH032 (5, 70, 60, 50, 40, 30, 20, 10, 5, 2, or 1 nM) was incubated with dilutions of GST-VCB (1-to-2 dilutions, a concentration range of 0.03 nM to 1 μM). The FP signals were monitored with the PHERAstar FS plate reader equipped with a FP optic module (Excitation: 485 nm, Emission: 520 nm). The representative data collected at 90-min incubation time was plotted with the GraphPad Prism software.

k. Determination of BODIPY FL VH032 (5) Binding K_{D} to GST-VCB Protein Complex in a FP Binding Assay

BODIPY FL VH032 (5, 10 nM) was incubated with dilutions of GST-VCB (1-to-2 dilutions, a concentration range of 0.03 nM to 1 μM) along with the DMSO group or VH298 (2, 30 μM) group. In addition, BODIPY FL VH032 (5, 10 nM) was incubated with DMSO only, but without GST-VCB. The FP signals were determined with the PHERAstar FS plate reader. The representative data collected at 90-min incubation time was plotted with the GraphPad Prism software using a one-site total binding equation to derive curves for each group. The binding affinity K_{d} values were derived from the DMSO with GST-VCB.

l. Binding Activity Test of Selected pVHL Ligands or Non-Ligands with the Bodipy FL VH032 (5)-Mediated pVHL FP Binding Assay

BODIPY FL VH032 (5, 10 nM) was incubated with the positive control VH298 (2, 30 μM), negative control DMSO, or dilutions of selected pVHL ligands or non-ligands (1-to-3 dilutions, a concentration range of 2.1 pM to 30 μM), along with 100 nM GST-VCB. The FP signals were determined at the 90-min incubation time. The FP signal fold change of the negative control DMSO group or the positive control VH298 (2, 30 μM) group was divided by that of the positive control VH298 (2, 30 μM) group to derive FP signal fold changes. The FP signals or the FP signal fold changes were plotted with the GraphPad Prism software. The % Inhibition of each tested ligand at its individual concentration was calculated by normalized to that of the positive control VH298 (2, 30 μM) and negative control DMSO using Equation 1. The FP Kᵢ values were calculated by the method developed by Nikolovska-Coleska et al. ((2004) Development and optimization of a binding assay for the XIAP BIR3 domain using fluorescence polarization. Anal Biochem 332, 261-273) with the Kᵢ calculator available at: http://www.umich.edu/˜shaomengwanglab/software/calc_ki/index.html. The FP Kᵢ values were used to compare the relative binding affinities of the test ligands to pVHL.

3. Results and Discussion

a. Synthesis of VH032 (1)

VH032 (1) is a potent VHL inhibitor (Frost, et al. (2016) Potent and selective chemical probe of hypoxic signalling downstream of HIF-α hydroxylation via VHL inhibition. Nat Commun 7, 13312; Soares, et al. (2018) Group-Based Optimization of Potent and Cell-Active Inhibitors of the von Hippel-Lindau (VHL) E3 Ubiquitin Ligase: Structure-Activity Relationships Leading to the Chemical Probe (2S,4R)-1-((S)-2-(1-Cyanocyclopropanecarboxamido)-3,3-dimethyl butanoyl)-4-hydroxy-N-(4-(4-methylthiazol-5-yl)benzyl)pyrrolidine-2-carboxamide (VH298). J Med Chem 61, 599-618) and suitable for fluorescent probe development as a positive ligand control. However, it is not commercially available. VH032 (1) was prepared by acetylating the VH032 amine (6) with acetic anhydride (Ac₂O) in the presence of N,N-diisopropylethylamine (DIPEA) with dichloromethane (DCM) as the solvent. The yield is 60.6% after purification with a Prep-HPLC system (Scheme 1).

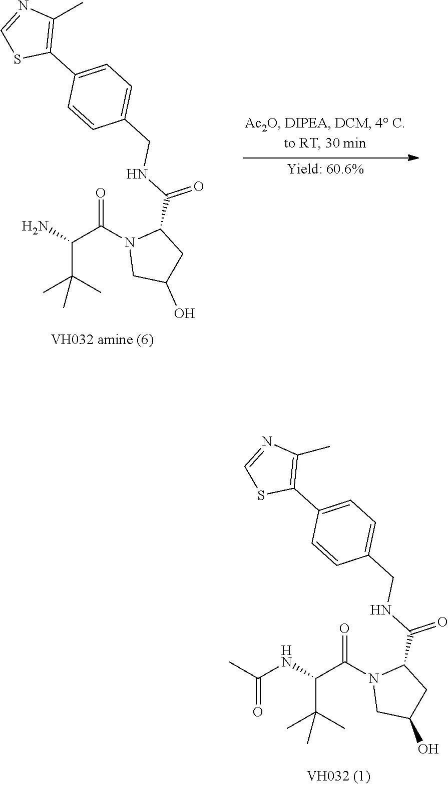 [SMILES: Cc1ncsc1-c1ccc(CNC(=O)[C@@H]2CC(O)CN2C(=O)[C@@H](N)C(C)(C)C)cc1 ; CC(=O)N[C@H](C(=O)N1C[C@H](O)C[C@H]1C(=O)NCc1ccc(-c2scnc2C)cc1)C(C)(C)C]

b. Design of BODIPY FL VH032 (5)

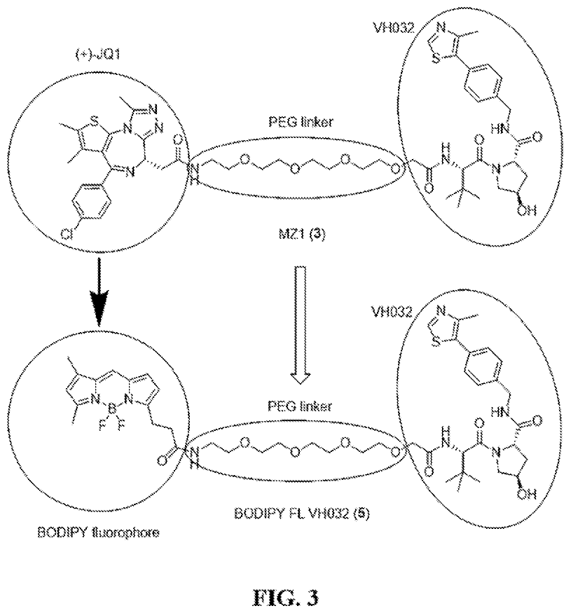

The BODIPY FL VH032 (5) was designed based on the MZ1 (3). MZ1 (3) is a bivalent BRD-VHL PROTAC molecule with the BRD ligand (+)-JQ1 (4) joined to the VHL ligand VH032 (1) by a PEG linker (Gadd, et al. (2017) Structural basis of PROTAC cooperative recognition for selective protein degradation. Nat Chem Biol 13, 514-521). It maintains binding affinities to both BRD and pVHL proteins via its corresponding (+)-JQ1 (4) moiety and its VH032 (1) moiety. It was rationalized that a high affinity pVHL fluorescent probe will be obtained if the (+)-JQ1 (4) moiety in MZ1 (3) is replaced with a fluorescent moiety, such as a BODIPY fluorophore, while the PEG linker and VH032 portions remain intact (FIG. 1). The BODIPY FL VH032 (5) was, thus, designed accordingly (FIG. 3).

c. Synthesis of BODIPY FL VH032 (5)

The BODIPY FL VH032 (5) was prepared with an EDCI- and HOBt-mediated coupling (Chan, L. C., and Cox, B. G. (2007) Kinetics of Amide Formation through Carbodiimide/N-Hydroxybenzotriazole (HOBt) Couplings. The Journal of Organic Chemistry 72, 8863-8869) between VH032-PEG4-amine (10) and BODIPY FL propionic acid (13) in the presence of DIPEA under room temperature with a yield of 64.3% after flash column chromatography purification (Scheme 2).

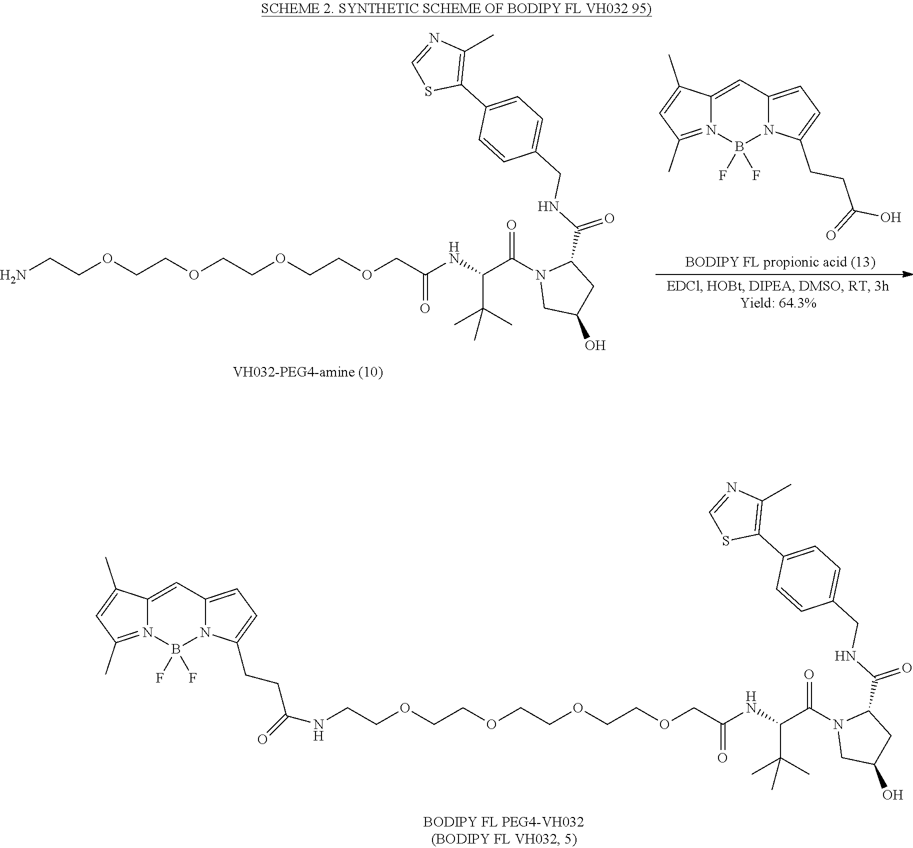

d. BODIPY FL VH032 (5) Displayed a High Binding Affinity to pVHL in a TR-FRET Assay

To measure the binding affinity of BODIPY FL VH032 (5) to pVHL, dilutions of BODIPY FL VH032 (5, 1-to-2 dilutions, an optimized concentration range of 0.06 nM to 500 nM) were incubated with 2 nM Tb-anti-GST antibody in the presence of 2 nM GST-VCB. By comparison, groups of samples without GST-VCB or with additional VH298 (2, 30 μM) in the presence of 2 nM GST-VCB were also included to investigate the background interactions between the BODIPY FL VH032 (5) and the Tb-anti-GST antibody or between the BODIPY FL VH032 (5) and the complex of Tb-anti-GST antibody and GST-VCB protein in the presence of VH298 (2). The TR-FRET signals were measured at every 30 min from 30 min to 300 min.

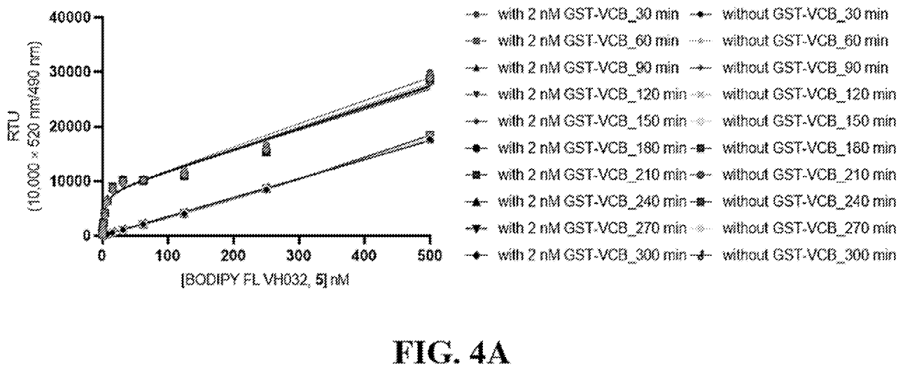

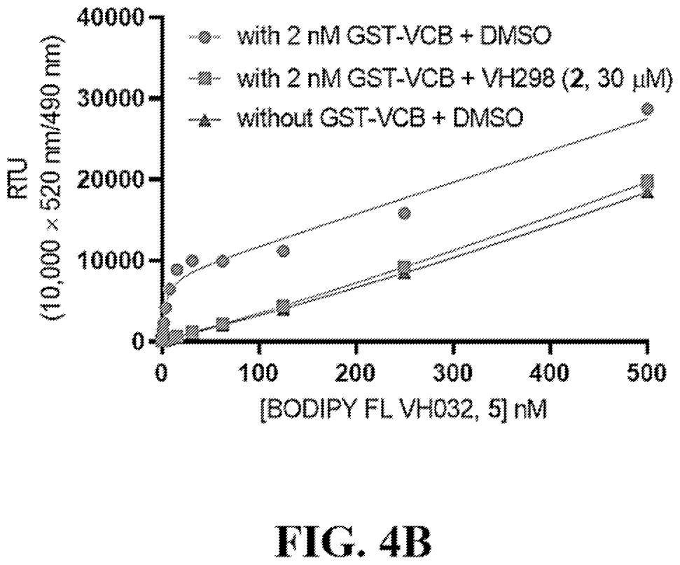

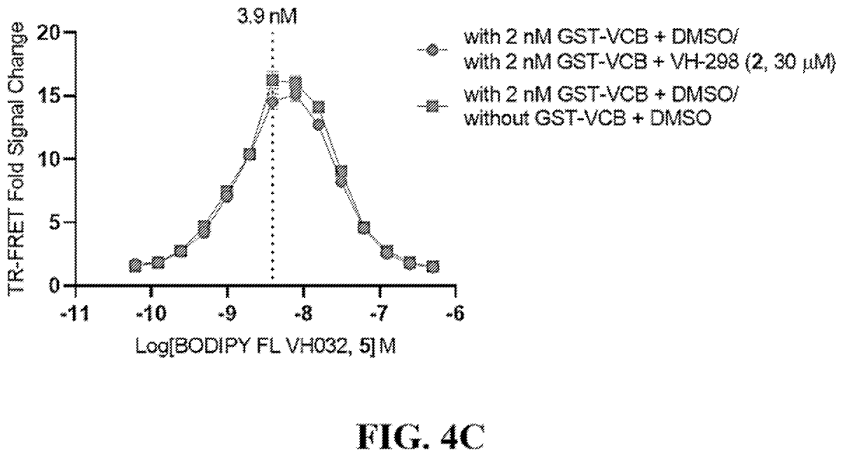

The TR-FRET signals of the groups with 2 nM GST-VCB and without GST-VCB were first analyzed by fitting in a one-site total binding equation with the GraphPad PRISM software (GraphPad Software; San Diego, CA) (FIG. 4A). In the presence of Tb-anti-GST, the interaction between BODIPY FL VH032 (5) and GST-VCB increased exponentially in the BODIPY FL VH032 (5) concentration range of 0.06 nM to 15.6 nM, and then the interaction increased in a linear manner in the BODIPY FL VH032 (5) concentration range of 15.6 nM to 500 nM (top panel curves in FIG. 4A). The binding dissociation constant (K_{d}) values were derived from the 2 nM GST-VCB group and the respective K_{d} values were 3.61, 3.22, 3.01, 3.04, 3.01, 2.98, 2.96, 2.98, 2.99 and 3.04 nM for the incubation times of 30, 90, 120, 150, 180, 210, 240, 270, and 300 min. The binding dissociation constant (K_{d}) values were basically very stable at ca 3.0 nM from the 90 min to 300 min incubation time. In addition, the K_{d} value of ca 3.0 nM demonstrated it is a high affinity interaction between BODIPY FL VH032 (5) and GST-VCB. Foremost, BODIPY FL VH032 (5) is so far reported the first small molecule-based pVHL fluorescent probe. In the absence of GST-VCB, a linear and very low TR-FRET interaction was observed between BODIPY FL VH032 (5) and Tb-anti-GST in the entire BODIPY FL VH032 (5) concentration range of 0.06 nM to 500 nM which represents a low non-specific background interaction nature (bottom panel curves in FIG. 4A) (Lin, W., and Chen, T. (2018) Using TR-FRET to Investigate Protein-Protein Interactions: A Case Study of PXR-Coregulator Interaction. Adv Protein Chem Struct Biol 110, 31-63; Lin, W., Liu, J., Jeffries, C., Yang, L., Lu, Y., Lee, R. E., and Chen, T. (2014) Development of BODIPY FL vindoline as a novel and high-affinity pregnane X receptor fluorescent probe. Bioconjug Chem 25, 1664-1677).

Because the 90 min incubation time was the earliest signal stable time point, the 90 min time point was chosen for further examination with an additional test group, the group with 2 nM GST-VCB+VH298 (2, 30 μM), included. The three groups of data, 2 nM GST-VCB+DMSO, 2 nM GST-VCB+VH298 (2, 30 μM) and without GST-VCB+DMSO, at the 90 min incubation time point were graphed by fitting into the one-site total binding equation in GraphPad PRISM (FIG. 4B). The curve derived from the group of data with 2 nM GST-VCB+DMSO represented the total interaction between BODIPY FL VH032 (5) and GST-VCB in the presence of Tb-anti-GST with a K_{d} value of 3.01 nM (the top blue curve in FIG. 4B). In the presence of VH298 (2, 30 μM) which is a potent pVHL inhibitor (Frost, J, et al. (2016) Potent and selective chemical probe of hypoxic signalling downstream of HIF-α hydroxylation via VHL inhibition. Nat Commun 7, 13312; Soares, P., et al. (2018) Group-Based Optimization of Potent and Cell-Active Inhibitors of the von Hippel-Lindau (VHL) E3 Ubiquitin Ligase: Structure-Activity Relationships Leading to the Chemical Probe (2S,4R)-1-4S)-2-(1-Cyanocyclopropanecarboxamido)-3,3-dimethylbutanoyl)-4-hydroxy-N-(4-(4-methylthiazol-5-yl)benzyl)pyrrolidine-2-carboxamide (VH298). J Med Chem 61, 599-618), as the red curve in the FIG. 4B, the TR-FRET signal between BODIPY FL VH032 (5) and GST-VCB in the presence of Tb-anti-GST is very close to the TR-FRET signal observed in the group without GST-VCB (the background interaction curve or the green curve in FIG. 4B). The overlap of VH298 (2, 30 μM) inhibited curve with the background curve demonstrated there was minimal non-specific interaction between BODIPY FL VH032 (5) and GST-VCB.

In order to develop a TR-FRET assay to characterize pVHL ligands for their inhibitory binding activities, the concentration of the fluorescent probe BODIPY FL VH032 (5) is very important. To gain the insight of the most sensitive BODIPY FL VH032 (5) concentration for a TR-FRET assay, the fold changes of TR-FRET signal were plotted at various BODIPY FL VH032 (5) concentrations between two different group comparisons: with 2 nM GST-VCB+DMSO/with 2 nM GST-VCB+VH-298 (2, 30 μM) (blue curve in FIG. 4C) and with 2 nM GST-VCB+DMSO/without GST-VCB+DMSO (red curve in FIG. 4C). The curves of signal fold change overlapped well with only very small difference. The highest signal fold changes were observed at 3.9 or 7.8 nM of BODIPY FL VH032 (5): with 2 nM GST-VCB+DMSO/with 2 nM GST-VCB+VH-298 (2, 30 pM) curve at 7.8 nM (15.1-fold) and 2 nM GST-VCB+DMSO/without GST-VCB+DMSO curve at 3.9 nM (16.2-fold). Because using a probe concentration closer to its K_{d} concentration in an assay will lead to less deviation of Kᵢ calculation of tested ligands (Cheng, Y., and Prusoff, W. H. (1973) Relationship between the inhibition constant (K1) and the concentration of inhibitor which causes 50 percent inhibition (I50) of an enzymatic reaction. Biochem Pharmacol 22, 3099-3108), the BODIPY FL VH032 (5) at 4 nM which is close to its K_{d} concentration of 3.01 nM was selected for the further TR-FRET assay development.

Referring to FIG. 4A, the binding interaction of BODIPY FL VH032 (5) to 2 nM Tb-anti-GST in the presence of 2 nM GST-VCB or in the absence of GST-VCB at the designated incubation time is shown. The TR-FRET signals were expressed as relative TR-FRET units (RTU) which were calculated by 10,000×520 nm/490 nm.

Referring to FIG. 4B, the binding interaction of BODIPY FL VH032 (5) to 2 nM Tb-anti-GST, with 2 nM GST-VCB, 2 nM GST-VCB+VH298 (2, 30 μM), or without GST-VCB+DMSO at the 90-min incubation time is shown.

Referring to FIG. 4C, fold changes of TR-FRET signal of BODIPY FL VH032 (5) with 2 nM GST-VCB+DMSO to 2 nM GST-VCB+VH298 (2, 30 μM) (blue curve), or to without GST-VCB+DMSO (red curve) are shown.

e. The BODIPY FL VH032 (5)-Based pVHL TR-FRET Binding Assay has Stable Signal

An assay with stable signal is very important for obtaining consistent activities of tested compounds. The BODIPY FL VH032 (5)-mediated pVHL TR-FRET binding assay has been established using the following assay conditions: 4 nM BODIPY FL VH032 (5), 2 nM GST-VCB, 2 nM Tb-anti-GST and a tentative 90-min incubation time based on the signal stability observed without competing compound

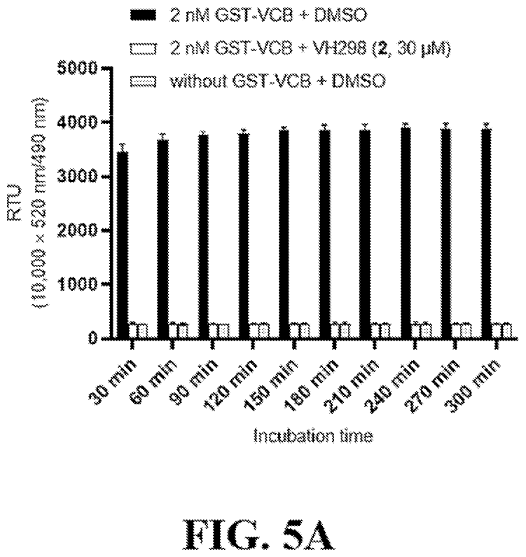

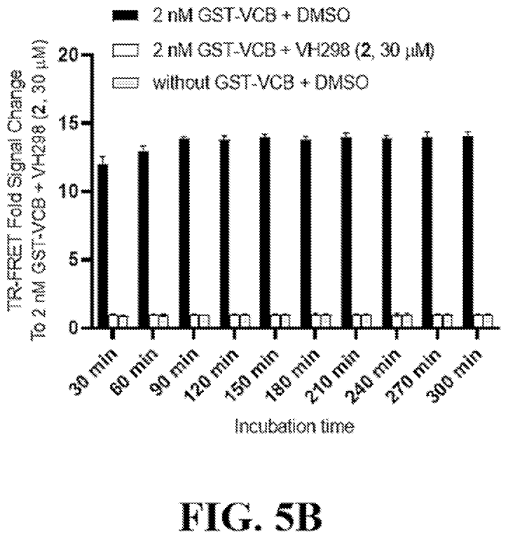

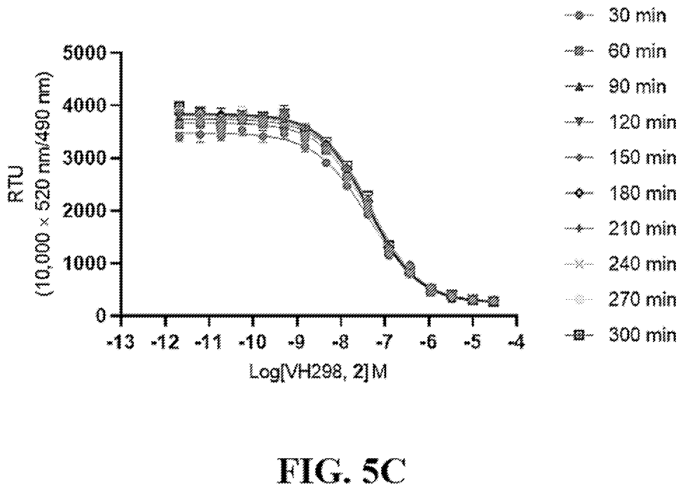

To further evaluate the assay signal stability in the presence of competing compound, a potent pVHL inhibitor VH298 (2, 30 μM) was used as the positive control, and DMSO was used as the negative control (DMSO is a solvent used to prepare compound stock solutions). In addition, the group of samples with 4 nM BODIPY FL VH032 (5), 2 nM Tb-anti-GST and DMSO, but without GST-VCB was included as the background control. The performance of the controls at different incubation time points was summarized in the form of relative TR-FRET unit (FIG. 5A) and signal fold change to 2 nM GST-VCB+VH298 (2, μM) (FIG. 5B). Consistent to the observation made without competing compound, the positive control group had TR-FRET signals (278.7±4.5) or the signal fold change to 2 nM GST-VCB+VH298 (2, 30 μM) (1.00±0.03) that were very similar to those (276.6±5.4 for TR-FRET signals and 0.99±0.03 for the fold signal change) of the background control group at all incubation times from 30 min to 300 min (FIG. 5B). For the negative control group, the overall TR-FRET interaction signals was slightly low at 30 min and 60 min incubation time points with respective RTUs at 3451 count and 3679 count, but very stable with RTU at 3858±46 from incubation times of 90 min to 300 min (FIG. 5A). In terms of signal fold change to 2 nM GST-VCB+VH298 (2, 30 μM) (FIG. 5B), the negative control group had 12.05 and 12.94-fold at corresponding 30- and 60-min incubation times, then stable at 13.93±0.09 with a CV of 0.67% from the incubation time of 90 min to 300 min, which is consistent to the observation made without competing compound. Thus, the BODIPY FL VH032 (5)-mediated pVHL TR-FRET binding assay maintains stable signal from incubation time of 90 min to 300 min.

The signal stability of the assay was further evaluated with the positive control VH298 (2) in a dose dependent manner (1-to-3 dilutions, an optimized concentration range of 2.09 pM to 30 μM) (FIG. 5C). IC₅₀ values of 42.17, 43.27, 44.46, 42.93, 43.20, 44.04, 43.86, 45.13, and 48.27 nM were observed at respective time point from 30 min to 300 min, with the average IC₅₀ value being 44.31 nM with a standard deviation of 1.75 nM and a low CV of 3.95%. These results further demonstrated that the BODIPY FL VH032 (5)-mediated pVHL TR-FRET binding assay has stable signal over a wide range of incubation times. 90-min was chosen as the incubation time for the assay.

Referring to FIG. 5A, TR-FRET interaction of 4 nM BODIPY FL VH032 (5) and 2 nM Tb-anti-GST with 2 nM GST-VCB+DMSO (negative control), 2 nM GST-VCB+VH298 (2, 30 μM) (positive control), or without GST-VCB+DMSO (background control) at specified incubation time points is shown.

Referring to FIG. 5B, TR-FRET signal fold change to 2 nM GST-VCB+VH298 (2, 30 μM) of 2 nM GST-VCB+DMSO (negative control), 2 nM GST-VCB+VH298 (2, 30 μM) (positive control) or without GST-VCB+DMSO (background control) in the presence of 4 nM BODIPY FL VH032 (5) and 2 nM Tb-anti-GST at specified incubation time points is shown.

Referring to FIG. 5C, dose response inhibition curves of VH298 (2, 1-to-3 dilutions, a concentration range of 2.1 pM to 30 μM) at specified incubation time points in the presence of 4 nM BODIPY FL VH032 (5), 2 nM GST-VCB, and 2 nM Tb-anti-GST is shown.

f. The BODIPY FL VH032 (5)-Mediated pVHL TR-FRET Binding Assay is Sensitive and Selective

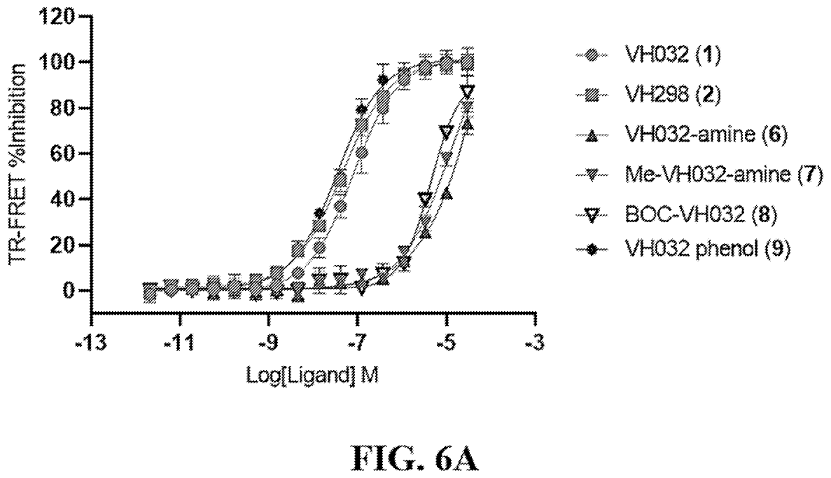

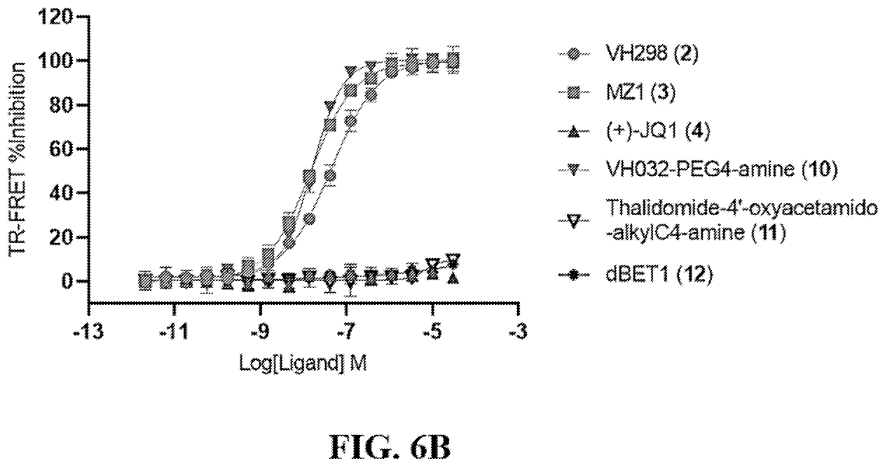

A panel of reported pVHL ligands including VH032 (1), VH298 (2), VH032 amine (6), Me-VH032 amine (7), BOC-VH032 (8), VH032 phenol (9), VH032-PEG4-amine (10), dual pVHL and BRD PROTAC ligand MZ1 (3), and non-pVHL ligands including (+)-JQ1 (4), Thalidomide-4′-oxyacetamido-alkylC4-amine (11) and dBET1 (12) were tested for their pVHL inhibitory activities in the new established BODIPY FL VH032 (5)-mediated pVHL TR-FRET binding assay with an optimized assay condition of 4 nM BODIPY FL VH032 (5), 2 nM GST-VCB, 2 nM Tb-anti-GST and a 90-min incubation time, along with negative control DMSO and positive control VH298 (2, 30 μM). The dose response curves of the pVHL ligands VH032 (1), VH298 (2), VH032 amine (6), Me-VH032 amine (7), BOC-VH032 (8), and VH032 phenol (9) are summarized in FIG. 6A. The dose response curves of PROTACs MZ1 (3) and dBET1 (12), together with the PROTAC components (+)-JQ1 (4), VH032-PEG4-amine (10) and Thalidomide-4′-oxyacetamido-alkylC4-amine (11), as well as the positive control VH298 (2) are listed in FIG. 6B. The pVHL ligands VH032 (1), VH298 (2), VH032 amine (6), Me-VH032 amine (7), BOC-VH032 (8), VH032 phenol (9), VH032-PEG4-amine (10), dual pVHL and BRD PROTAC ligand MZ1 (3) had respective IC₅₀ values of 77.8 nM, 44.0 nM, 13.3 μM, 7.9 μM, 4.9 μM, 34.0 nM, 5.9 nM, 14.7 nM and respective Kᵢ values of 33.4 nM, 18.9 nM, 5.7 μM, 3.4 μM, 2.1 μM, 14.6 nM, 6.8 nM, and 6.3 nM. Among the pVHL ligands tested, the most potent ligand was MZ1 (3) with a Kᵢ value of 6.3 nM and the least potent ligand was VH032 amine (6) with a Kᵢ value of 5.7 μM. There was over 904-fold activity difference between the most and least potent ligands, indicating that the BODIPY FL VH032 (5)-mediated pVHL TR-FRET binding assay is sensitive to characterize pVHL ligands with high, medium or low inhibitory activities. As expected, the non-pVHL ligands (+)-JQ1 (4, a BRD ligand), Thalidomide-4′-oxyacetamido-alkylC4-amine (11, a cereblon E3 ligase ligand) and dBET1 (12, a BRD-CRBN PROTAC) were inactive (FIG. 6B), demonstrating that the assay only selectively detects pVHL ligands.

Referring to FIG. 6A, dose response curves of pVHL ligands VH032 (1), VH298 (2), VH032 amine (6), Me-VH032 amine (7), BOC-VH032 (8), and VH032 phenol (9) are shown.

Referring to FIG. 6B, dose response curves of pVHL ligands of VH298 (2), MZ1 (3), VH032-PEG4-amine (10) and non-pVHL ligands (+)-JQ1 (4, a BRD ligand), Thalidomide-4′-oxyacetamido-alkylC4-amine (11, a cereblon E3 ligase ligand), and dBET1 (12, a BRD-CRBN PROTAC) are shown.

g. BODIPY FL VH032 (5) Concentration Optimization for a pVHL FP Assay

To compare the TR-FRET assay format to an FP assay format, a pVHL FP assay was also developed with BODIPY FL VH032 (5) as the fluorescent probe.

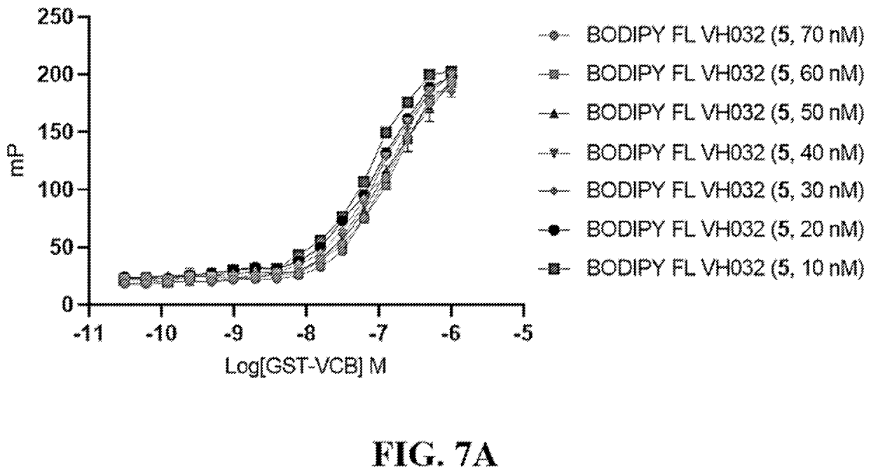

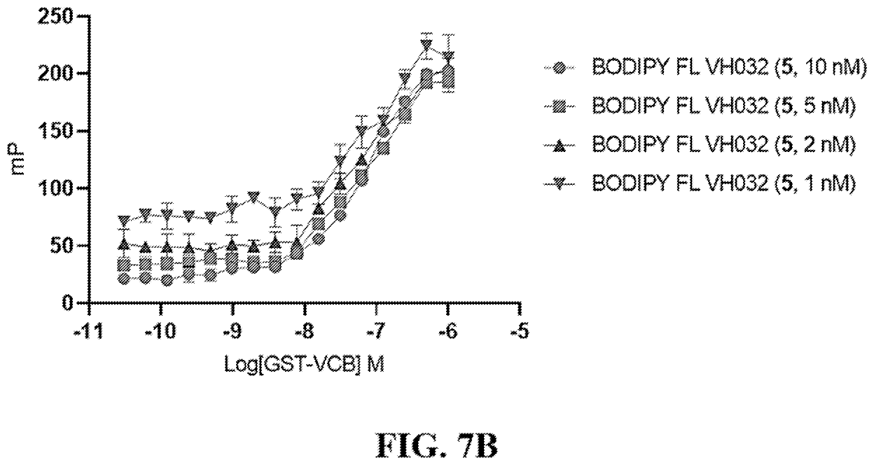

To establish an FP assay that is sensitive and robust, an optimal probe concentration is critical, because too much probe will decrease assay sensitivity while insufficient probe will reduce assay robustness. It was chosen to optimize the probe concentration by incubating dilutions of GST-VCB (1-to-2 dilutions, an optimal concentration range of 0.03 nM to 1000 nM) with BODIPY FL VH032 (5) at 70, 60, 50, 40, 20, 10, 5, 2, and 1 nM (FIG. 7A and FIG. 7B). BODIPY FL VH032 (5) at concentrations ranged from 70 nM to 10 nM (FIG. 7A) did not affect the GST-VCB concentration curves, except slight FP signal increases at certain GST-VCB concentrations (125, 250, and 500 nM) with 10 nM BODIPY FL VH032 (5). However, lower concentrations of BODIPY FL VH032 (5) (5, 2, or 1 nM) (FIG. 7B) caused a substantial background increase at lower GST-VCB concentrations (FIG. 7B), with respective background interactions increased from 21 to 33, 52 and 71 mP for BODIPY FL VH032 (5) concentrations of 10, 5, 2, and 1 nM. In addition, significant FP signal variations were observed at lower BODIPY FL VH032 (5) concentrations, especially at 2 and 1 nM (FIG. 7B). 10 nM BODIPY FL VH032 (5) was chosen for the FP assay.

Referring to FIG. 7A, pVHL FP assay performance with BODIPY FL VH032 (5) concentrations at 70, 60, 50, 40, 30, 20, and 10 nM is shown.

Referring to FIG. 7B, pVHL FP assay performance with BODIPY FL VH032 (5) concentrations at 10, 5, 2, and 1 nM is shown.

h. BODIPY FL VH032 (5) Displays High pVHL Affinity in an FP Assay

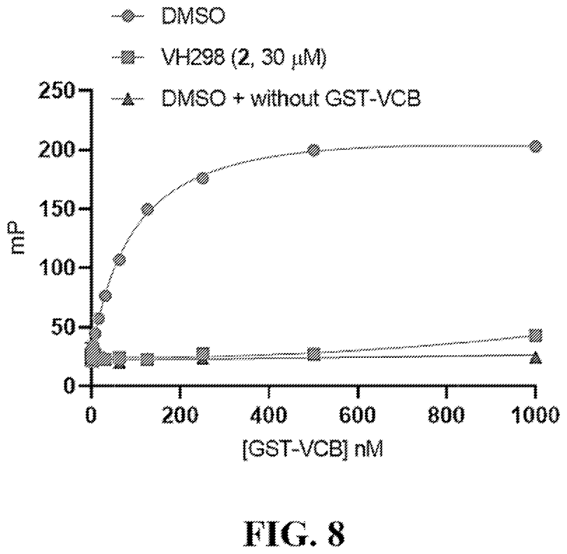

To determine the optimal GST-VCB concentration for a BODIPY FL VH032 (5)-mediated pVHL FP assay, 10 nM BODIPY FL VH032 (5) was incubated with dilutions of GST-VCB (1-to-2 dilutions, an optimized concentration range of 0.03 nM to 1000 nM) plus DMSO (total interaction) or VH298 (2, 30 μM) (GST-VCB-mediated non-specific interaction). In addition, 10 nM BODIPY FL VH032 (5) with DMSO only, but without GST-VCB was used to determine the background interaction. The FP signals from the 3 groups were fitted into the one-site total binding equation in GraphPad PRISM (FIG. 8). The curve derived from the total interaction group (DMSO group) represented the total FP interaction between BODIPY FL VH032 (5) and GST-VCB with a K_{d} value of 100.8 nM (the blue curve in FIG. 8). The FP signals from the GST-VCB-mediated non-specific interaction [in the presence of VH298 (2, 30 μM), the red curve in FIG. 8] were very similar to those of the background interaction (without GST-VCB, the green curve in FIG. 8), except that the FP signal (43.0 mP) at the highest GST-VCB concentration (1000 nM) in the presence of VH298 (2, 30 μM) was slightly higher than the background FP signal (24.8 mP). 100 nM of GST-VCB was chosen, which is 10-time lower than the 1000 nM to minimize the GST-VCB-mediated non-specific interaction (overlaps with the background signal). At 100 nM, the target protein GST-VCB has a concentration close to the K_{d} value of the fluorescent probe BODIPY FL VH032 (5) (100.8 nM), which helps maintain a balance between sensitivity and signal window for the FP assay (Nikolovska-Coleska, et al. (2004) Development and optimization of a binding assay for the XIAP BIR3 domain using fluorescence polarization. Anal Biochem 332, 261-273).

i. The BODIPY FL VH032 (5)-Mediated pVHL FP Assay is Sensitive and Selective in Detecting Ligands of pVHL

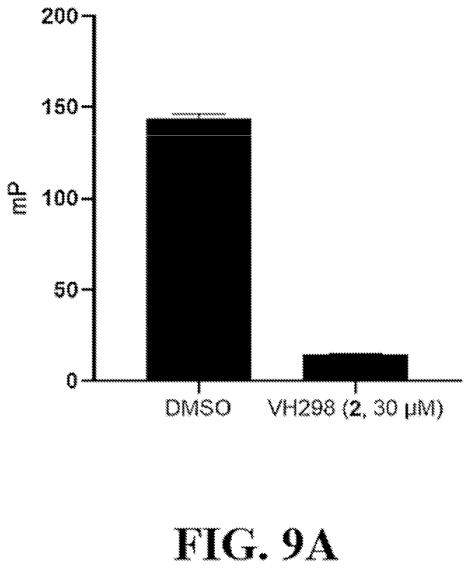

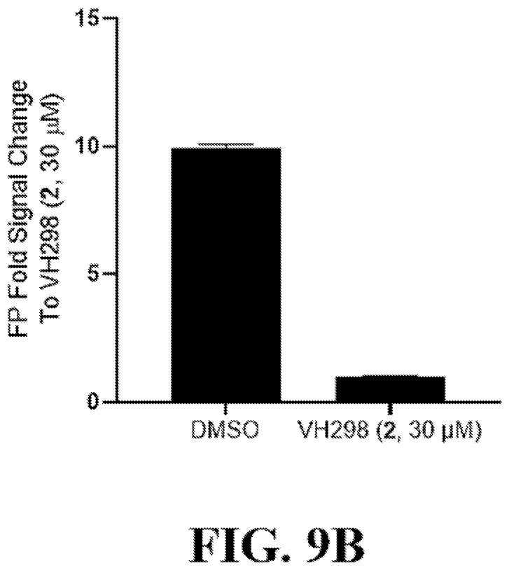

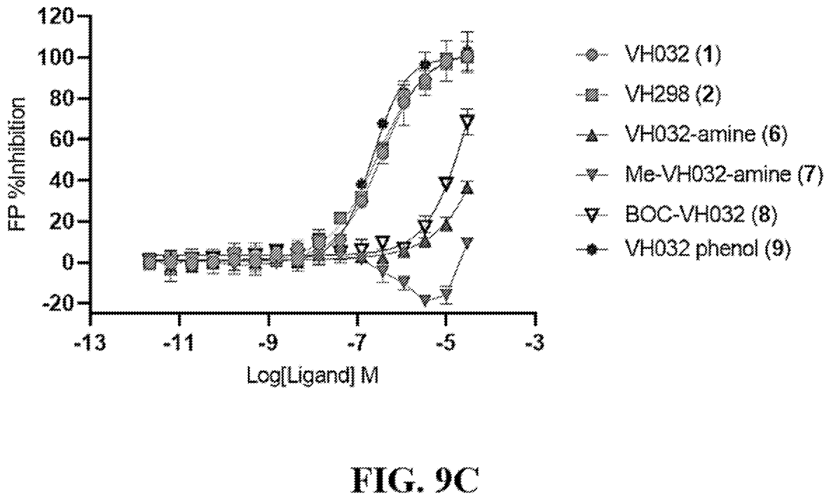

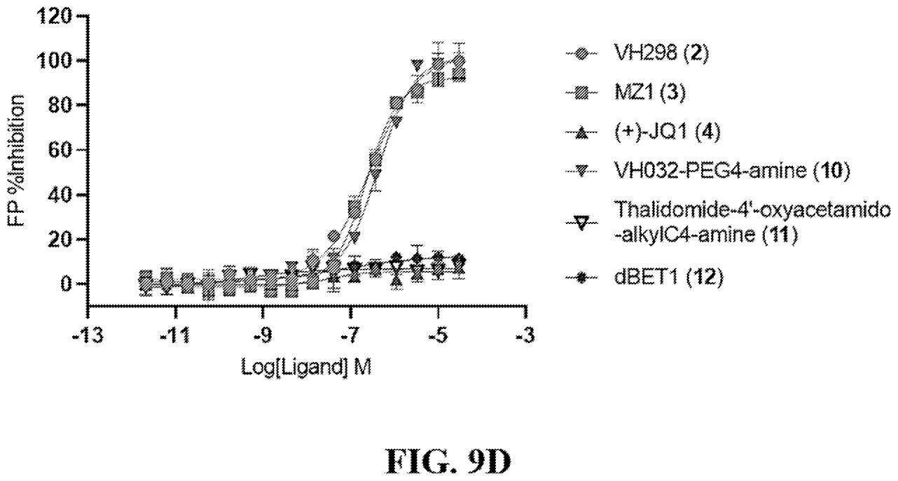

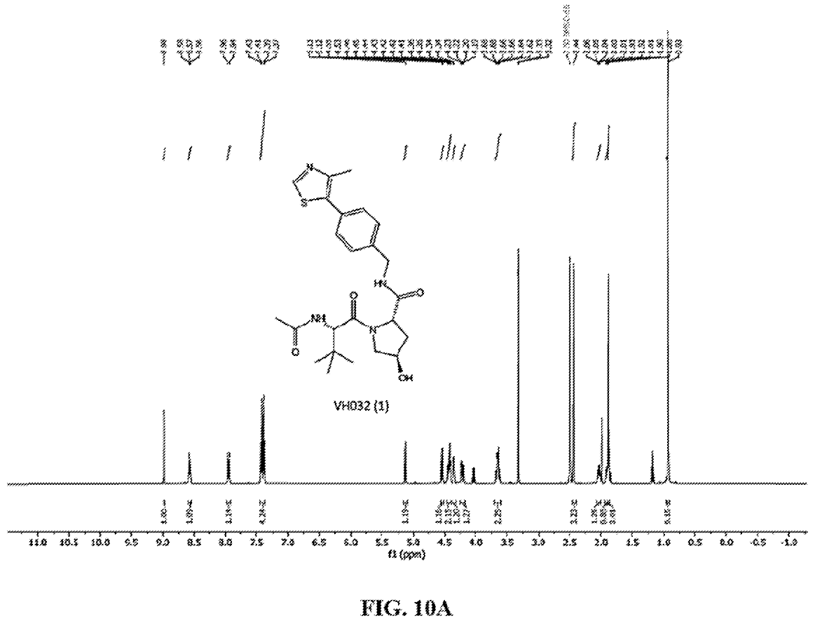

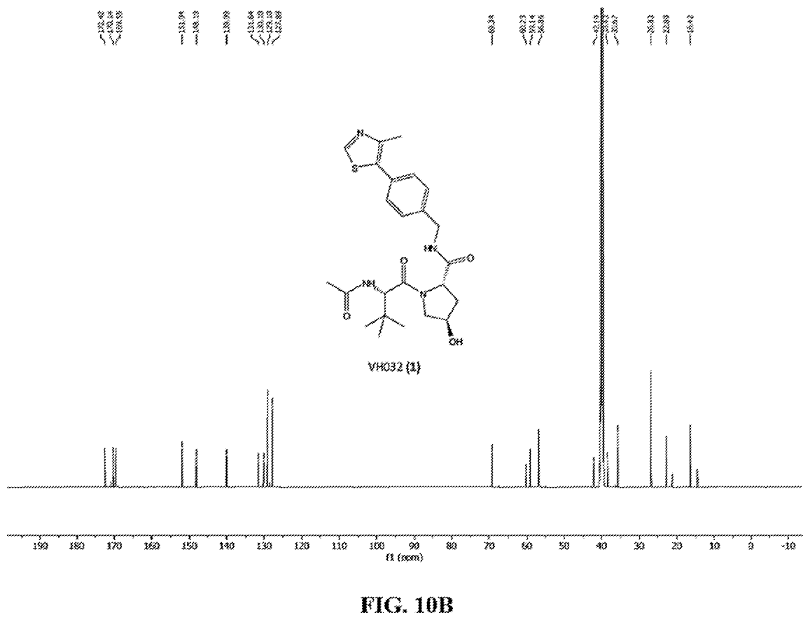

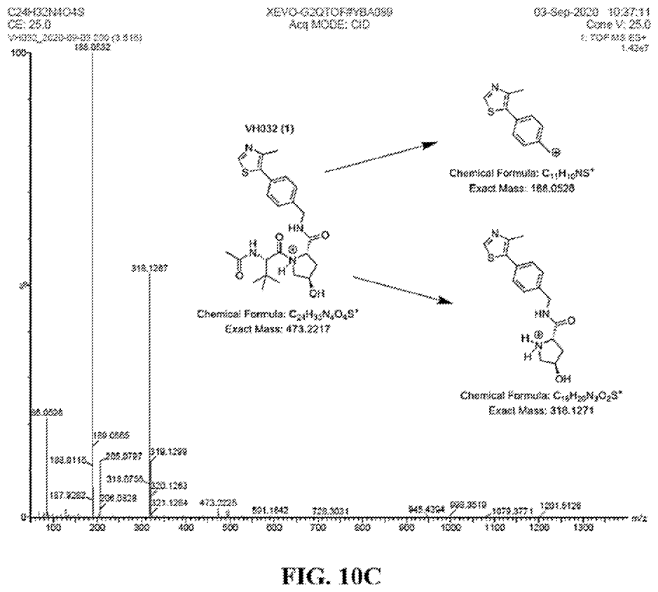

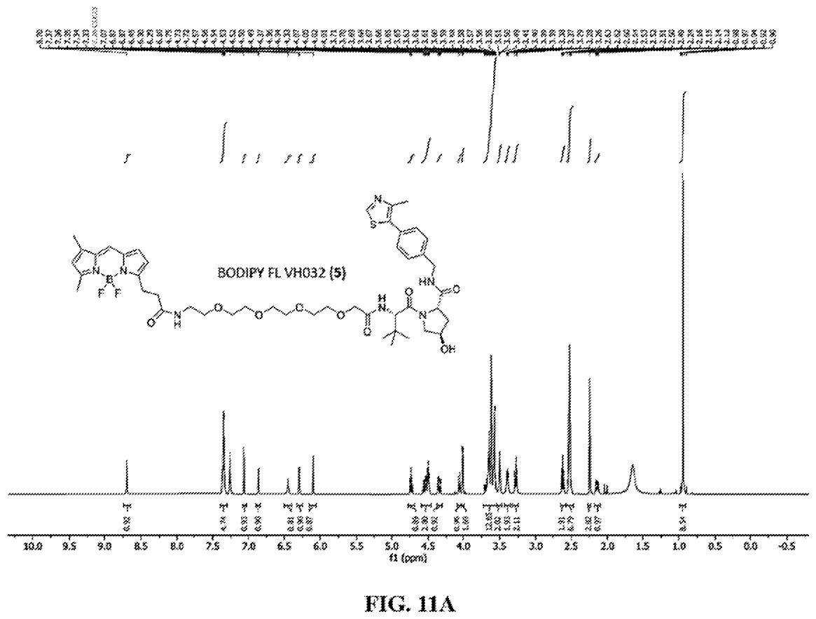

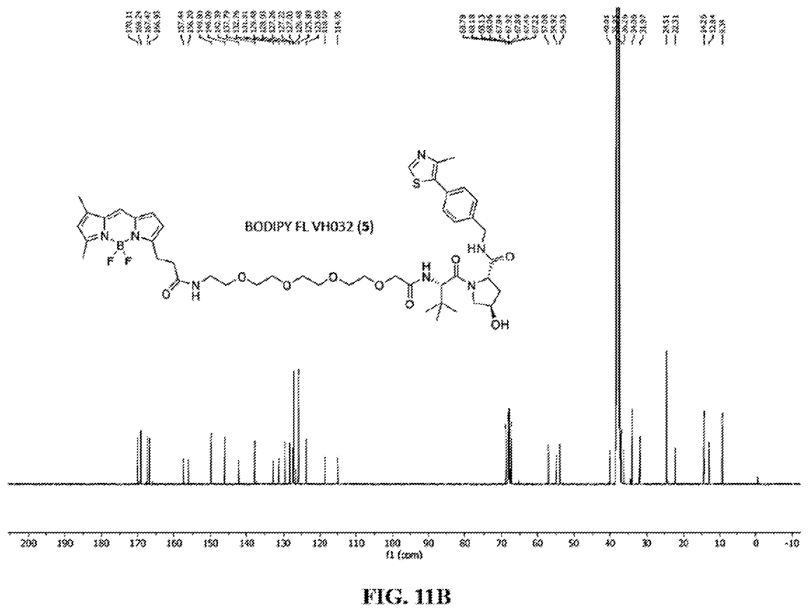

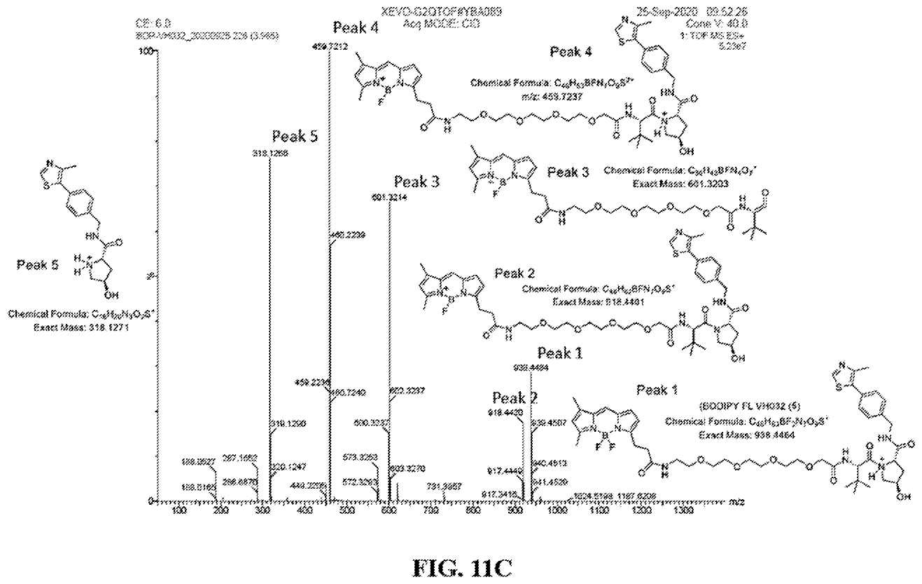

Next, the FP assay was applied with the established condition of 10 nM BODIPY FL VH032 (5), 100 nM GST-VCB, and a 90-min incubation time to characterize pVHL ligands for their binding affinities. The negative control (DMSO), positive control [VH298 (2, 30 μM)] and dilutions (1-to-3 dilutions, a concentration range of 2.1 pM to 30 μM) of the same panel of pVHL ligands and non-pVHL ligands as tested in the TR-FRET assay were tested in the FP assay. The negative control (DMSO) and the positive control [VH298 (2, 30 μM)] had corresponding FP signal of 143.75 and 14.5 mP (FIG. 9A) and respective FP signal fold change to the positive control VH298 (2, 30 μM) of 9.91 and 1.00-fold (FIG. 9B). The FP signal fold change of 9.91-fold between the negative control (DMSO) and the positive control [VH298 (2, 30 μM)] was only slightly less than that of the TR-FRET assay (13.88-fold).

The dose response curves of the pVHL ligands VH032 (1), VH298 (2), VH032 amine (6), Me-VH032 amine (7), BOC-VH032 (8), and VH032 phenol (9) in the FP assay are summarized in FIG. 9C, and those for the BRD-VHL PROTAC ligands MZ1 (3) and dBET1 (12), together with their PROTAC components (+)-JQ1 (4), VH032-PEG4-amine (10) and Thalidomide-4′-oxyacetamido-alkylC4-amine (11), as well as the positive control VH298 (2) in FIG. 9D. pVHL ligands VH032 (1), VH298 (2), MZ1 (3), BOC-VH032 (8), VH032 phenol (9), and VH032-PEG4-amine (10) had respective pVHL inhibitory IC₅₀ values of 352.2 nM, 288.2 nM, 226.2 nM, 16.3 μM, 212.5 nM, and 430.8 nM and Kᵢ values of 142.1 nM, 110.4 nM, 79.7 nM, 8.0 μM, 77.9 nM, and 181.0 nM. VH032 amine (6) had a maximal FP % Inhibition of only 36.6% at the maximal tested concentration of 30 μM; therefore, its IC₅₀ or Kᵢ value could not be determined with the FP assay. However, it had corresponding TR-FRET IC₅₀ and Kᵢ values of 13.3 μM and 5.7 μM (FIG. 6A), thus, the TR-FRET assay is more sensitive than the FP assay in detecting pVHL ligands with lower binding affinity. Furthermore, the TR-FRET assay is more robust than the FP assay in testing ligands which may interfere with the assays. For example, in the FP assay, Me-VH032 amine (7) (the purple curve with purple solid inversed triangle in FIG. 9C) disturbed the assay detection at concentration at or higher than 370 nM; however, such assay interference was not observed in the TR-FRET assay (FIG. 6A). A respective IC₅₀ and Kᵢ values of 7.9 μM and 3.4 μM for Me-VH032 amine (7) was determined in the TR-FRET assay without any interference observed. However, the FP assay is as selective as the TR-FRET assay, because the non-pVHL ligands (+)-JQ1 (4), Thalidomide-4′-oxyacetamido-alkylC4-amine (11) and dBET1 (12) did not show pVHL inhibitory activity in the FP assay.

Referring to FIG. 9A, the FP assay performance of DMSO (the negative control group), VH298 (2, 30 μM) (the positive control group), and DMSO+without GST-VCB (the background control group) is shown.

Referring to FIG. 9B, the FP signal fold change [to VH298 (2, 30 μM, the positive control group] of DMSO (the negative control group), VH298 (2, 30 μM) (the positive control group) and DMSO+without GST-VCB (the background control group) is shown.

Referring to FIG. 9C, the FP dose response curves of pVHL ligands VH032 (1), VH298 (2), VH032 amine (6), Me-VH032 amine (7), BOC-VH032 (8), and VH032 phenol (9) are shown.

Referring to FIG. 9D, the FP dose response curves of pVHL ligands VH298 (2), MZ1 (3), VH032-PEG4-amine (10), and non-pVHL ligands of (+)-JQ1 (4, a BRD ligand), Thalidomide-4′-oxyacetamido-alkylC4-amine (11, a cereblon E3 ligase ligand), and dBET1 (12, a BRD-CRBN PROTAC) are shown.

j. Comparison of the Newly Developed pVHL TR-FRET and FP Assays to the Reported pVHL FP Assay

Fluorescently labeled peptides derived from HIF-1α protein have been used to characterize pVHL ligands in FP assays (Buckley, et al. (2012) Targeting the von Hippel-Lindau E3 ubiquitin ligase using small molecules to disrupt the VHL/HIF-1α interaction. J Am Chem Soc 134, 4465-4468; Frost, J, et al. (2016) Potent and selective chemical probe of hypoxic signalling downstream of HIF-α hydroxylation via VHL inhibition. Nat Commun 7, 13312). Two versions of FAM-HIF-1α peptides, FAM-DEALAHyp-YIPD (10-mer, MW: 1477.48), and FAM-DEALAHyp-YIPMDDDFQLRSF (19-mer, M+H: 2617.167) have been reported. In one report (Buckley, et al. (2012) Targeting the von Hippel-Lindau E3 ubiquitin ligase using small molecules to disrupt the VHL/HIF-1α interaction. J Am Chem Soc 134, 4465-4468), the FAM-DEALAHyp-YIPD (10-mer) and FAM-DEALAHyp-YIPMDDDFQLRSF (19-mer) had respective K_{d} of 560 nM and 36 nM with FP assays. The FAM-DEALAHyp-YIPMDDDFQLRSF (19-mer) has also been reported with a K_{d} of 3 nM under another FP assay condition (Frost, J, et al. (2016) Potent and selective chemical probe of hypoxic signalling downstream of HIF-α hydroxylation via VHL inhibition. Nat Commun 7, 13312). The small molecule probe BODIPY FL VH032 (5, MW: 937.91 Da) that was developed had a K_{d} value of 3.01 nM in the TR-FRET assay, which is similar to the reported K_{d} value of 3 nM of the FAM-DEALAHyp-YIPMDDDFQLRSF (19-mer, M+H: 2617.167) in one of the reported FP assay. However, BODIPY FL VH032 (5) is smaller in size based on its molecular weight, so it's impressive for its K_{d} value to be similar to that of a big peptide. The BODIPY FL VH032 (5) had a FP K_{d} value of 100.8 nM, which is better than the FP K_{d} value of 560 nM for the 10-mer FAM-DEALAHyp-YIPD HIF-1α peptide (10-mer, MW: 1477.48), although the latter one was derived from protein with a bigger molecular size.

Three pVHL ligands VH032 (1), VH298 (2) and BOC-VH032 (8) were tested in the FAM-HIF-1α peptide (19-mer)-based FP assay (the FP assay that reported the highest affinity peptide probe) and in the new BODIPY FL VH032 (5)-based TR-FRET and FP assays, and their activities summarized in Table 1. Although the affinity rank order [VH298 (2), VH032 (1), and BOC-VH032 (8) from high to low] is the same among the three assays, the BODIPY FL VH032 (5)-based TR-FRET assay is the most sensitive. The most potent inhibitor VH298 (2) had respective pVHL inhibitory activity values of 80 nM (K_{d}), 18.9 nM (Kᵢ) and 110.4 nM (Kᵢ) for the FAM-HIF-1α peptide (19-mer)-mediated FP assay, the BODIPY FL VH032 (5)-mediated TR-FRET and FP assay. For measuring the activity of VH298 (2), the BODIPY FL VH032 (5)-based TR-FRET assay was more sensitive (4.23-fold) than the FAM-HIF-1α peptide (19-mer)-based FP assay. Similar sensitivity improvements of the TR-FRET assay were also observed for VH032 (1) and BOC-VH032 (8) (4.49-fold and 3.09-fold, respectively). In addition, less VCB protein (2 nM) was consumed with the TR-FRET assay, compared to the best FP assay reported that consumed nM VCB protein. However, the BODIPY FL VH032 (5)-based FP assay just had comparable sensitivity to the reported FP assay using FAM-HIF-1α peptide (19-mer) as the probe and reported the highest affinity for that peptide probe.

The activities of the pVHL ligands active in both BODIPY FL VH032 (5)-mediated TR-FRET and FP assays are also summarized (Table 1). The TR-FRET assay is more sensitive (with lower Kᵢ values) than the FP assay for all the active ligands including VH032 (1), VH298 (2), MZ1 (3), BOC-VH032 (8), VH032 phenol (9), and VH032-PEG4-amine (10). The most potent ligand tested with the TR-FRET assay was MZ1 (3) with a Kᵢ value of 6.3 nM. The weakest pVHL ligand tested with the TR-FRET assay was VH032 amine (6) with a Kᵢ value of 5.7 μM. Thus, the TR-FRET assay was able to detect compounds with various affinity, at least ranging from 6.3 nM to 5.7 μM in Kᵢ values based on the tested ligands. The activity rank orders are generally similar for those pVHL ligands tested with the two assays, although with slight differences observed. It has been reported that TR-FRET assay and FP assay may give slightly different activity rank orders when a common set of ligands are tested (Newman, M., and Josiah, S. (2004) Utilization of fluorescence polarization and time resolved fluorescence resonance energy transfer assay formats for SAR studies: Src kinase as a model system. J Biomol Screen 9, 525-532; Cashman, J. R., et al. (2010) Inhibition of Bfl-1 with N-aryl maleimides. Bioorg Med Chem Lett 20, 6560-6564; Klink, T. A., et al. (2008) Evaluating PI3 kinase isoforms using Transcreener ADP assays. J Biomol Screen 13, 476-485), but TR-FRET assay was believed to be superior than FP assay because of its lower assay variability, lower nonspecific interference, and better correlation to cell-based assay (Raucy, J. L., and Lasker, J. M. (2010) Current in vitro high throughput screening approaches to assess nuclear receptor activation. Curr Drug Metab 11, 806-814).

TABLE 1
COMPARISON OF THE BEST REPORTED PVHL FP ASSAY AND THE
PVHL TR-FRET AND FP ASSAYS DEVELOPED IN THIS REPORT

| | FP assay (Soares, et al.) | TR-FRET assay^{a} | FP assay^{a} |
|---|---|---|---|
| Probe and its K_{d} | FAM-HIF-1α | BODIPY FL VH032 | BODIPY FL VH032 |
| | FP assay (Soares, et al.) | TR-FRET assay^{a} | FP assay^{a} |
| | peptide (19-mer), 3 | (5) | (5) |
| | nM (K_{d}) | 3.01 nM (K_{d}) | 100.8 nM (K_{d}) |
| Reagent condition | 10 nM FAM-HIF-1α | 4 nM BODIPY FL | 10 nM BODIPY FL |
| | 15 nM VCB | VH032 (5) | VH032 (5) |
| | | 2 nM GST-VCB | 100 nM GST-VCB |
| | | 2 nM Tb-anti-GST | |
| VH032 (1) | 150 nM (K_{d}) | 33.4 nM (Kᵢ) | 142.1 nM (Kᵢ) |
| | | (4.49-fold) | |
| VH298 (2) | 80 nM (K_{d}) | 18.9 nM (Kᵢ) | 110.4 nM (Kᵢ) |
| | | (4.23-fold) | |
| MZ1 (3) | NA^{b} | 6.3 nM | 79.7 nM |
| BOC-VH032 (8) | 6.5 μM (K_{d}) | 2.1 μM (Kᵢ) | 8.0 μM (Kᵢ) |
| | | (3.09-fold) | |
| VH032 phenol (9) | NA | 14.6 nM (Kᵢ) | 77.9 nM (Kᵢ) |
| VH032-PEG4- | NA | 6.8 nM (Kᵢ) | 181.0 nM (Kᵢ) |
| amine (10) | | | |

^{a}assay developed in this report;
^{b}NA, not available.

In conclusion, the BODIPY FL VH032 (5) has been developed as the first small molecule fluorescent probe with high affinity to pVHL (a K_{d} value of 3.01 nM in a TR-FRET assay). The BODIPY FL VH032 (5)-mediated pVHL TR-FRET binding assay is more sensitive than reported FP assays and the FP assay developed based on the same BODIPY FL VH032 (5) as the fluorescent probe, and it is less susceptible to interference of certain as observed in an FP assay. Both BODIPY FL VH032 (5)-mediated TR-FRET and FP assays only selectively detect pVHL ligands. It has been reported that assays based on a high-affinity probe are more sensitive in detecting the binding of ligands with a wide range of inhibitory potency (Huang, X. (2003) Fluorescence polarization competition assay: the range of resolvable inhibitor potency is limited by the affinity of the fluorescent ligand. J Biomol Screen 8, 34-38). The high-affinity VHL fluorescent probe BODIPY FL VH032 (5)-based TR-FRET assay is sensitive, selective, resistant to assay interference, and capable of detecting ligands with a wide range of activity, and is therefore suitable for identification and characterization of VHL ligands in large scale screenings.

### H. REFERENCES

Kibel, A., Iliopoulos, O., DeCaprio, J. A., and Kaelin, W. G., Jr. (1995) Binding of the von Hippel-Lindau tumor suppressor protein to Elongin B and C. Science 269, 1444-1446.
Stebbins, C. E., Kaelin, W. G., Jr., and Pavletich, N. P. (1999) Structure of the VHL-ElonginC-ElonginB complex: implications for VHL tumor suppressor function. Science 284, 455-461.
Maxwell, P. H., Wiesener, M. S., Chang, G. W., Clifford, S. C., Vaux, E. C., Cockman, M. E., Wykoff, C. C., Pugh, C. W., Maher, E. R., and Ratcliffe, P. J. (1999) The tumour suppressor protein VHL targets hypoxia-inducible factors for oxygen-dependent proteolysis. Nature 399, 271-275.
Buckley, D. L., Van Molle, I., Gareiss, P. C., Tae, H. S., Michel, J., Noblin, D. J., Jorgensen, W. L., Ciulli, A., and Crews, C. M. (2012) Targeting the von Hippel-Lindau E3 ubiquitin ligase using small molecules to disrupt the VHL/HIF-1α interaction. J Am Chem Soc 134, 4465-4468.
Buckley, D. L., Gustafson, J. L., Van Molle, I., Roth, A. G., Tae, H. S., Gareiss, P. C., Jorgensen, W. L., Ciulli, A., and Crews, C. M. (2012) Small-molecule inhibitors of the interaction between the E3 ligase VHL and HIF1α. Angew Chem Int Ed Engl 51, 11463-11467.
Van Molle, I., Thomann, A., Buckley, D. L., So, E. C., Lang, S., Crews, C. M., and Ciulli, A. (2012) Dissecting fragment-based lead discovery at the von Hippel-Lindau protein:hypoxia inducible factor 1α protein-protein interface. Chem Biol 19, 1300-1312.
Galdeano, C., Gadd, M. S., Soares, P., Scaffidi, S., Van Molle, I., Birced, I., Hewitt, S., Dias, D. M., and Ciulli, A. (2014) Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities. J Med Chem 57, 8657-8663.
Frost, J., Galdeano, C., Soares, P., Gadd, M. S., Grzes, K. M., Ellis, L., Epemolu, O., Shimamura, S., Bantscheff, M., Grandi, P., Read, K. D., Cantrell, D. A., Rocha, S., and Ciulli, A. (2016) Potent and selective chemical probe of hypoxic signalling downstream of HIF-α hydroxylation via VHL inhibition. Nat Commun 7, 13312.
Min, J. H., Yang, H., Ivan, M., Gertler, F., Kaelin, W. G., Jr., and Pavletich, N. P. (2002) Structure of an HIF-1alpha-pVHL complex: hydroxyproline recognition in signaling. Science 296, 1886-1889.
Hon, W. C., Wilson, M. I., Harlos, K., Claridge, T. D., Schofield, C. J., Pugh, C. W., Maxwell, P. H., Ratcliffe, P. J., Stuart, D. I., and Jones, E. Y. (2002) Structural basis for the recognition of hydroxyproline in HIF-1 alpha by pVHL. Nature 417, 975-978.
Wang, Y., Jiang, X., Feng, F., Liu, W., and Sun, H. (2020) Degradation of proteins by PROTACs and other strategies. Acta Pharm Sin B 10, 207-238.
Gadd, M. S., Testa, A., Lucas, X., Chan, K. H., Chen, W., Lamont, D. J., Zengerle, M., and Ciulli, A. (2017) Structural basis of PROTAC cooperative recognition for selective protein degradation. Nat Chem Biol 13, 514-521.
Filippakopoulos, P., Qi, J., Picaud, S., Shen, Y., Smith, W. B., Fedorov, O., Morse, E. M., Keates, T., Hickman, T. T., Felletar, I., Philpott, M., Munro, S., McKeown, M. R., Wang, Y., Christie, A. L., West, N., Cameron, M. J., Schwartz, B., Heightman, T. D., La Thangue, N., French, C. A., Wiest, O., Kung, A. L., Knapp, S., and Bradner, J. E. (2010) Selective inhibition of BET bromodomains. Nature 468, 1067-1073.
Soares, P., Gadd, M. S., Frost, J., Galdeano, C., Ellis, L., Epemolu, O., Rocha, S., Read, K. D., and Ciulli, A. (2018) Group-Based Optimization of Potent and Cell-Active Inhibitors of the von Hippel-Lindau (VHL) E3 Ubiquitin Ligase: Structure-Activity Relationships Leading to the Chemical Probe (2S,4R)-1-((S)-2-(1-Cyanocyclopropanecarboxamido)-3,3-dimethylbutanoyl)-4-hydroxy-N-(4-(4-methylthiazol-5-yl)benzyl)pyrrolidine-2-carboxamide (VH298). J Med Chem 61, 599-618.
Testa, A., Lucas, X., Castro, G. V., Chan, K. H., Wright, J. E., Runcie, A. C., Gadd, M. S., Harrison, W. T. A., Ko, E. J., Fletcher, D., and Ciulli, A. (2018) 3-Fluoro-4-hydroxyprolines: Synthesis, Conformational Analysis, and Stereoselective Recognition by the VHL E3 Ubiquitin Ligase for Targeted Protein Degradation. J Am Chem Soc 140, 9299-9313.
Crew, A. P., Raina, K., Dong, H., Qian, Y., Wang, J., Vigil, D., Serebrenik, Y. V., Hamman, B. D., Morgan, A., Ferraro, C., Siu, K., Neklesa, T. K., Winkler, J. D., Coleman, K. G., and Crews, C. M. (2018) Identification and Characterization of Von Hippel-Lindau-Recruiting Proteolysis Targeting Chimeras (PROTACs) of TANK-Binding Kinase 1. J Med Chem 61, 583-598.
Zoppi, V., Hughes, S. J., Maniaci, C., Testa, A., Gmaschitz, T., Wieshofer, C., Koegl, M., Riching, K. M., Daniels, D. L., Spallarossa, A., and Ciulli, A. (2019) Iterative Design and Optimization of Initially Inactive Proteolysis Targeting Chimeras (PROTACs) Identify VZ185 as a Potent, Fast, and Selective von Hippel-Lindau (VHL) Based Dual Degrader Probe of BRD9 and BRD7. J Med Chem 62, 699-726.
Lea, W. A., and Simeonov, A. (2011) Fluorescence polarization assays in small molecule screening. Expert Opin Drug Discov 6, 17-32.
Parker, G. J., Law, T. L., Lenoch, F. J., and Bolger, R. E. (2000) Development of high throughput screening assays using fluorescence polarization: nuclear receptor-ligand-binding and kinase/phosphatase assays. J Biomol Screen 5, 77-88.
Du, Y., Nikolovska-Coleska, Z., Qui, M., Li, L., Lewis, I., Dingledine, R., Stuckey, J. A., Krajewski, K., Roller, P. P., Wang, S., and Fu, H. (2011) A dual-readout F2 assay that combines fluorescence resonance energy transfer and fluorescence polarization for monitoring bimolecular interactions. Assay Drug Dev Technol 9, 382-393.
Moerke, N. J. (2009) Fluorescence Polarization (FP) Assays for Monitoring Peptide-Protein or Nucleic Acid-Protein Binding. Curr Protoc Chem Biol 1, 1-15.
Lin, W., and Chen, T. (2018) Using TR-FRET to Investigate Protein-Protein Interactions: A Case Study of PXR-Coregulator Interaction. Adv Protein Chem Struct Biol 110, 31-63.
Lin, W., Liu, J., Jeffries, C., Yang, L., Lu, Y., Lee, R. E., and Chen, T. (2014) Development of BODIPY FL vindoline as a novel and high-affinity pregnane X receptor fluorescent probe. Bioconjug Chem 25, 1664-1677.
Lin, W., and Chen, T. (2013) A vinblastine fluorescent probe for pregnane X receptor in a time-resolved fluorescence resonance energy transfer assay. Anal Biochem 443, 252-260.
Thorne, N., Auld, D. S., and Inglese, J. (2010) Apparent activity in high-throughput screening: origins of compound-dependent assay interference. Curr Opin Chem Biol 14, 315-324.
Glickman, J. F., Wu, X., Mercuri, R., Illy, C., Bowen, B. R., He, Y., and Sills, M. (2002) A comparison of ALPHAScreen, TR-FRET, and TRF as assay methods for FXR nuclear receptors. J Biomol Screen 7, 3-10.
Lai, A. C., Toure, M., Hellerschmied, D., Salami, J., Jaime-Figueroa, S., Ko, E., Hines, J., and Crews, C. M. (2016) Modular PROTAC Design for the Degradation of Oncogenic BCR-ABL. Angew Chem Int Ed Engl 55, 807-810.
Raina, K., Lu, J., Qian, Y., Altieri, M., Gordon, D., Rossi, A. M., Wang, J., Chen, X., Dong, H., Siu, K., Winkler, J. D., Crew, A. P., Crews, C. M., and Coleman, K. G. (2016) PROTAC-induced BET protein degradation as a therapy for castration-resistant prostate cancer. Proc Natl Acad Sci USA 113, 7124-7129.
Maniaci, C., Hughes, S. J., Testa, A., Chen, W., Lamont, D. J., Rocha, S., Alessi, D. R., Romeo, R., and Ciulli, A. (2017) Homo-PROTACs: bivalent small-molecule dimerizers of the VHL E3 ubiquitin ligase to induce self-degradation. Nat Commun 8, 830.
Winter, G. E., Buckley, D. L., Paulk, J., Roberts, J. M., Souza, A., Dhe-Paganon, S., and Bradner, J. E. (2015) DRUG DEVELOPMENT. Phthalimide conjugation as a strategy for in vivo target protein degradation. Science 348, 1376-1381.
Chan, L. C., and Cox, B. G. (2007) Kinetics of Amide Formation through Carbodiimide/N-Hydroxybenzotriazole (HOBt) Couplings. The Journal of Organic Chemistry 72, 8863-8869.
Cheng, Y., and Prusoff, W. H. (1973) Relationship between the inhibition constant (K1) and the concentration of inhibitor which causes 50 percent inhibition (I50) of an enzymatic reaction. Biochem Pharmacol 22, 3099-3108.
Nikolovska-Coleska, Z., Wang, R., Fang, X., Pan, H., Tomita, Y., Li, P., Roller, P. P., Krajewski, K., Saito, N. G., Stuckey, J. A., and Wang, S. (2004) Development and optimization of a binding assay for the XIAP BIR3 domain using fluorescence polarization. Anal Biochem 332, 261-273.
Newman, M., and Josiah, S. (2004) Utilization of fluorescence polarization and time resolved fluorescence resonance energy transfer assay formats for SAR studies: Src kinase as a model system. J Biomol Screen 9, 525-532.
Cashman, J. R., MacDonald, M., Ghirmai, S., Okolotowicz, K. J., Sergienko, E., Brown, B., Garcia, X., Zhai, D., Dahl, R., and Reed, J. C. (2010) Inhibition of Bfl-1 with N-aryl maleimides. Bioorg Med Chem Lett 20, 6560-6564.
Klink, T. A., Kleman-Leyer, K. M., Kopp, A., Westermeyer, T. A., and Lowery, R. G. (2008) Evaluating PI3 kinase isoforms using Transcreener ADP assays. J Biomol Screen 13, 476-485.
Raucy, J. L., and Lasker, J. M. (2010) Current in vitro high throughput screening approaches to assess nuclear receptor activation. Curr Drug Metab 11, 806-814.
Huang, X. (2003) Fluorescence polarization competition assay: the range of resolvable inhibitor potency is limited by the affinity of the fluorescent ligand. J Biomol Screen 8, 34-38.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound having a structure represented by a formula: 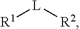
wherein L is a linker;
wherein R¹ is a residue of a fluorophore, a residue of biotin, or a residue of a biotin derivative; and
wherein R² is a residue of a von Hippel-Lindau protein (pVHL) ligand.

2. The compound of claim 1, wherein R¹ is a residue of biotin or a biotin derivative.

3. (canceled)

4. The compound of claim 2, wherein the biotin derivative is biocytin or desthiobiotin.

5. The compound of claim 1, wherein R¹ is a residue of a fluorophore.

6. (canceled)

7. The compound of claim 5, wherein the fluorophore is a 4,4-difluoro-4-bora-3a,4a-diaza-s-indacene (BODIPY) fluorophore.

8. The compound of claim 5, wherein the BODIPY fluorophore is selected from:

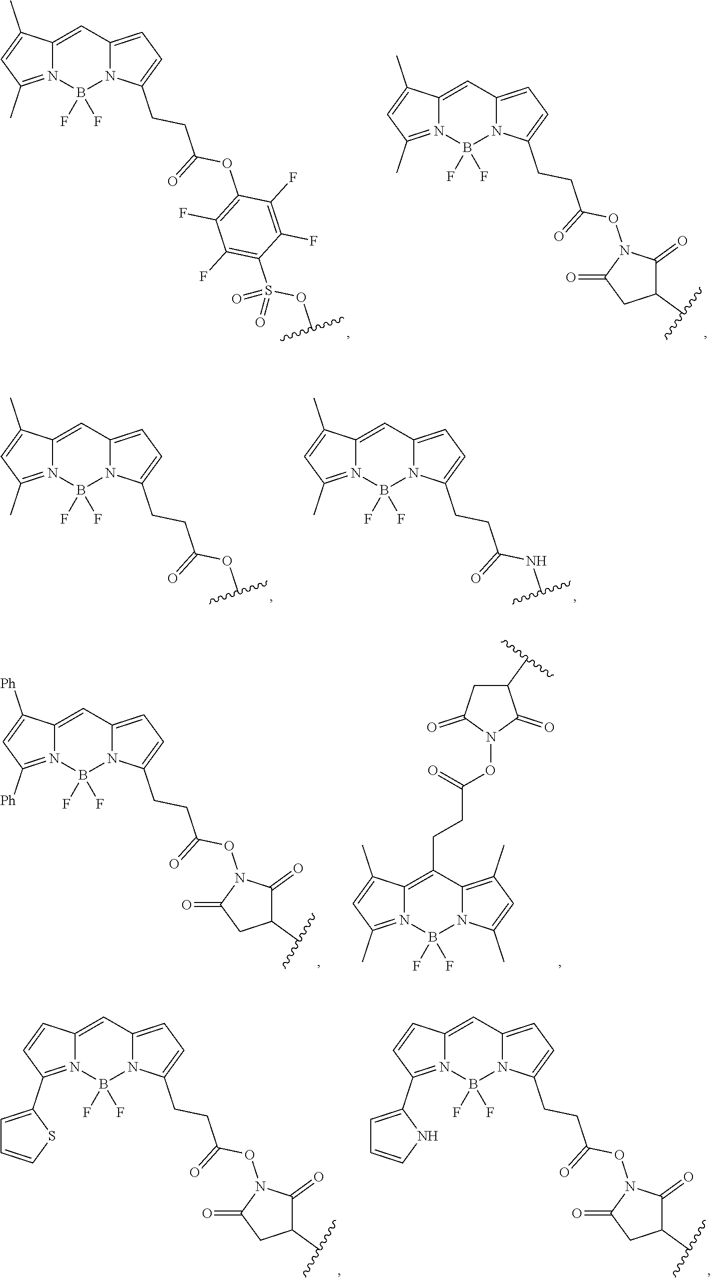

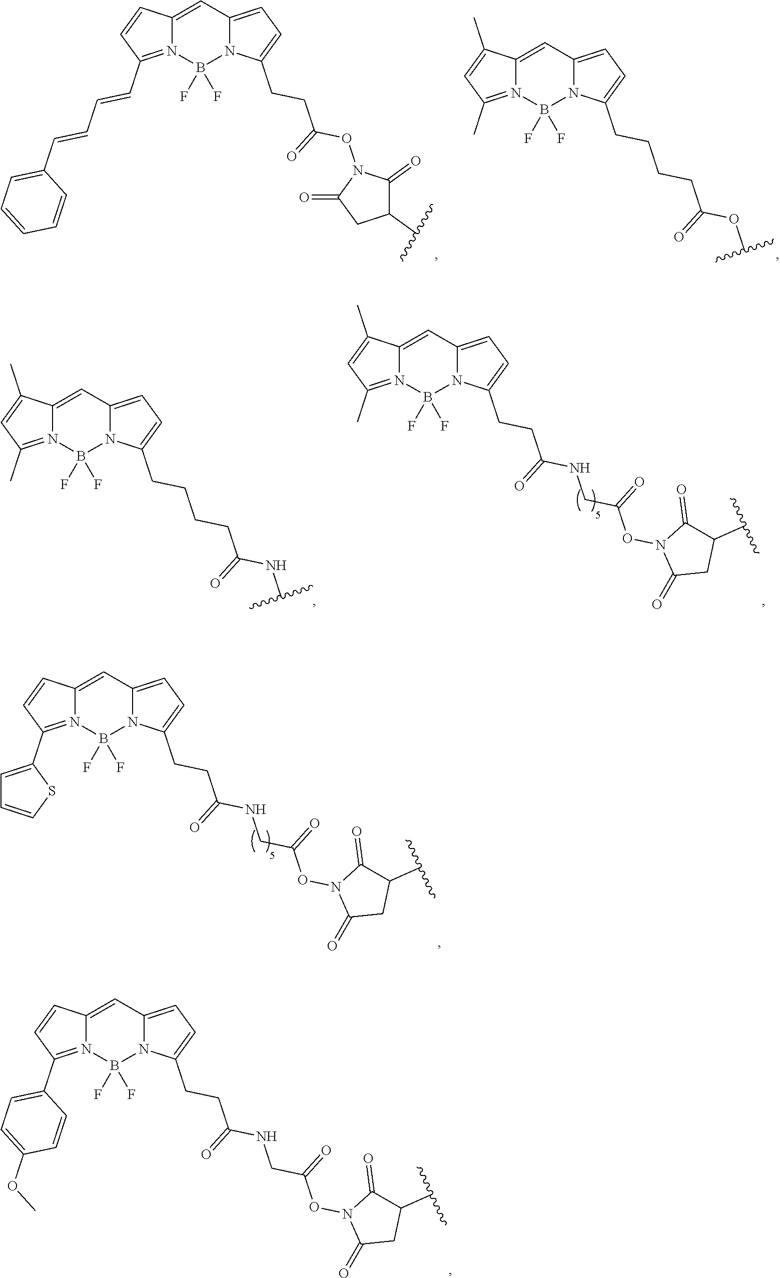

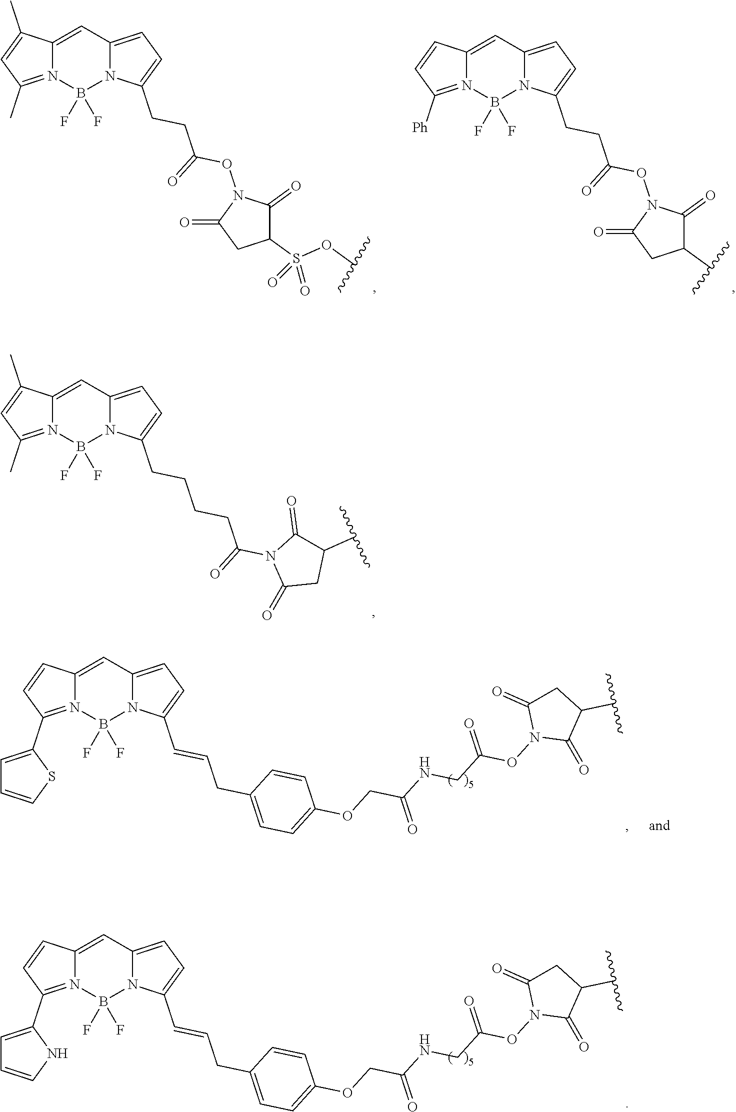

9. The compound of claim 7, wherein the BODIPY fluorophore is:

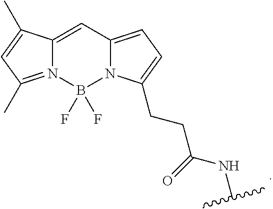

10. The compound of claim 1, wherein L is selected from C2-C15 alkyl and —(CH₂CH₂O)ₙ, and wherein n is selected from 1, 2, 3, 4, 5, 6, 7, and 8.

11-16. (canceled)

17. The compound of claim 1, wherein the residue of the pVHL ligand has a structure represented by a formula: 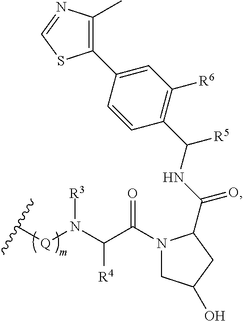
wherein m is 0 or 1;
wherein Q, when present, is —OC(O)—, —C(R^{10a})(R^{10b})C(O)—, —OC(R^{10a})(R^{10b})C(O)—, —C(R^{10a})(R^{10b})C(O)C(cyclopropyl)C(O)—, —C(R^{10a})(R^{10b})C(O)N(R^{11a})CH₂CH(R^{11b})C(O)—, —C(C3-C4 cycloalkyl)C(O)—, —NH(CH₂CH₂O)_{q}CH₂C(O)—, —NHCH₂C(cyclopropyl)C(O)—, or —CH₂C(O)N(R¹²)CH(R¹³)C(O)—;
wherein q, when present, is 1, 2, 3, 4, 5, or 6;
wherein each of R^{10a} and R^{10b}, when present, is independently hydrogen or C1-C4 alkyl;
or wherein each of R^{10a} and R^{10b}, when present, are covalently bound, and, together comprise a C3-C4 cycloalkyl or a C2-C3 heterocycloalkyl;
or wherein R¹⁰, when present, is covalently bound to R³, and, together with the intermediate atoms, comprises a 5-membered heterocycle;
wherein each of R^{11a} and R^{11b}, when present, are covalently bound, and, together with the intermediate atoms, comprise a 4-membered heterocycle;
wherein R¹², when present, is hydrogen; and
wherein R¹³, when present, is C1-C4 alkyl, —CH₂C₆H₅, or —C₆H₅;
or wherein each of R¹² and R¹³, when present, are covalently bound, and, together with the intermediate atoms, comprise an 10-membered heterocycloalkyl;
wherein R³ is hydrogen or C1-C4 alkyl; and
wherein R⁴ is a C1-C4 alkyl, C1-C4 hydroxyalkyl, or C₆H₅;
or wherein each of R³ and R⁴ are covalently bound, and, together with the intermediate atoms, comprise a 5- or 6-membered heterocycle having 0 or 1 —OH group;
or wherein each of R³ and R¹⁰, when present, are covalently bound, and, together with the intermediate atoms, comprise a 5-membered heterocycle;
wherein R⁵ is hydrogen or methyl; and
wherein R⁶ is hydrogen, —OH, or C1-C4 alkyl halide.

18. The compound of claim 17, wherein each of R³, R⁵, and R⁶ is hydrogen.

19. The compound of claim 17, wherein R⁴ is tert-butyl.

20-23. (canceled)

24. The compound of claim 17, wherein the residue of the pVHL ligand has a structure represented by a formula:

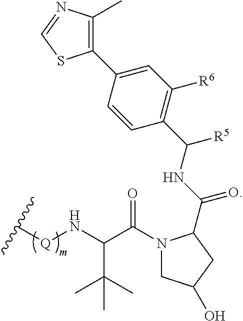

25. (canceled)

26. The compound of claim 17, wherein the residue of the pVHL ligand has a structure selected from:

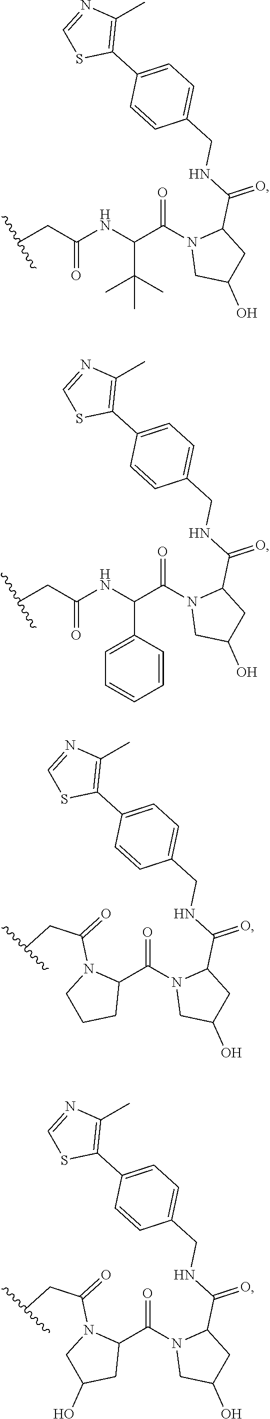

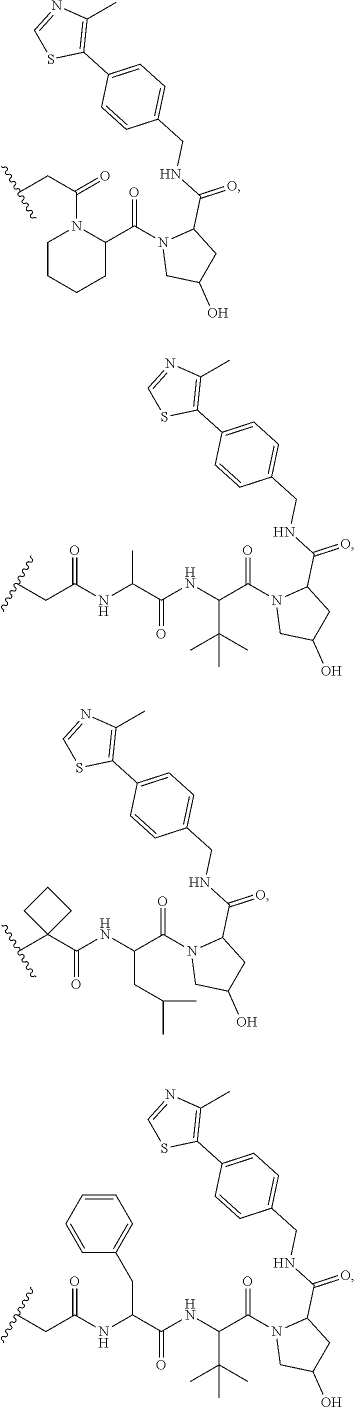 [SMILES: *CC(=O)N1CCCCC1C(=O)N1CC(O)CC1C(=O)NCc1ccc(-c2scnc2C)cc1 ; *CC(=O)NC(C)C(=O)NC(C(=O)N1CC(O)CC1C(=O)NCc1ccc(-c2scnc2C)cc1)C(C)(C)C ; *C1(C(=O)NC(CC(C)C)C(=O)N2CC(O)CC2C(=O)NCc2ccc(-c3scnc3C)cc2)CCC1 ; *CC(=O)NC(Cc1ccccc1)C(=O)NC(C(=O)N1CC(O)CC1C(=O)NCc1ccc(-c2scnc2C)cc1)C(C)(C)C]

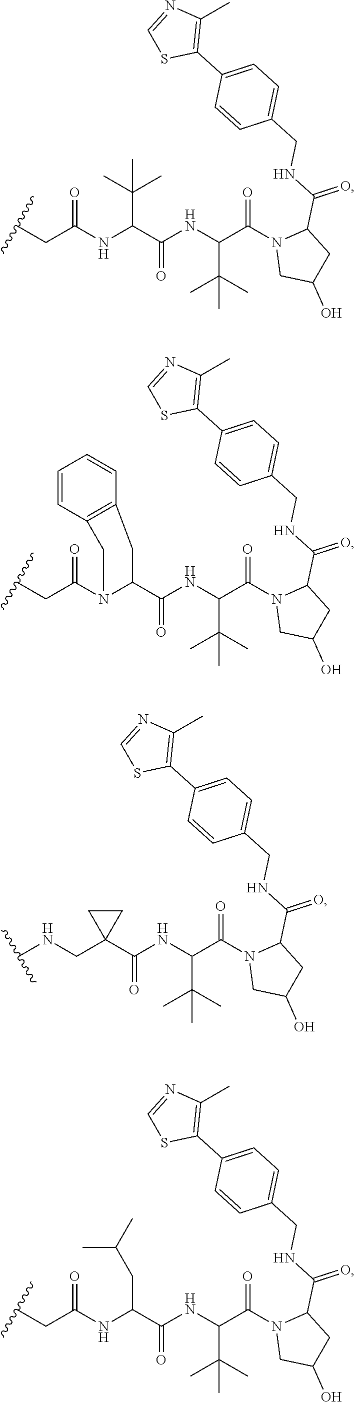 [SMILES: *CC(=O)NC(C(=O)NC(C(=O)N1CC(O)CC1C(=O)NCc1ccc(-c2scnc2C)cc1)C(C)(C)C)C(C)(C)C ; *CC(=O)N1Cc2ccccc2CC1C(=O)NC(C(=O)N1CC(O)CC1C(=O)NCc1ccc(-c2scnc2C)cc1)C(C)(C)C ; *NCC1(C(=O)NC(C(=O)N2CC(O)CC2C(=O)NCc2ccc(-c3scnc3C)cc2)C(C)(C)C)CC1 ; *CC(=O)NC(CC(C)C)C(=O)NC(C(=O)N1CC(O)CC1C(=O)NCc1ccc(-c2scnc2C)cc1)C(C)(C)C]

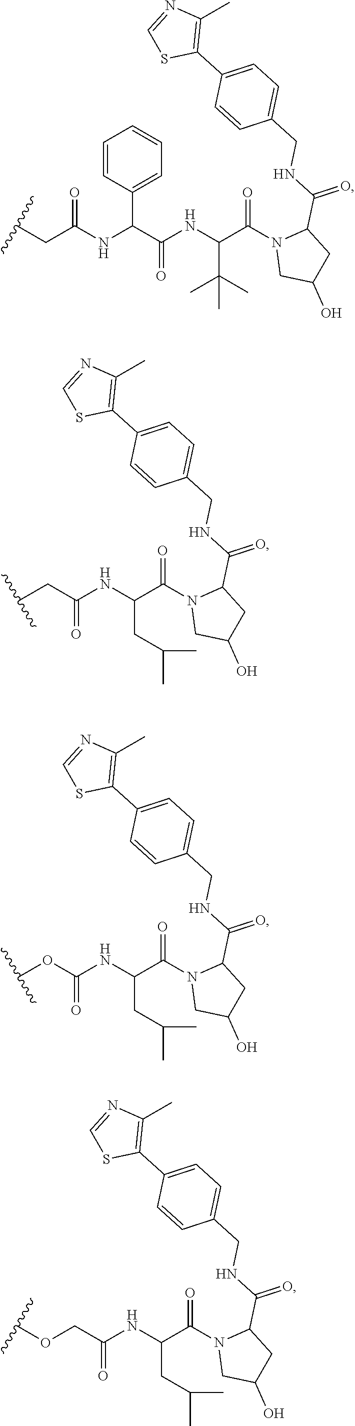

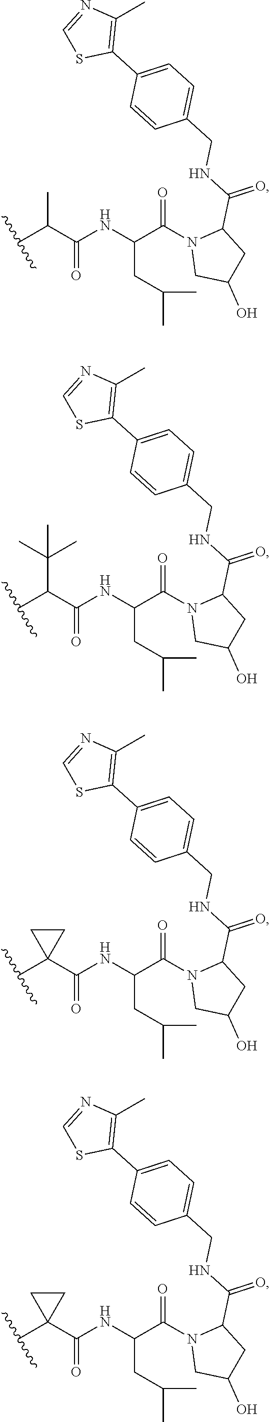

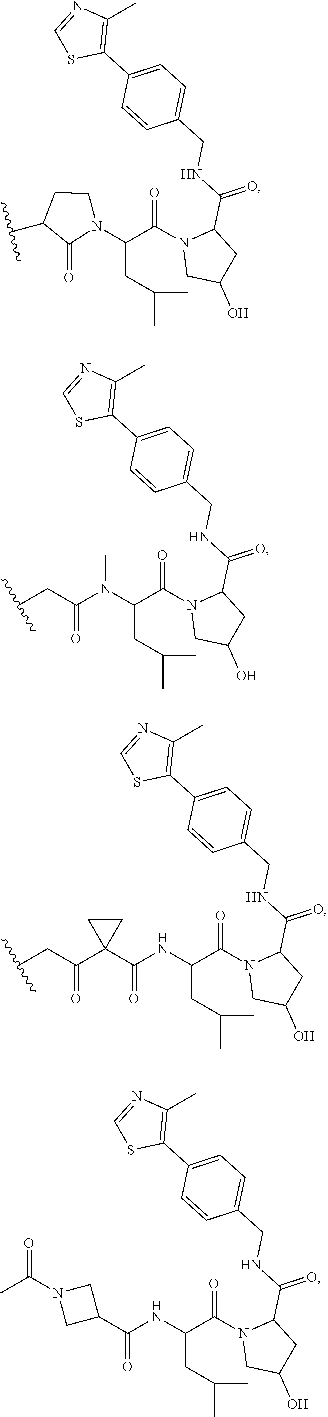

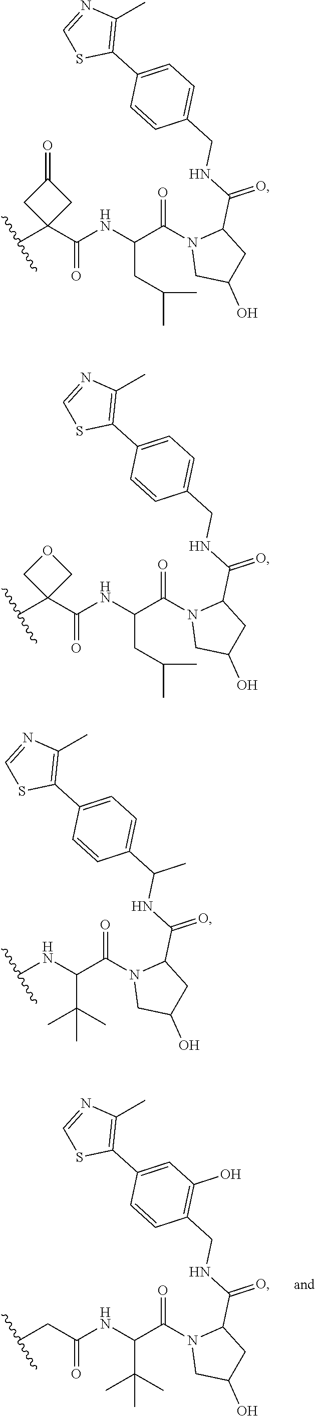 [SMILES: *C1(C(=O)NC(CC(C)C)C(=O)N2CC(O)CC2C(=O)NCc2ccc(-c3scnc3C)cc2)CC(=O)C1 ; *C1(C(=O)NC(CC(C)C)C(=O)N2CC(O)CC2C(=O)NCc2ccc(-c3scnc3C)cc2)COC1 ; *NC(C(=O)N1CC(O)CC1C(=O)NC(C)c1ccc(-c2scnc2C)cc1)C(C)(C)C ; *CC(=O)NC(C(=O)N1CC(O)CC1C(=O)NCc1ccc(-c2scnc2C)cc1O)C(C)(C)C ; "and"]

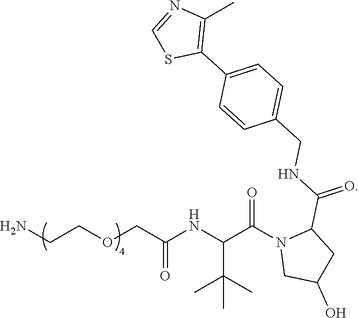

27. (canceled)

28. The compound of claim 1, wherein the compound has a structure represented by a formula: 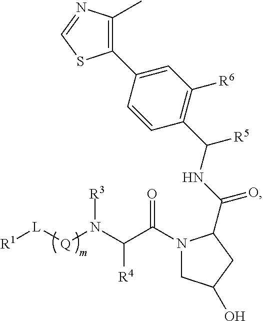
wherein m is 0 or 1;
wherein Q, when present, is —OC(O)—, —C(R^{10a})(R^{10b})C(O)—, —OC(R^{10a})(R^{10b})C(O)—, —C(R^{10a})(R^{10b})C(O)C(cyclopropyl)C(O)—, —C(R^{10a})(R^{10b})C(O)N(R^{11a})CH₂CH(R^{11b})C(O)—, —C(C3-C4 cycloalkyl)C(O)—, —NH(CH₂CH₂O)_{q}CH₂C(O)—, —NHCH₂C(cyclopropyl)C(O)—, or —CH₂C(O)N(R¹²)CH(R¹³)C(O)—;
wherein q, when present, is 1, 2, 3, 4, 5, or 6;
wherein each of R^{10a} and R^{10b}, when present, is independently hydrogen or C1-C4 alkyl;
or wherein each of R^{10a} and R^{10b}, when present, are covalently bound, and, together comprise a C3-C4 cycloalkyl or a C2-C3 heterocycloalkyl;
or wherein R¹⁰, when present, is covalently bound to R³, and, together with the intermediate atoms, comprises a 5-membered heterocycle;
wherein each of R^{11a} and R^{11b}, when present, are covalently bound, and, together with the intermediate atoms, comprise a 4-membered heterocycle;
wherein R¹², when present, is hydrogen; and
wherein R¹³, when present, is C1-C4 alkyl, —CH₂C₆H₅, or —C₆H₅;
or wherein each of R¹² and R¹³, when present, are covalently bound, and, together with the intermediate atoms, comprise an 10-membered heterocycloalkyl;
wherein R¹ is a 4,4-difluoro-4-bora-3a,4a-diaza-s-indacene (BODIPY) fluorophore;
wherein R³ is hydrogen or C1-C4 alkyl; and
wherein R⁴ is a C1-C4 alkyl, C1-C4 hydroxyalkyl, or C₆H₅;
or wherein each of R³ and R⁴ are covalently bound, and, together with the intermediate atoms, comprise a 5- or 6-membered heterocycle having 0 or 1 —OH group;
or wherein each of R³ and R¹⁰, when present, are covalently bound, and, together with the intermediate atoms, comprise a 5-membered heterocycle;
wherein R⁵ is hydrogen or methyl; and
wherein R⁶ is hydrogen, —OH, or C1-C4 alkyl halide.

29. (canceled)

30. The compound of claim 28, wherein the compound has a structure represented by a formula:

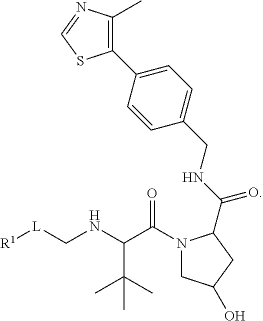

31. (canceled)

32. The compound of claim 28, wherein the compound has a structure represented by a formula:

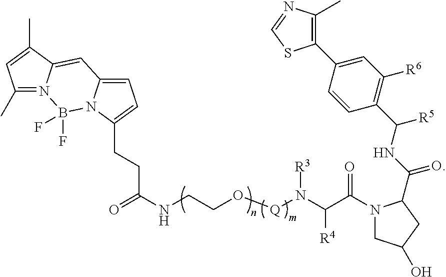

33. The compound of claim 28, wherein the compound has a structure represented by a formula:

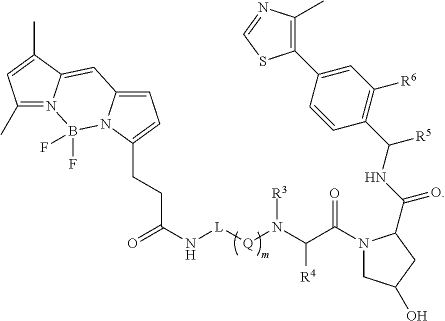

34. (canceled)

35. The compound of claim 1, wherein the compound is:

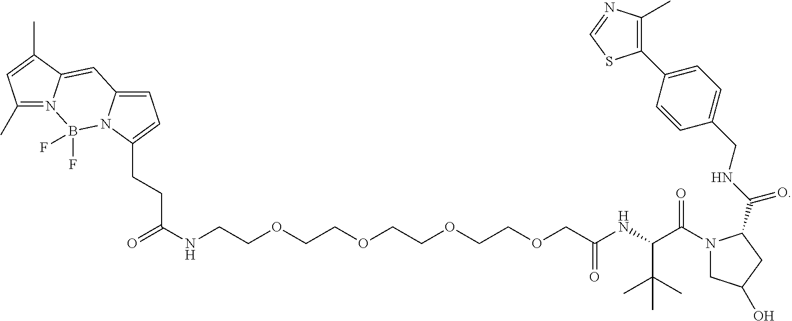

36. A method of modulating von Hippel-Lindau protein (pVHL) in a sample, the method comprising contacting the sample with an effective amount of the compound of claim 1, thereby modulating VHL protein in the sample.

37-52. (canceled)

53. A method of identifying a von Hippel-Lindau protein (pVHL) ligand in a library, the method comprising:
(a) providing a library that contains a plurality of ligands;
(b) combining the compound of claim 1 and a sample having pVHL, thereby forming a mixture;
(c) exposing each ligand to the mixture; and
(d) detecting a fluorescence emission of the mixture after exposure to each ligand,
wherein a decrease in fluorescence emission indicates that the ligand is a pVHL ligand, and
wherein a lack of decrease in fluorescence emission indicates that the ligand is a non-pVHL ligand.

54-55. (canceled)
